(19) United States
(12) Patent Application Publication
(10) Pub. No.: US 2022/0290179 A1
(43) Pub. Date: Sep. 15, 2022

(54) GENETICALLY ENGINEERED ONCOLYTIC VACCINIA VIRUSES AND METHODS OF USES THEREOF

(71) Applicant: Astellas Pharma Inc., Tokyo (JP)

(72) Inventors: Shinsuke Nakao, Tokyo (JP); Nobuaki Amino, Tokyo (JP); Yukinori Arai, Tokyo (JP)

(21) Appl. No.: 17/632,686

(22) PCT Filed: Aug. 27, 2020
(86) PCT No.: PCT/JP2020/034615

§ 371 Date: Feb. 3, 2022

Related U.S. Application Data
(60) Provisional application No. 62/893,316, filed on Aug. 29, 2019.

(51) Int. Cl. C12N 15/86; C12N 7/00; A61K 38/20; A61K 47/18; A61K 47/26; A61P 35/00; A61K 45/06; A61K 39/395; A61P 35/04

(52) U.S. Cl. CPC C12N 15/86; C12N 7/00; A61K 38/2046; A61K 38/208; A61K 47/18; A61K 47/26; A61P 35/00; A61K 45/06; A61K 39/39558; A61P 35/04; C12N 2710/24121; C12N 2710/24132; C12N 2710/24143; C12N 2710/24162; C12N 2710/24171

(57) ABSTRACT

The present invention provides pharmaceutical compositions comprising an oncolytic vaccinia virus and methods of using such pharmaceutical compositions for treating a subject having a cancer.

### RELATED APPLICATIONS

Ths application claims the benefit or priority to U.S. Provisional Application No. 62/893,316, filed on Aug. 29, 2019, the entire contents of which are incorporated herein by reference.

### TECHNICAL FIELD

This application is related to U.S. Patent Publication No. 2017/0340687, Japanese Patent Application Nos. JP 2018 223349 and JP 2018 179632, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Aug. 25, 2020, is named 127206_03920_SL.txt and is 4,095 bytes in size.

### BACKGROUND ART

### Background of the Invention

Various techniques for using viruses for cancer treatments have been recently developed. One such virus is vaccinia virus which has been studied as a vector for delivering therapeutic genes to cancer cells as an oncolytic virus that proliferates in cancer cells and destroys the cancer cells, or as a cancer vaccine that expresses tumor antigens or immunomodulatory molecules (Expert Opinion on Biological Therapy, 2011, vol. 11, p. 595-608).

Several vaccinia viruses have been engineered for use as oncolytic viruses (PCT Publication Nos. WO 2015/150809; and WO 2015/076422). However, an oncolytic vaccinia virus that expresses an immune-stimulating molecule may rapidly be cleared by the strong immune responses stimulated by the molecule and, thus, fail to be therapeutically effective. It is also believed that a strong immune response could serve either as a foe or as an ally to the vaccinia virus-mediated cancer therapy (Molecular Therapy, 2005, vol. 11, No. 2, p. 180-195).

Accordingly, there is a need in the art for oncolytic vaccinia viruses comprising polynucleotides expressing proteins that stimulate an immune response but that are not rapidly cleared and yet are therapeutically effective, pharmaceutical compositions comprising such oncolytic vaccinia viruses, and methods of use of such pharmaceutical compositions, alone or in combination with another agent or therapy, to treat a subject having a cancer.

### SUMMARY OF INVENTION

### Summary of the Invention

The present invention is based, at least in part, on the development of pharmaceutical compositions comprising an investigational oncolytic vaccinia virus and the discovery that such compositions are cytotoxic against various types of human cancer cell lines in vitro. The present invention is also based, at least in part, on the discovery that such pharmaceutical compostions have antitumor activity in vivo, that administration of the pharmaceutical compositions to a subject using a specific dosing regimen is very efficacious (e.g., the discovery that administration on days 1 and 15 is more efficacious as compared to a single administration), that administration of the pharmaceutical compositions to a subject induces intratumoral secretion of murine IL-12, human IL-7 and murine interferon gamma (IFN-γ) proteins and increased tumor infiltration with CD8+ T cells and CD4+ T cells, and that administration of the pharmaceutical compositions of the invention in combination with a checkpoint inhibitor, i.e., an anti-PD-1 antibody or an anti-CTLA4 antibody, induced higher antitumor activity than any of the treatments alone. The present invention is further based, at least in part, on the discovery that mice that achieved complete tumor regression (CR) following administration of the pharmaceutical compositions of the invention rejected the same cancer cells when re-challenged about 90 days after the CR, demonstrating establishment of antitumor immune memory. In addition, the present invention is based, at least in part, on the discovery that administration of the pharmaceutical compositions of the invention had an abscopal effect in a bilateral tumor model.

Accordingly, in one aspect, the present invention provides a pharmaceutical composition comprising about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; and a pharmaceutically acceptable carrier.

In one embodiment, the pharmaceutically acceptable carrier comprises tromethamine and sucrose.

In one embodiment, the pharmaceutically acceptable carrier comprises tromethamine at a concentration of about 10 mmol/L to about 50 mmol/L.

In one embodiment, the pharmaceutically acceptable carrier comprises sucrose at a concentration of about 5% w/v to about 15% w/v.

In one embodiment, the pH of the composition is about 5.0 to about 8.5.

In another aspect, the present invention provides a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; tromethamine at a concentration of about 10 mmol/L to about 50 mmol/L; and sucrose at a concentration of about 5% w/v to about 15% w/v, wherein the pH of the composition is about 5.0 to about 8.5.

In one embodiment, the deletion in the SCR domains in the B5R membrane protein extracellular region comprises a deletion in SCR domains 1-4.

In one embodiment, the deletion in the SCR domains of the B5R region comprises amino acid residues 22-237 of the amino acid sequence set forth in GenBank Accession No. AAA48316.1.

In one embodiment, the gene encoding the SCR domain-deleted B5R region is a gene encoding a polypeptide containing the signal peptide, stalk, transmembrane, and cytoplasmic tail domains of the B5R region.

In one embodiment, the SCR domain-deleted B5R region comprises the amino acid sequence of the B5R region corresponding to the amino acid sequence set forth in SEQ ID NO: 2.

In one embodiment, the vaccinia virus is a LC16mo strain of virus.

In one embodiment, the oncolytic vaccinia virus is LC16mO ΔSCR VGF-SP-IL12/O1L-SP-IL7.

The pharmaceutical composition of the invention may comprise about 1×10⁷ to about 1×10⁹ particle forming units (pfu)/ml of the oncolytic vaccinia virus; about 1×10⁷ particle forming units (pfu)/ml of the oncolytic vaccinia virus; about 5×10⁷ particle forming units (pfu)/ml of the oncolytic vaccinia virus; about 1×10⁸ particle forming units (pfu)/ml of the oncolytic vaccinia virus; about 5×10⁸ particle forming units (pfu)/ml of the oncolytic vaccinia virus; about 1×10⁹ particle forming units (pfu)/ml of the oncolytic vaccinia virus; or about 5×10⁹ particle forming units (pfu)/ml of the oncolytic vaccinia virus.

The pharmaceutical composition of the invention may comprise tromethamine at a concentration of about 15 mmol/L to about 45 mmol/L; 20 mmol/L to about 40 mmol/L; or 25 mmol/L to about 35 mmol/L. In one embodiment, the concentration of tromethamine is about 30 mmol/L.

The pharmaceutical composition of the invention may comprise sucrose at a concentration of about 6% w/v to about 14% w/v; about 7% w/v to about 13% w/v; about 8% w/v to about 12% w/v; or about 9% w/v to about 11% w/v. In one embodiment, the concentration of sucrose is about 10% w/v.

The pH of the pharmaceutical composition may be about 8.0; about 6.5 to about 8.0; or about 6.8 to about 7.8. In one embodiment, the pH of the composition is about 7.6.

In one embodiment, the composition is stable for at least about 6 months to about 2 years when stored at about −70° C.

The present invention also provides a vial and a syringe comprising any of the pharmaceutical compositions of the invention.

In one aspect, the present invention provides a method of treating a subject having a cancer. The method includes administering to the subject a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; and a pharmaceutically acceptable carrier, thereby treating the subject.

In another aspect, the present invention provides a method of treating a subject having a cancer. The method includes administering to the subject a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; and a pharmaceutically acceptable carrier, wherein administration of the pharmaceutical composition to the subject induces an abscopal effect, thereby treating the subject.

In another aspect, the present invention provides a method of inducing an abscopal effect in a subject having a cancer. The method includes administering to the subject a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; and a pharmaceutically acceptable carrier, thereby inducing an abscopal effect in a subject having a cancer.

In one aspect, the present invention method of treating a subject having a cancer. The method includes administering to the subject a therapeutically effective amount of a pharmaceutical composition, comprising about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; tromethamine at a concentration of about 10 mmol/L to about 50 mmol/L; and sucrose at a concentration of about 5% w/v to about 15% w/v, wherein the pH of the composition is about 5.0 to about 8.5, thereby treating the subject.

In another aspect, the present invention provides a method of treating a subject having a cancer. The method includes administering to the subject a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; tromethamine at a concentration of about 10 mmol/L to about 50 mmol/L; and sucrose at a concentration of about 5% w/v to about 15% w/v, wherein the pH of the composition is about 5.0 to about 8.5, and wherein administration of the pharmaceutical composition to the subject induces an abscopal effect, thereby treating the subject.

In another aspect, the present invention provides a method of inducing an abscopal effect in a subject having a cancer. The method includes administering to the subject a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., LC16mO ΔSCR VGF-SP-IL12/O1L-SP-IL7; tromethamine at a concentration of about 10 mmol/L to about 50 mmol/L; and sucrose at a concentration of about 5% w/v to about 15% w/v, wherein the pH of the composition is about 5.0 to about 8.5, and wherein administration of the pharmaceutical composition to the subject induces an abscopal effect, thereby inducing an abscopal effect in the subject.

In one embodiment, the abscopal effect occurs in a metastatic tumor that is proximate to a primary solid tumor.

In another embodiment, the abscopal effect occurs in a metastatic tumor that is remote to a primary solid tumor.

In one embodiment, the oncolytic vaccinia virus is LC16mO ΔSCR VGF-SP-IL12/O1L-SP-IL7.

The subject may be administered a dose of about 1×10⁷ to about 1×10⁹ particle forming units (pfu); a dose of about 1×10⁷ particle forming units (pfu); a dose of about 5×10⁷ particle forming units (pfu); a dose of about 1×10⁸ particle forming units (pfu); a dose of about 5×10⁸ particle forming units (pfu); or a dose of about 1×10⁹ particle forming units (pfu).

In one embodiment, the administration is intratumoral administration.

In one embodiment, the dose of the pharmaceutical composition is administered to the subject intratumorally in a volume that achieves an injection ratio of about 0.2 to about 0.8 (volume of pharmaceutical composition/tumor volume).

The pharmaceutical composition may be administered to the subject once about once every week, once every two weeks, once every three weeks, or once every four weeks. In one embodiment, the pharmaceutical composition is administered to the subject once about once every two weeks.

The pharmaceutical composition may be administered to the subject in a dosing regimen.

In one embodiment, the dosing regimen comprises administering to the subject a first dose of the pharmaceutical composition on day 1 and a second dose of the pharmaceutical composition on day 15.

In one embodiment, the dosing regimen is repeated beginning at day 28 following the first dose of the pharmaceutical composition.

In one embodiment, the cancer is a primary tumor, such as a solid tumor. In one embodiment, the solid tumor is an advanced solid tumor.

In one embodiment, the cancer is a metastatic tumor.

In one embodiment, the cancer is a cutaneous, subcutaneous, mucosal or submucosal tumor.

In one embodiment, the cancer is a primary or metastatic solid tumor in a location other than a cutaneous, a subcutaneous, a mucosal or a submucosal location.

In one embodiment, the cancer is a head and neck squamous cell carcinoma, a dermatological cancer, a nasopharyngeal cancer, a sarcoma, or a genitourinary/gynecological tumor.

In one embodiment, the cancer is a primary or metastatic tumor of the liver.

In one embodiment, the cancer is a primary or metastatic gastric tumor.

In one embodiment, the cancer the cancer is malignant melanoma, lung adenocarcinoma, lung cancer, small cell lung cancer, lung squamous carcinoma, kidney cancer, bladder cancer, head and neck cancer, breast cancer, esophageal cancer, glioblastoma, neuroblastoma, myeloma, ovarian cancer, colorectal cancer, pancreatic cancer, prostate cancer, hepatocellular carcinoma, mesothelioma, cervical cancer or gastric cancer.

In one embodiment, the subject is human.

The human subject may be an adult subject; an adolescent subject; or a pediatric subject.

In one embodiment, administration of the pharmaceutical composition to the subject leads to at least one effect selected from the group consisting of inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, antitumor immunity, and stable disease.

The methods of the invention may further comprise administering to the subject an additional therapeutic agent or therapy,

In one embodiment, the additional therapeutic agent or therapy, is selected from the group consisting of surgery, radiation, a chemotherapeutic agent, a cancer vaccine, a checkpoint inhibitor, a lymphocyte activation gene 3 (LAG3) inhibitor, a glucocorticoid-induced tumor necrosis factor receptor (GITR) inhibitor, a T-cell immunoglobulin and mucin-domain containing-3 (TIM3) inhibitor, a B- and T-lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a bispecific anti-CD3/anti-CD20 antibody, a vascular endothelial growth factor (VEGF) antagonist, an angiopoietin-2 (Ang2) inhibitor, a transforming growth factor beta (TGFβ) inhibitor, a CD38 inhibitor, an epidermal growth factor receptor (EGFR) inhibitor, granulocyte-macrophage colony stimulating factor (GM-CSF), cyclophosphamide, an antibody to a tumor-specific antigen, Bacillus Calmette-Guerin vaccine, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement.

The methods of the invention may further comprise administering to the subject a therapeutically effective amount of a checkpoint inhibitor.

In one embodiment, the checkpoint inhibitor is a programmed cell death 1 (PD-1) inhibitor; a programmed cell death ligand 1 (PD-L1) inhibitor; a cytotoxic T lymphocyte associated protein 4 (CTLA-4) inhibitor; a T-cell immunoglobulin domain and mucin domain-3 (TIM-3) inhibitor; a lymphocyte activation gene 3 (LAG-3) inhibitor; a T cell immunoreceptor with Ig and ITIM domains (TIGIT) inhibitor; a B and T lymphocyte associated (BTLA) inhibitor; or a V-type immunoglobulin domain-containing suppressor of T-cell activation (VISTA) inhibitor.

In one embodiment, the checkpoint inhibitor is a programmed cell death 1 (PD-1) inhibitor, a programmed cell death ligand 1 (PD-L1) inhibitor, or a cytotoxic T lymphocyte associated protein 4 (CTLA-4) inhibitor.

In one embodiment, the checkpoint inhibitor is selected from the group consisting of an anti-PD-1 antibody, or antigen-binding fragment thereof; an anti-PD-L1 antibody, or antigen-binding fragment thereof; an anti-CTLA-4 antibody, or antigen-binding fragment thereof; an anti-TIM-3 antibody, or antigen-binding fragment thereof; an anti-LAG-3 antibody, or antigen-binding fragment thereof; an anti-TIGIT antibody, or antigen-binding fragment thereof; an anti-BTLA antibody, or antigen-binding fragment thereof; and an anti-VISTA antibody, or antigen-binding fragment thereof.

In one embodiment, the checkpoint inhibitor is an anti-programmed cell death 1 (PD-1) antibody, or antigen-binding fragment thereof; an anti-programmed cell death ligand 1 (PD-L1) antibody, or antigen-binding fragment thereof; or an anti-cytotoxic T lymphocyte associated protein 4 (CTLA-4) antibody, or antigen-binding fragment thereof.

In one embodiment, the anti-PD-1 antibody is nivolumab or pembrolizumab.

In one embodiment, the anti-PD-L1 antibody is atezolizumab.

In one embodiment, the anti-CTLA-4 antibody is ipilimumab.

In one aspect, the present invention provides a pharmaceutical composition comprising, about 1×10⁶ to about 1×10¹⁰ particle forming units (pfu)/ml of LC16mO ΔSCR VGF-SP-IL12/O1L-SP-IL7; tromethamine at a concentration of about 30 mmol/L; and sucrose at a concentration of about 10% w/v, wherein the pH of the composition is about 7.6.

In one aspect, the present invention provides a method of treating a subject having a cancer. The method includes administering to the subject a therapeutically effective amount of a pharmaceutical composition comprising, about 1×10⁶ to about 1×10¹⁰ particle forming units (pfu)/ml of LC16mO ΔSCR VGF-SP-IL12/O1L-SP-IL7; tromethamine at a concentration of about 30 mmol/L; and sucrose at a concentration of about 10% w/v, wherein the pH of the composition is about 7.6, thereby treating the subject.

In another aspect, the present invention provides a method of treating a subject having a cancer. The method includes administering to the subject a therapeutically effective amount of a pharmaceutical composition comprising, about 1×10⁶ to about 1×10¹⁰ particle forming units (pfu)/ml of LC16mO ΔSCR VGF-SP-IL12/O1L-SP-IL7; tromethamine at a concentration of about 30 mmol/L; and sucrose at a concentration of about 10% w/v, wherein the pH of the composition is about 7.6, and wherein administration of the pharmaceutical composition to the subject induces an abscopal effect, thereby treating the subject.

In one aspect, the present invention provides a method of inducing an abscopal effect in a subject having a cancer. The method includes administering to the subject a therapeutically effective amount of a pharmaceutical composition comprising, about 1×10⁶ to about 1×10¹⁰ particle forming units (pfu)/ml of LC16mO ΔSCR VGF-SP-IL12/O1L-SP-IL7; tromethamine at a concentration of about 30 mmol/L; and sucrose at a concentration of about 10% w/v, wherein the pH of the composition is about 7.6, and wherein administration of the pharmaceutical composition to the subject induces an abscopal effect, thereby inducing an abscopal effect in the subject.

### BRIEF DESCRIPTION OF DRAWINGS

### Brief Description of the Drawings

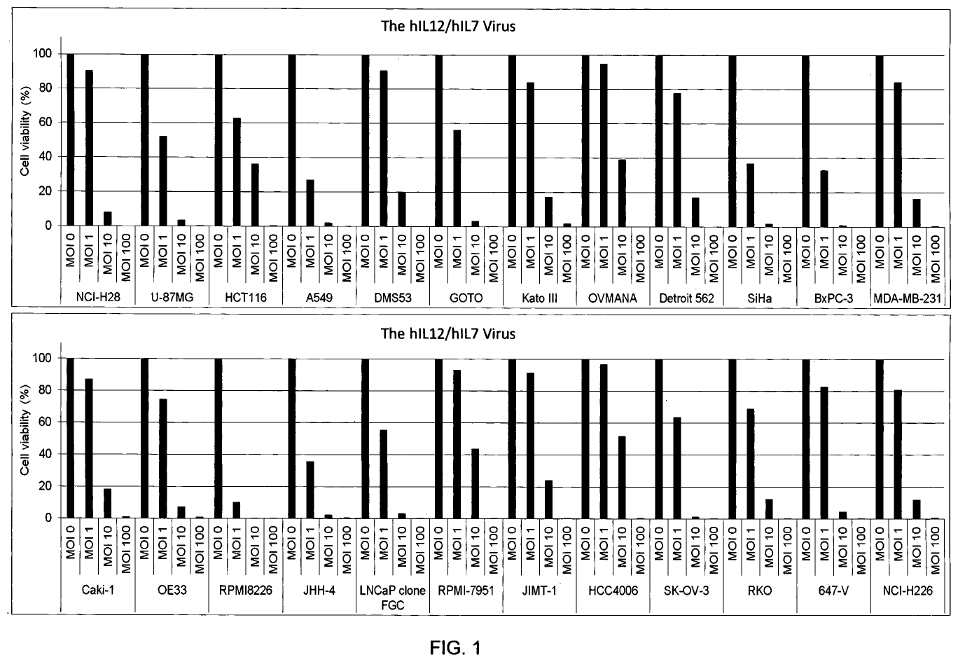

FIG. 1 depicts a series of graphs depicting the cytotoxic effect of the hIL12 and hIL7-carrying vaccinia virus against human cancer cell lines. The human cell lines used were: Human cancer cell lines: NCI-H28 (mesothelioma), U-87 MG (glioblastoma), HCT 116 (colorectal carcinoma), A549 (lung carcinoma), DMS 53 (small cell lung cancer cell), GOTO (neuroblastoma), Kato III (gastric cancer cell), OVMANA (ovarian cancer cell), Detroit 562 (head and neck cancer cell), SiHa (cervical cancer cell), BxPC-3 (pancreatic cancer cell), MDA-MB-231 (breast cancer cell), Caki-1 (kidney cancer cell), OE33 (esophageal cancer cell), RPMI 8226 (myeloma), JHH-4 (hepatocellular carcinoma), LNCaP clone FGC (prostate cancer cell), RPMI-7951 (malignant melanoma), JIMT-1 (breast cancer cell), HCC4006 (lung adenocarcinoma), SK-OV-3 (ovarian cancer cell), RKO (colon cancer cell), 647-V (bladder cancer cell) and NCI-H226 (lung squamous cell carcinoma).

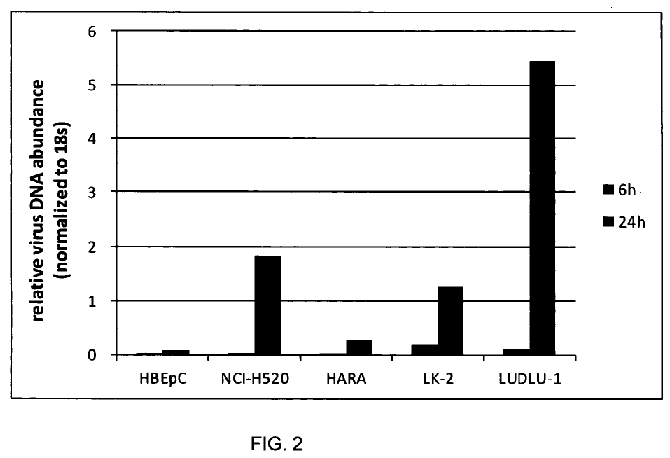

FIG. 2 is a graph depicting the replication of the hIL12 and hIL7-carrying vaccinia virus genome in human cancer cells or normal cells. Values were normalized to the 18s ribosomal RNA gene and expressed as the mean of duplicate measures. NCI-H520, HARA, LK-2 and LUDLU-1 are human cancer cell lines.

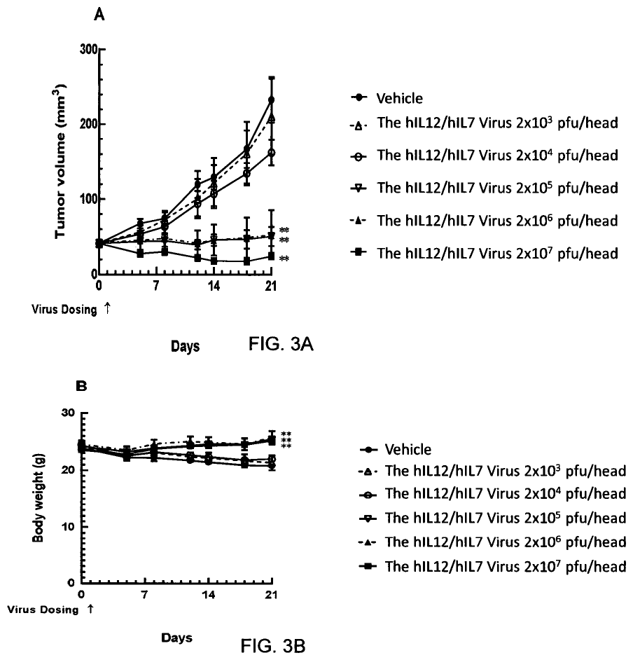

FIG. 3A is a graph depicting tumor growth change (tumor volume) in COLO 741 Tumor cell-bearing mice treated with the hIL12 and hIL7-carrying vaccinia virus. Each point represents the mean±SEM (n=6). Statistical analysis was performed for the values on day 21. COLO 741: human colorectal carcinoma cell line; Vehicle: 30 mmol/L Tris-HCl containing 10% sucrose. **P<0.01 compared with the vehicle treatment group (Dunnett's multiple comparison test).

FIG. 3B is a graph depicting body weight change in COLO 741 Tumor cell-bearing mice treated with the hIL12 and hIL7-carrying vaccinia virus. Each point represents the mean z SEM (n=6). **P<0.01 compared with the vehicle treatment group (Dunnett's multiple comparison test).

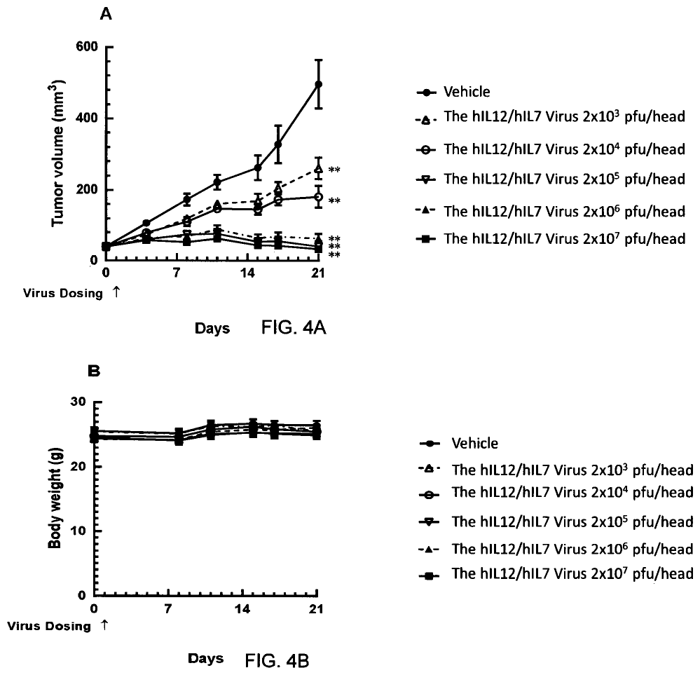

FIG. 4A is a graph depicting tumor growth (tumore volume) a change in U-87 MG-bearing mice treated with the hIL12 and hIL7-carrying vaccinia virus. Each point represents the mean±SEM (n=6). Statistical analysis was performed for the values on day 21. U-87 MG: human glioblastoma cell line; Vehicle: 30 mmol/L Tris-HCl containing 10% sucrose. **P<0.01 compared with the vehicle treatment group (Dunnett's multiple comparison test).

FIG. 4B is a graph depicting body weight change in U-87 MG-bearing mice treated with the hIL12 and hIL7-carrying vaccinia virus. Each point represents the mean±SEM (n=6). There was no significant body weight loss between the vehicle treatment group and the the hIL12 and hIL7-carrying vaccinia virus treatment groups on day 21 (Dunnett's multiple comparison test). U-87 MG: human glioblastoma cell line; Vehicle: 30 mmol/L Tris-HCl containing 10% sucrose.

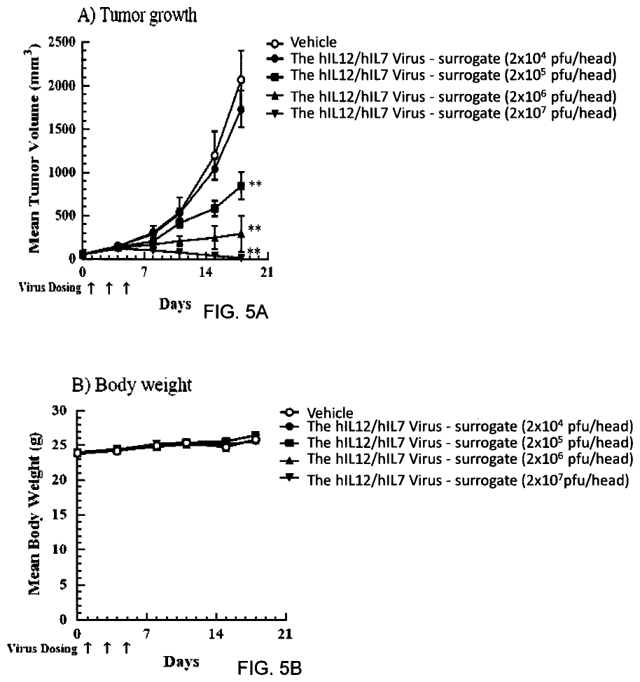

FIG. 5A is a graph depicting tumor growth change (tumor volume) in CT26.WT tumor cell-bearing mice treated with the hIL12 and hIL7-carrying vaccinia virus-surrogate. Each value represents the mean±SEM (n=6). Vehicle or the hIL12 and hIL7-carrying vaccinia virus-surrogate at the indicated doses was intratumorally injected on days 1, 3 and 5 in mice inoculated with CT26.WT tumor cells. Statistical analysis was performed using the values of tumor volume on day 18. CT26.WT: murine colorectal carcinoma cell line; Vehicle: 30 mmol/L Tris-HCl containing 10% sucrose. **P<0.01 versus the vehicle control group (Dunnett's multiple comparison test).

FIG. 5B is a graph depicting body weight changes in CT26.WT tumor cell-bearing mice treated with the hIL12 and hIL7-carrying vaccinia virus-surrogate. Each value represents the mean±SEM (n=6). Vehicle or the hIL12 and hIL7-carrying vaccinia virus-surrogate at the indicated doses was intratumorally injected on days 1, 3 and 5 in mice inoculated with CT26.WT tumor cells. Statistical analysis was performed using the values of tumor volume on day 18. CT26.WT: murine colorectal carcinoma cell line; Vehicle: 30 mmol/L Tris-HCl containing 10% sucrose. **P<0.01 versus the vehicle control group (Dunnett's multiple comparison test).

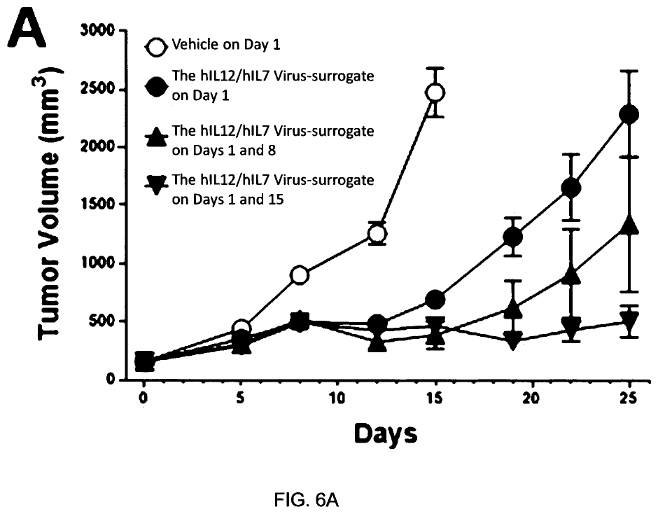

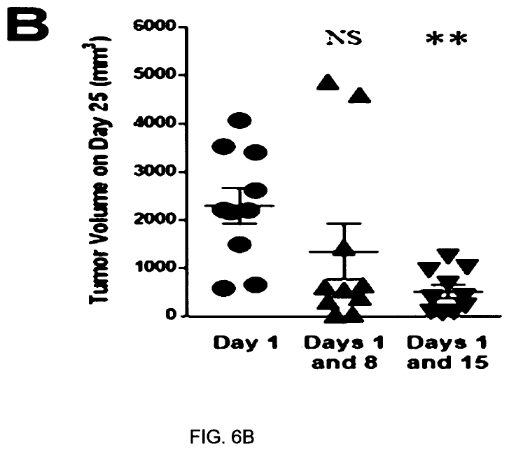

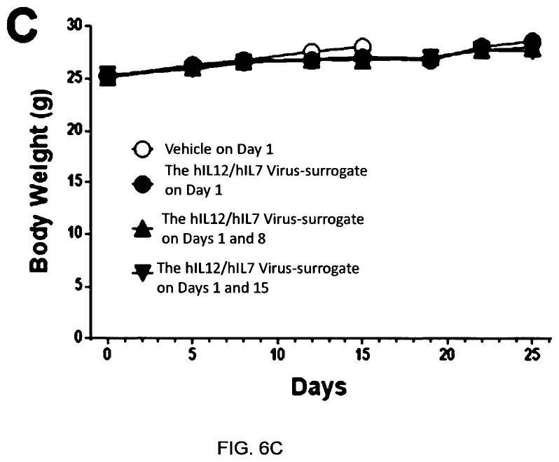

FIGS. 6A-6C are graphs depicting the effects of intratumoral administration of the hIL12 and hIL7-carrying vaccinia virus-surrogate on (6A) Tumor growth (tumor volume), (6B) Tumor growth (tumor volume) on day 25, and (6C) Body weight.

FIG. 6A is a graph depicting the antitumor effects of intratumoral administration of the hIL12 and hIL7-carrying vaccinia virus-surrogate on day 1 in immunocompetent mice with CT26.WT tumors. Each point represents the mean±SEM (n=10). CT26.WT: murine colorectal carcinoma cell line. **P<0.01, NS: not significant versus the hIL12 and hIL7-carrying vaccinia virus-surrogate single-dose group (Dunnett's multiple comparison test) on day 25.

FIG. 6B is a graph depicting antitumor effects of intratumoral administration of the hIL12 and hIL7-carrying vaccinia virus-surrogate on days 1 and 8 in immunocompetent mice with CT26.WT tumors. Each point represents the mean±SEM (n=10). CT26.WT: murine colorectal carcinoma cell line. **P<0.01, NS: not significant versus the hIL12 and hIL7-carrying vaccinia virus-surrogate single-dose group (Dunnett's multiple comparison test) on day 25.

FIG. 6C is a graph depicting antitumor effects of intratumoral administration of the hIL12 and hIL7-carrying vaccinia virus-surrogate on days 1 and 15 in immunocompetent mice with CT26.WT tumors. Each point represents the mean±SEM (n=10). CT26.WT: murine colorectal carcinoma cell line. **P<0.01, NS: not significant versus the hIL12 and hIL7-carrying vaccinia virus-surrogate single-dose group (Dunnett's multiple comparison test) on day 25.

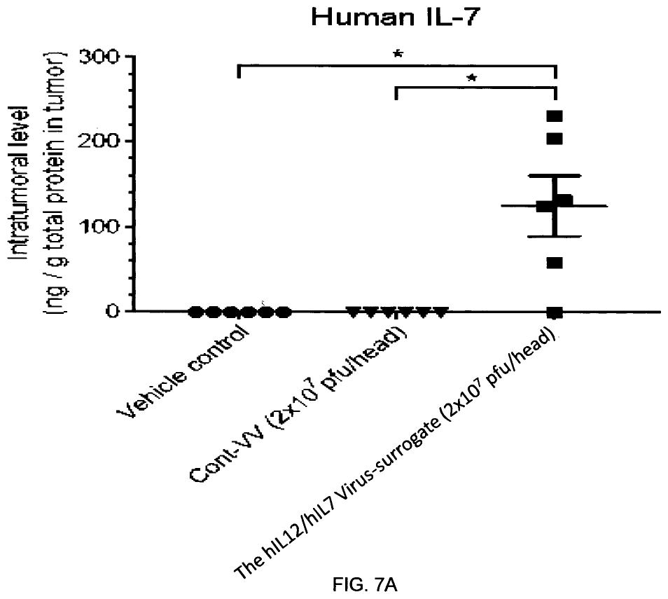

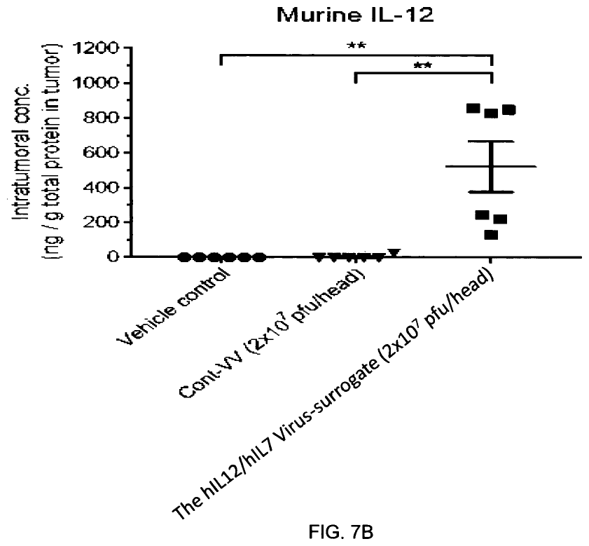

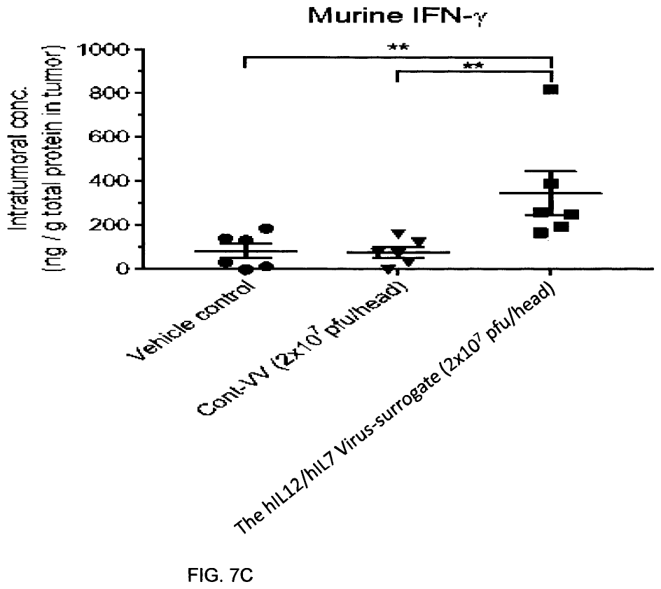

FIG. 7A is a graph depicting levels of human IL-7 in tumors. Cont-VV or the hIL12 and hIL7-carrying vaccinia virus-surrogate. IL-7: interleukin-7; Cont-VV: recombinant vaccinia virus carrying no immune transgene; Vehicle: 30 mmol/L Tris-HCl containing 10% sucrose.*, **P<0.05, 0.01 (Mann-Whitney U-test).

FIG. 7B is a graph depicting levels of murine IL-12 in tumors. Cont-VV or the hIL12 and hIL7-carrying vaccinia virus-surrogate. IL-12: interleukin-12; Cont-VV: recombinant vaccinia virus carrying no immune transgene; Vehicle: 30 mmol/L Tris-HCl containing 10% sucrose.*, **P<0.05, 0.01 (Mann-Whitney U-test).

FIG. 7C is a graph depicting levels of murine IFN-γ in tumors. Cont-VV or the hIL12 and hIL7-carrying vaccinia virus-surrogate. IFN-γ: interferon gamma; Cont-VV: recombinant vaccinia virus carrying no immune transgene; Vehicle: 30 mmol/L Tris-HCl containing 10% sucrose.*, **P<0.05, 0.01 (Mann-Whitney U-test).

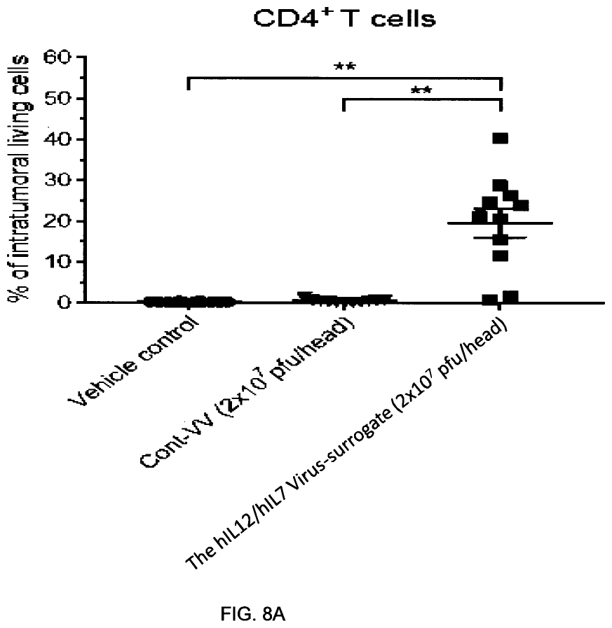

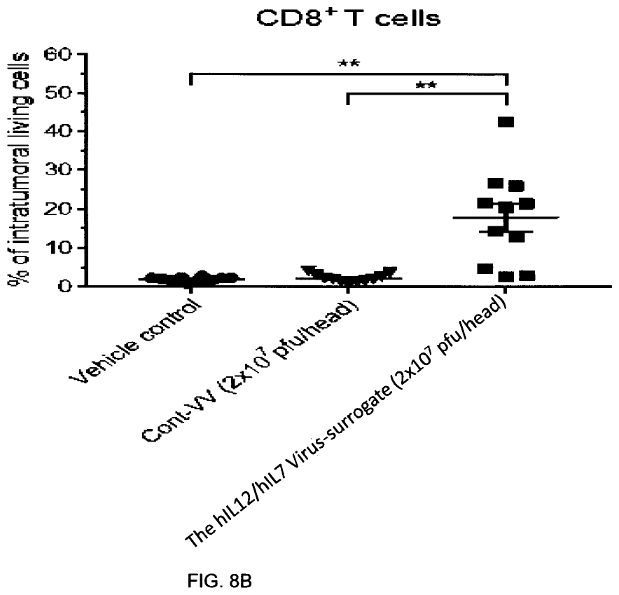

FIG. 8A is a graph depicting murine CD4+ T cells in tumor. Each point represents the mean±SEM (n=12 for vehicle, n=11 for Cont-VV and the hIL12 and hIL7-carrying vaccinia virus-surrogate). CD4: surface antigen specific for the T helper cell subpopulation; Cont-VV: recombinant vaccinia virus carrying no immune transgene; Vehicle: 30 mmol/L Tris-HCl containing 10% sucrose. **P<0.01 (Mann-Whitney U-test).

FIG. 8B is a graph depicting murine CD8+ T cells in tumor. Each point represents the mean±SEM (n=12 for vehicle, n=11 for Cont-VV and the hIL12 and hIL7-carrying vaccinia virus-surrogate). CD8: surface antigen presented on cytotoxic T cells; Cont-VV: recombinant vaccinia virus carrying no immune transgene; Vehicle: 30 mmol/L Tris-HCl containing 10% sucrose. **P<0.01 (Mann-Whitney U-test).

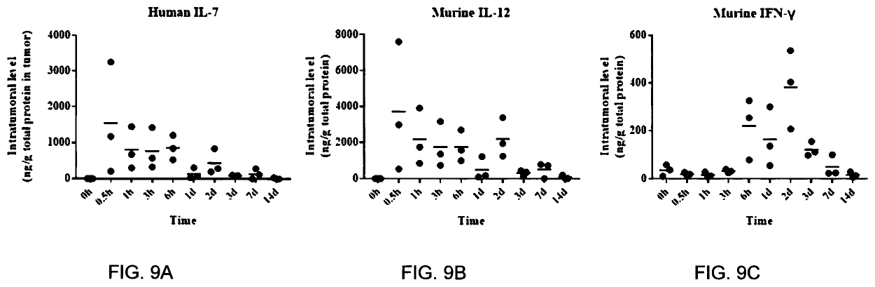

FIGS. 9A-9C are dot plot graphs depicting individual measurement values of human IL-7 (A), murine IL-12 (B) and murine IFN-γ (C) in tumor samples from CT26.WT tumor-bearing mice treated with the hIL12 and hIL7-carrying vaccinia virus-surrogate.

FIG. 9A is a graph depicting tumor levels of human IL-7 in CT26.WT tumor-bearing mice following intratumoral injection of the hIL12 and hIL7-carrying vaccinia virus-surrogate. Horizontal bar indicates the mean of 3 animals. CT26.WT: murine colorectal carcinoma cell line, the hIL12 and hIL7-carrying vaccinia virus-surrogate: recombinant vaccinia virus carrying murine IL-12 gene and human IL-7 gene. ELISA: enzyme-linked immunosorbent assay; IL-7: interleukin-7; MSD: Meso Scale Discovery.

FIG. 9B is a graph depicting tumor levels of murine IL-12 in CT26.WT tumor-bearing mice following intratumoral injection of the hIL12 and hIL7-carrying vaccinia virus-surrogate. Horizontal bar indicates the mean of 3 animals. CT26.WT: murine colorectal carcinoma cell line, the hIL12 and hIL7-carrying vaccinia virus-surrogate: recombinant vaccinia virus carrying murine IL-12 gene and human IL-7 gene. ELISA: enzyme-linked immunosorbent assay; IL-12: interleukin-12; MSD: Meso Scale Discovery.

FIG. 9C is a graph depicting tumor levels of murine IFN-γ in CT26.WT tumor-bearing mice following intratumoral injection of the hIL12 and hIL7-carrying vaccinia virus-surrogate. Horizontal bar indicates the mean of 3 animals. CT26.WT: murine colorectal carcinoma cell line, the hIL12 and hIL7-carrying vaccinia virus-surrogate: recombinant vaccinia virus carrying murine IL-12 gene and human IL-7 gene. ELISA: enzyme-linked immunosorbent assay; IFN-γ: interferon gamma; MSD: Meso Scale Discovery.

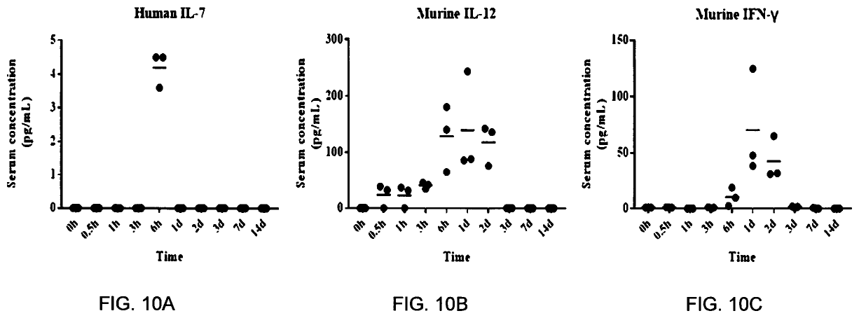

FIGS. 10A-10C are dot plot graphs depicting individual measurement values of human IL-7 (A), murine IL-12 (B) and murine IFN-γ (C) in serum samples from CT26.WT tumor-bearing mice treated with the hIL12 and hIL7-carrying vaccinia virus-surrogate.

FIG. 10A is a graph depicting serum levels of human IL-7 in CT26.WT tumor-bearing mice following intratumoral injection of the hIL12 and hIL7-carrying vaccinia virus-surrogate. Horizontal bar indicates the mean of 3 animals. CT26.WT: murine colorectal carcinoma cell line, the hIL12 and hIL7-carrying vaccinia virus-surrogate: recombinant vaccinia virus carrying murine IL-12 gene and human IL-7 gene. ELISA: enzyme-linked immunosorbent assay; IL-7: interleukin-7; MSD: Meso Scale Discovery.

FIG. 10B is a graph depicting serum levels of murine IL-12 in CT26.WT tumor-bearing mice following intratumoral injection of the hIL12 and hIL7-carrying vaccinia virus-surrogate. Horizontal bar indicates the mean of 3 animals. CT26.WT: murine colorectal carcinoma cell line, the hIL12 and hIL7-carrying vaccinia virus-surrogate: recombinant vaccinia virus carrying murine IL-12 gene and human IL-7 gene. ELISA: enzyme-linked immunosorbent assay; IL-12: interleukin-12; MSD: Meso Scale Discovery.

FIG. 10C is a graph depicting serum levels of murine IFN-γ in CT26.WT tumor-bearing mice following intratumoral injection of the hIL12 and hIL7-carrying vaccinia virus-surrogate. Horizontal bar indicates the mean of 3 animals. CT26.WT: murine colorectal carcinoma cell line, the hIL12 and hIL7-carrying vaccinia virus-surrogate: recombinant vaccinia virus carrying murine IL-12 gene and human IL-7 gene. ELISA: enzyme-linked immunosorbent assay; IFN-γ: interferon gamma; MSD: Meso Scale Discovery.

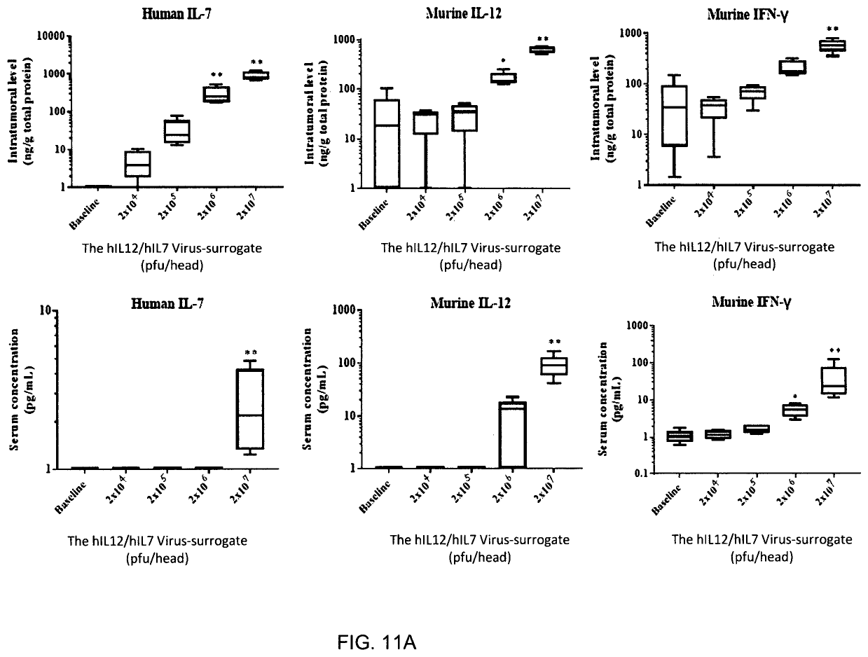

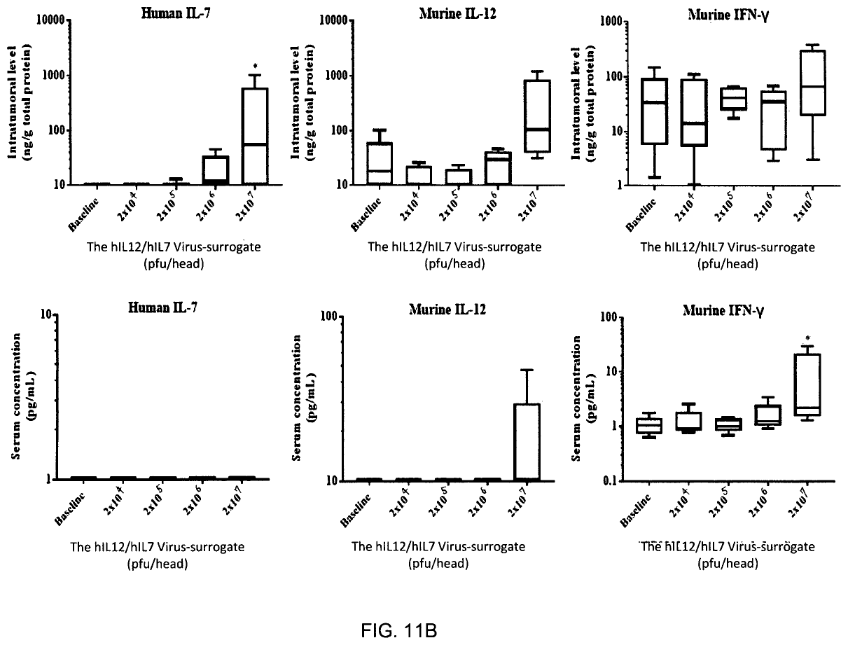

FIG. 11A are graphs depicting tumor and serum human IL-7, murine IL-12 and murine IFN-γ levels after the hIL12 and hIL7-carrying vaccinia virus-surrogate single intratumoral injection. Box plots represent the median, interquartile range, maximum and minimum. The hIL12 and hIL7-carrying vaccinia virus-surrogate: recombinant vaccinia virus carrying murine IL-12 and human IL-7 genes; CT26.WT: murine colorectal carcinoma cell line; IFN-γ: interferon gamma; IL-7: interleukin-7; IL-12: interleukin-12; MSD: Meso Scale Discovery.

FIG. 11B are graphs depicting tumor and serum human IL-7, murine IL-12 and murine IFN-γ levels after the hIL12 and hIL7-carrying vaccinia virus-surrogate single intratumoral injection. Box plots represent the median, interquartile range, maximum and minimum. The hIL12 and hIL7-carrying vaccinia virus-surrogate: recombinant vaccinia virus carrying murine IL-12 and human IL-7 genes; CT26.WT: murine colorectal carcinoma cell line; IFN-γ: interferon gamma; IL-7: interleukin-7; IL-12: interleukin-12; MSD: Meso Scale Discovery.

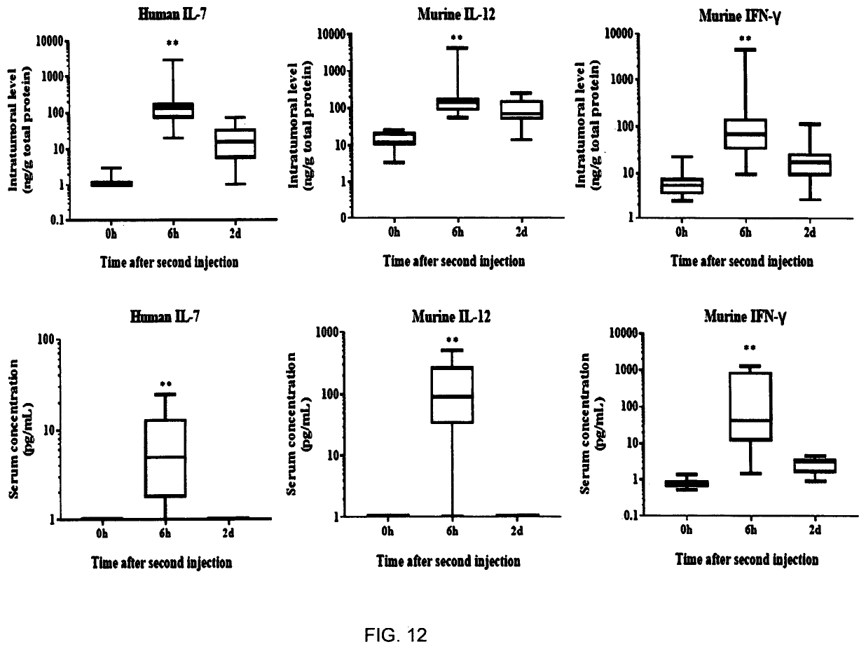

FIG. 12 are graphs depicting tumor and serum human IL-7, murine IL-12 and murine IFN-γ levels after the hIL12 and hIL7-carrying vaccinia virus-surrogate repeated intratumoral injections. Box plots represent the median, inter-quartile range, maximum and minimum. Significance was determined at **P<0.01. The hIL12 and hIL7-carrying vaccinia virus-surrogate: recombinant vaccinia virus carrying murine IL-12 and human IL-7 genes; CT26.WT: murine colorectal carcinoma cell line; IFN-γ: interferon gamma; IL-7: interleukin-7; IL-12: interleukin-12; MSD: Meso Scale Discovery.

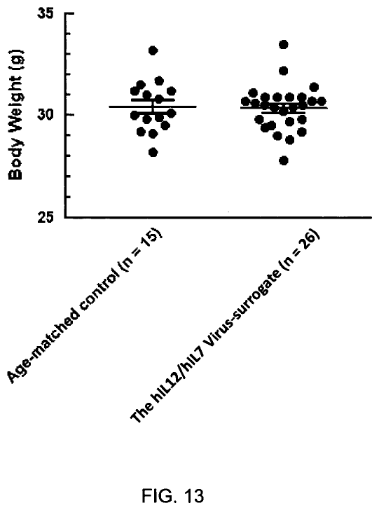

FIG. 13 is a graph depicting a comparison in body weight of mice had achieved CR at 90 Days after completion of the hIL12 and hIL7-carrying vaccinia virus-surrogate injection and age-matched control mice. Dot plots represent individual body weight of mice that achieved CR at 90 days after the final injection of the hIL12 and hIL7-carrying vaccinia virus-surrogate and the age-matched control mice. Horizontal line and vertical bar in each group indicate the mean and SEM, respectively. There was no significant difference in body weight between the mice had induced CR and the age-matched control mice (unpaired Student's t-test). CR: complete tumor regression; CT26.WT: murine colorectal carcinoma cell line.

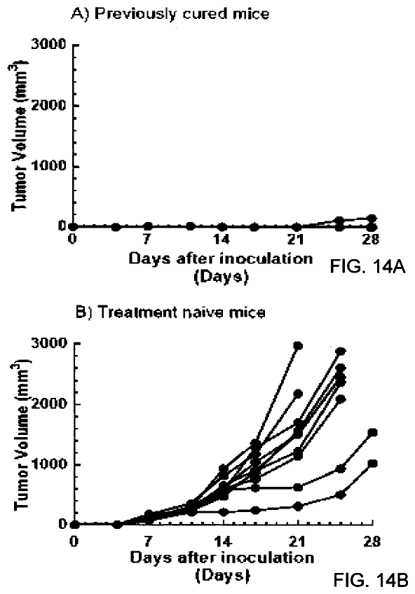

FIGS. 14A-14B are graphs depicting tumor growth (tumor volume) of individual mice after inoculation with CT26.WT tumor cells. Mice that achieved CR of CT26.WT tumor cells after the hIL12 and hIL7-carrying vaccinia virus-surrogate treatment (previously cured mice; FIG. 14A) and age-matched control mice (treatment-naive mice; FIG. 14B) were subcutaneously inoculated with CT26.WT tumor cells at 5×10⁵ cells/mouse (n=10) and were observed for 28 days after the inoculation. CR: complete tumor regression; CT26.WT: murine colorectal carcinoma cell line.

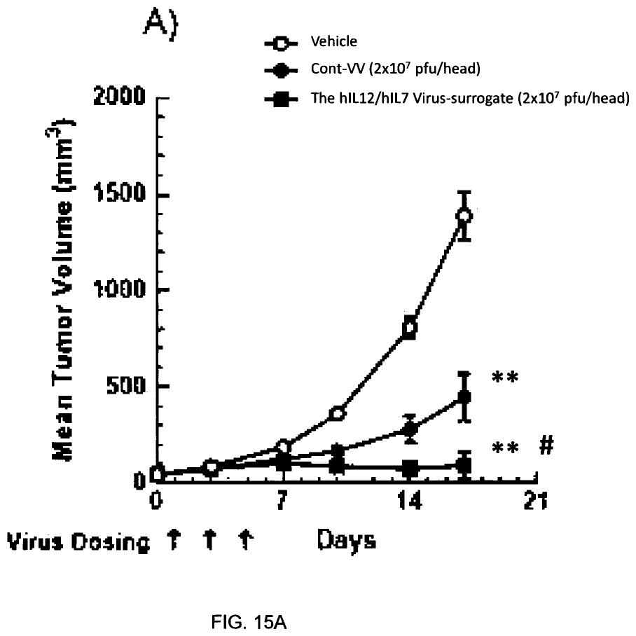

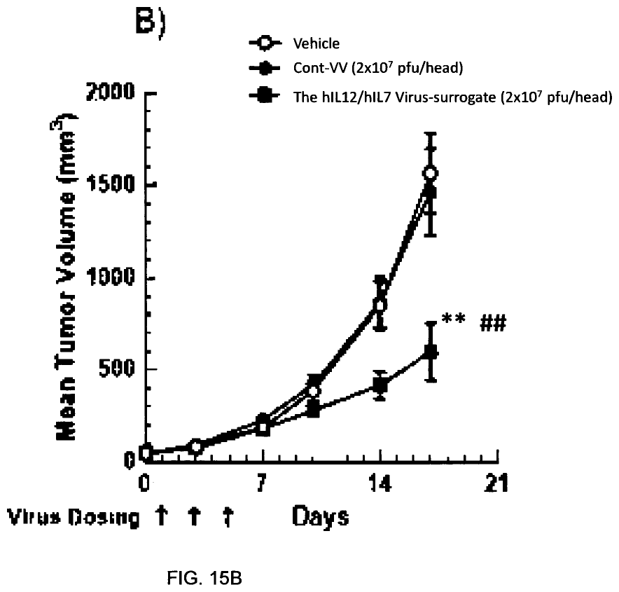

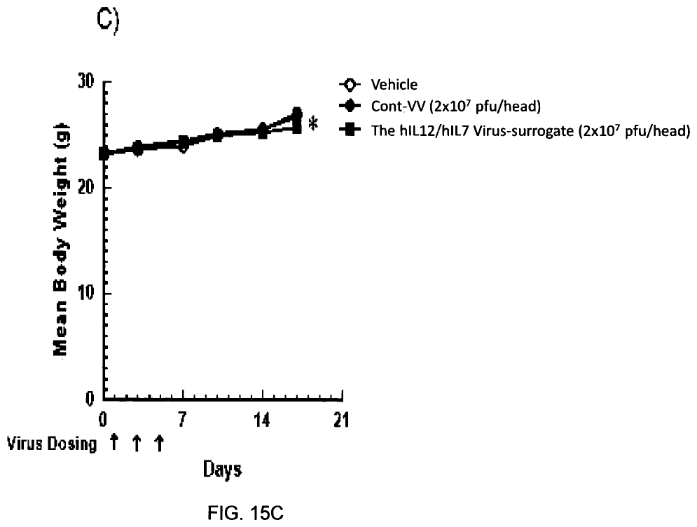

FIG. 15A is a graphs depicting tumor growth (tumor volume) in CT26.WT tumor cell bearing mice treated with the hIL12 and hIL7-carrying vaccinia virus-surrogate. Each point represents the mean±SEM (n=10). Cont-VV: recombinant vaccinia virus carrying no immune transgene; CT26.WT: murine colorectal carcinoma cell line; Vehicle: 30 mmol/L Tris-HCl containing 10% sucrose. *P<0.05, **P<0.01 versus the vehicle group (unpaired Student's t-test) #P<0.05, ##P<0.01 versus Cont-VV (unpaired Student's t-test).

FIG. 15B is a graphs depicting tumor growth (tumor volume) in CT26.WT tumor cell bearing mice treated with the hIL12 and hIL7-carrying vaccinia virus-surrogate. Each point represents the mean±SEM (n=10). Cont-VV: recombinant vaccinia virus carrying no immune transgene; CT26.WT: murine colorectal carcinoma cell line; Vehicle: 30 mmol/L Tris-HCl containing 10% sucrose. *P<0.05, **P<0.01 versus the vehicle group (unpaired Student's t-test) #P<0.05, ##P<0.01 versus Cont-VV (unpaired Student's t-test).

FIG. 15C is a graph depicting body weight changes in CT26.WT tumor cell bearing mice treated with the hIL12 and hIL7-carrying vaccinia virus-surrogate. Each point represents the mean±SEM (n=10). Cont-VV: recombinant vaccinia virus carrying no immune transgene; CT26.WT: murine colorectal carcinoma cell line; Vehicle: 30 mmol/L Tris-HCl containing 10% sucrose. *P<0.05, **P<0.01 versus the vehicle group (unpaired Student's t-test) #P<0.05, ##P<0.01 versus Cont-VV (unpaired Student's t-test).

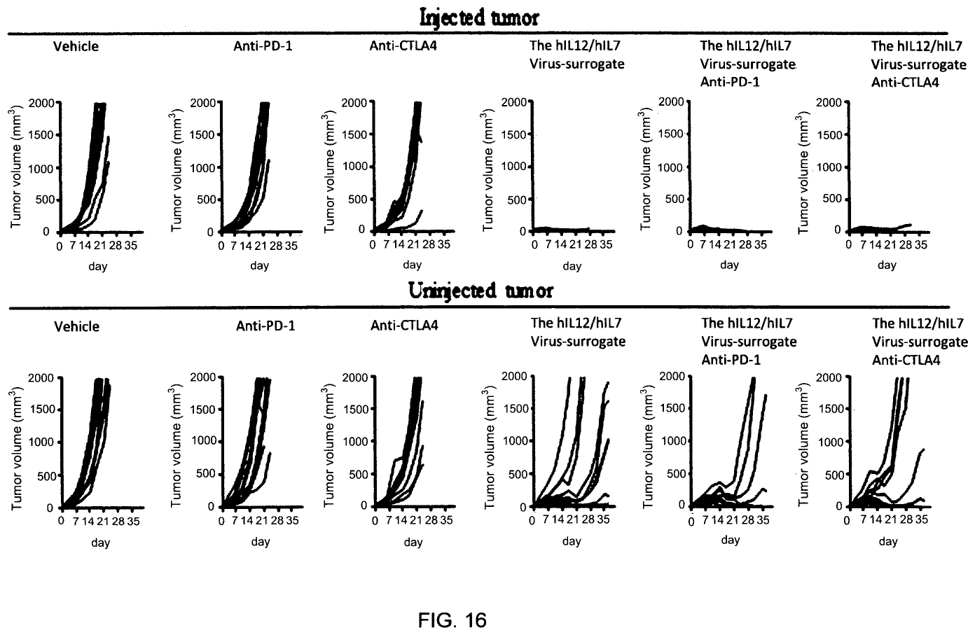

FIG. 16 depicts a series of graphs depicting tumor growth change (tumor volume) in bilaterally CT26.WT tumor-bearing mice treated with the hIL12 and hIL7-carrying vaccinia virus-surrogate with anti-PD-1 antibody or anti-CTLA4 antibody. Tumor volumes of individual mice are shown. Ab: antibody; : recombinant vaccinia virus carrying murine IL-12 gene and human IL-7 gene; CT26.WT: murine colorectal carcinoma cell line; IL-7: interleukin 7; IL-12: interleukin 12; Vehicle: 30 mmol/L Tris-HCl containing 10% sucrose.

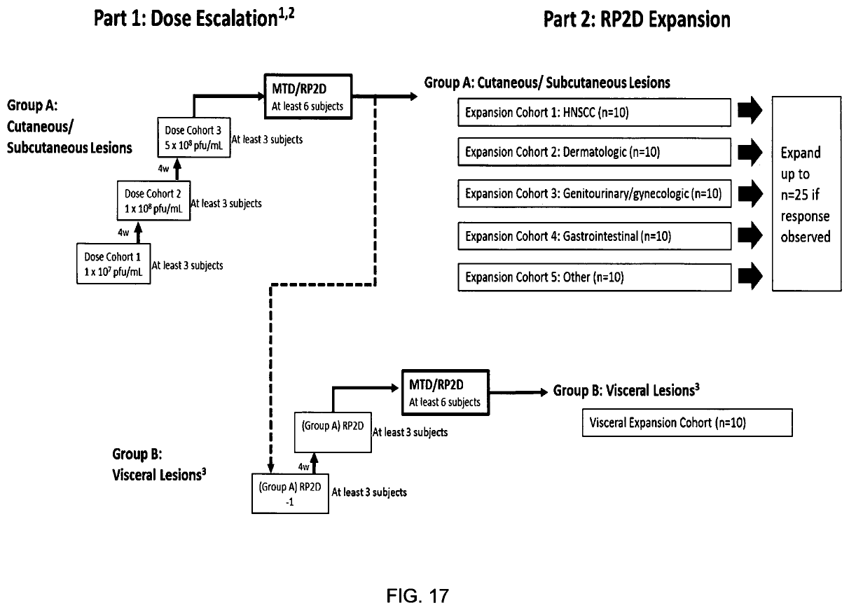

FIG. 17 depicts a First-In-Human (FIH) Phase I Study Schema. CT: computed tomography; DLT: dose-limiting toxicity; FIH: first-in-human; HNSCC: head and neck squamous cell carcinoma; MTD: maximum tolerated dose; n: number of patients in a specified cohort; RP2D: recommended phase 2 dose. ¹Proposed dose escalation levels. Actual dose escalation cohorts to be defined based on clinical data. ²≥4 weeks will elapse between completion of the DLT observation period for the previous cohort and the start of the next cohort. ³Enrollment in Group B dose escalation cohorts will/begin after MTD/RP2D in Group A.

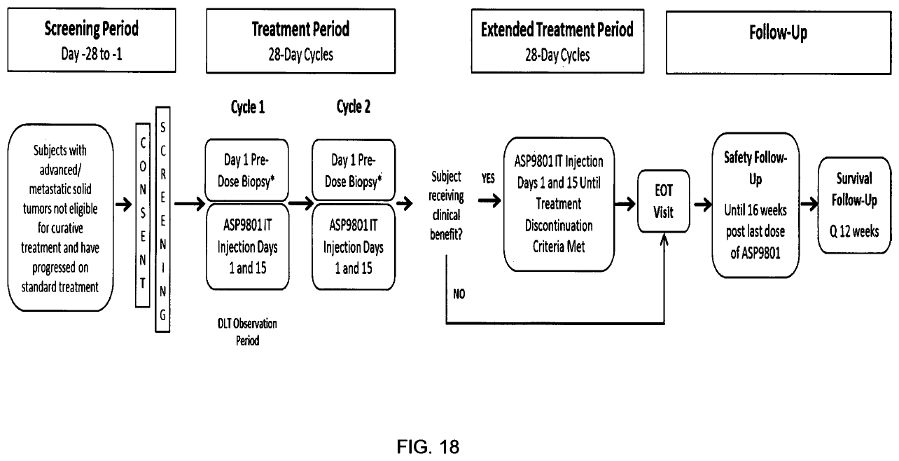

FIG. 18 depicts a First-In-Human (FIH) Phase I Study Visit Schema. DLT: dose limiting toxicity; EOT: end of treatment; FIH: first-in-human; IT: intratumoral; Q: every. * Cycle 1 predose biopsy may be performed up to 28 days prior to first injection. Cycle 2 predose biopsy may be taken up to 5 days prior to day 1 injection.

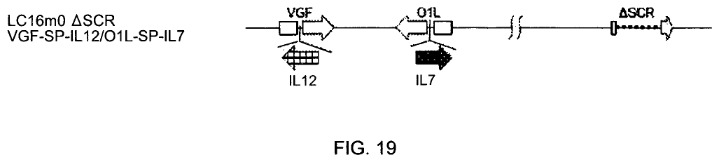

FIG. 19 schematically depicts the genome structure of a recombinant vaccinia virus, “LC16mO ΔSCR VGF-SP-IL12/O1L-SP-IL7,” also referred to as “hIL12 and hIL7-carrying vaccinia virus” or “hIL12/hIL7 virus”.

### DESCRIPTION OF EMBODIMENTS

### Detailed Description of the Invention

The present invention is based, at least in part, on the development of pharmaceutical compositions comprising an investigational oncolytic vaccinia virus and the discovery that such compositions are cytotoxic against various types of human cancer cell lines in vitro. The present invention is also based, at least in part, on the discovery that such pharmaceutical compositions have antitumor activity in vivo, that administration of the pharmaceutical compositions to a subject using a dosing regimen is very efficacious (e.g., the discovery that administration on days 1 and 15 is more efficacious as compared to a single administration), that administration of the pharmaceutical compositions to a subject induces intratumoral secretion of murine IL-12, human IL-7 and murine interferon gamma (IFN-γ) proteins and increased tumor infiltration with CD8+ T cells and CD4+ T cells, and that administration of the pharmaceutical compositions of the invention in combination with a checkpoint inhibitor, i.e., an anti-PD-1 antibody or an anti-CTLA4 antibody, induced higher antitumor activity than any of the treatments alone. The present invention is further based, at least in part, on the discovery that mice that achieved complete tumor regression (CR) following administration of the pharmaceutical compositions of the invention rejected the same cancer cells when re-challenged about 90 days after the CR, demonstrating establishment of antitumor immune memory. In addition, the present invention is based, at least in part, on the discovery that administration of the pharmaceutical compositions of the invention had an abscopal effect in a bilateral tumor model.

The following detailed description discloses how to make and use the present invention.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to.”

The term “or” is used herein to mean, and is used interchangeably with, the term “and/or,” unless context clearly indicates otherwise. The term “about” is used herein to mean within the typical ranges of tolerances in the art. For example, “about” can be understood as within about 2 standard deviations from the mean. In certain embodiments, about means +10%. In certain embodiments, about means +5%. When about is present before a series of numbers or a range, it is understood that “about” can modify each of the numbers in the series or range.

As used herein, the term “oncolytic virus” refers to a virus that selectively replicates in dividing cells (e.g., a proliferative cell such as a cancer cell) to slow the growth and/or lyse the dividing cell, either in vitro or in vivo, while having no or minimal replication in non-dividing cells. Typically, an oncolytic virus contains a viral genome packaged into a viral particle (or virion) and is infectious (i.e., capable of infecting and entering into a host cell or subject). As used herein, this term encompasses DNA and RNA vectors (depending on the virus in question) as well as viral particles generated thereof.

As used herein, the term vaccinia virus refers to a large, complex, enveloped virus belonging to the poxvirus family. Vaccinia viruses have a linear, double-stranded DNA genome approximately 190 kbp in length, which encodes approximately 250 genes. The dimensions of the virion are roughly 360×270×250 nm, with a mass of approximately 5-10 fg.

The terms “polypeptide”, “peptide” and “protein” refer to polymers of amino acid residues which comprise at least nine or more amino acids bonded via peptide bonds. The polymer can be linear, branched or cyclic and may comprise naturally occurring and/or amino acid analogs and it may be interrupted by non-amino acids. If the amino acid polymer is more than 50 amino acid residues, it is preferably referred to as a polypeptide or a protein whereas if it is 50 amino acids long or less, it is referred to as a “peptide”.

The terms “nucleic acid”, “nucleic acid molecule”, “polynucleotide” and “nucleotide sequence” are used interchangeably and define a polymer of any length of either polydeoxyribonucleotides (DNA) (e.g. cDNA, genomic DNA, plasmids, vectors, viral genomes, isolated DNA, probes, primers and any mixture thereof) or polyribonucleotides (e.g. mRNA, antisense RNA, siRNA) or mixed polyribo-polydeoxyribonucleotides. They encompass single or double-stranded, linear or circular, natural or synthetic, modified or unmodified polynucleotides. Moreover, a polynucleotide may comprise non-naturally occurring nucleotides and may be interrupted by non-nucleotide components.

An “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived.

In a general manner, the term “identity” refers to an amino acid to amino acid or nucleotide 5 to nucleotide correspondence between two polypeptide or nucleic acid sequences. The percentage of identity between two sequences is a function of the number of identical positions shared by the sequences, taking into account the number of gaps which need to be introduced for optimal alignment and the length of each gap. Various computer programs and mathematical algorithms are available in the art to determine the percentage of identity between amino acid sequences, such as for example the Blast program available at NCBI or ALIGN in Atlas of Protein Sequence and Structure (Dayhoffed, 1981, Suppl., 3: 482-9). Programs for determining identity between nucleotide sequences are also available in specialized data base (e.g. Genbank, the Wisconsin Sequence Analysis Package, BESTFIT, FASTA and GAP programs). For illustrative purposes, “at least 80% identity” means 80%, 81%, 82%, 83%, 84%, 85%, 86%, 87%, 88%, 89%, 90%, 91%, 92%, 93%, 94%, 95%, 96%, 97%, 98%, 99% or 100%.

The term “subject” generally refers to an organism for whom any product and method of the invention is needed or may be beneficial. Typically, the organism is a mammal, particularly a mammal selected from the group consisting of domestic animals, farm animals, sport animals, and primates. Preferably, the subject is a human who has been diagnosed as having or at risk of having a proliferative disease such as a cancer. The terms “subject” and “patients” may be used interchangeably when referring to a human organism and encompasses male and female.

### II. Pharmaceutical Compositions of the Invention

The present invention provides pharmaceutical compositions and formulations which include the oncolytic vaccinia viruses of the invention. Such pharmaceutical compositions are formulated based on the mode of delivery. In one example, the compositions are formulated for systemic administration via parenteral delivery, e.g., by intravenous (IV) delivery. In one embodiment, the compositions are formulated for intraperitoneal delivery. In another embodiment, the compositions are formulated for intratumoral delivery.

Accordingly, the present invention provides pharmaceutical compositions, e.g., pharmaceutical compositions suitable for intratumoral delivery, comprising about 1×10⁶ to about 1×10¹⁰ particle forming units (pfu)/ml of an oncolytic vaccinia virus, e.g., the hIL12/hIL7 virus, and a pharmaceutically acceptable carrier.

The phrase “pharmaceutically acceptable” refers to those compounds, materials, compositions, and/or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as sucrose, lactose, or glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as tromethamine, magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The pharmaceutical compositions of the invention may be in solution that is appropriate for human or animal use. The solvent or diluent of the solution may be isotonic, hypotonic or weakly hypertonic and has a relatively low ionic strength. Representative examples include sterile water, physiological saline (e.g. sodium chloride), Ringer's solution, glucose, trehalose or saccharose solutions, Hank's solution, and other aqueous physiologically balanced salt solutions (see for example the most current edition of Remington: The Science and Practice of Pharmacy, A. Gennaro, Lippincott, Williams&Wilkins).

In one embodiment, the pharmaceutical compositions of the invention are buffered for human use. Suitable buffers include without limitation phosphate buffer (e.g., PBS), bicarbonate buffer and/or Tris buffer, e.g., a buffer comprising tromethamine, capable of maintaining a physiological or slightly basic pH (e.g., from approximately pH 7 to approximately pH 9).

The pharmaceutical compositions of the invention may also contain other pharmaceutically acceptable excipients for providing desirable pharmaceutical or pharmacodynamic properties, including for example osmolarity, viscosity, clarity, color, sterility, stability, rate of dissolution of the formulation, modifying or maintaining release or absorption into an the human or animal subject, promoting transport across the blood barrier or penetration in a particular organ.

The pharmaceutical compositions of the invention may also comprise one or more adjuvant(s) capable of stimulating immunity (especially a T cell-mediated immunity) or facilitating infection of tumor cells upon administration, e.g. through toll-like receptors (TLR) such as TLR-7, TLR-8 and TLR-9, including without limitation alum, mineral oil emulsion such as, Freunds complete and incomplete (IFA), lipopolysaccharide or a derivative thereof (Ribi et al., 1986, Immunology and Immunopharmacology of Bacterial Endotoxins, Plenum Publ. Corp., NY, p 407-419), saponins such as QS21 (Sumino et al., 1998, J. Virol. 72: 4931; WO98/56415), imidazo-quinoline compounds such as Imiquimod (Suader, 2000, J. Am Acad Dermatol. 43:S6), S-27609 (Smorlesi, 2005, Gene Ther. 12: 1324) and related compounds such as those described in WO2007/147529, cytosine phosphate guanosine oligodeoxynucleotides such as CpG (Chu et al., 1997, J. Exp. Med. 186: 1623; Tritel et al., 2003, J. Immunol. 171: 2358) and cationic peptides such as IC-31 (Kritsch et al., 2005, J. Chromatogr Anal. Technol. Biomed. Life Sci. 822: 263-70).

In one embodiment, the pharmaceutical compositions of the invention are formulated to improve stability. For example, under the conditions of manufacture and long-term storage (i.e. for at least 6 months to two years) at freezing (e.g. −70° C., −20° C.), refrigerated (e.g. 4° C.) or ambient temperatures. The pharmaceutical compositions of the invention may be liquid or solid (e.g. dry powdered or lyophilized) obtained by a process involving, e.g., vacuum drying and freeze-drying.

In certain embodiments, the pharmaceutical compositions of the invention are formulated to ensure proper distribution or delayed release in vivo. For example, the pharmaceutical compositions may be formulated in liposomes. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g. J. Robinson in “Sustained and Controlled Release Drug Delivery Systems”, ed., Marcel Dekker, Inc., New York, 1978.

In one aspect, provided herein are pharmaceutical compositions comprising about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., the hIL12/hIL7 virus; and a pharmaceutically acceptable carrier. In one embodiment, the pharmaceutical composition is for intratumoral delivery.

In another aspect, provided herein are pharmaceutical compositions comprising about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., the hIL12/hIL7 virus; tromethamine at a concentration of about 10 mmol/L to about 50 mmol/L; and sucrose at a concentration of about 5% w/v to about 15% w/v, wherein the pH of the composition is about 5.0 to about 8.5. In one embodiment, the pharmaceutical composition is for intratumoral delivery.

The pharmaceutical compositions containing the oncolytic vaccinia viruses of the invention, e.g., the hIL12/hIL7 virus, are useful for treating a subject having a cancer.

The pharmaceutical compositions of the invention may include about 1×10⁶ to about 1×10¹⁰, about 1×10⁷ to about 1×10⁹, about 1×10⁷, about 5×10⁷, about 1×10⁸, about 5×10⁸, about 1×10⁹, or about 5×10⁹ particle forming units (pfu)/ml of the oncolytic vaccinia virus of the invention, e.g., the hIL12/hIL7 virus. Values intermediate to the above recited ranges and values are also intended to be part of this invention. In addition, ranges of values using a combination of any of the above recited values as upper and/or lower limits are intended to be included.

In some embodiments, the pharmaceutical compositions of the invention include tromethamine (Tris-HCl). The concentration of tromethamine in the pharmaceutical compositions of the invention may be about 10 mmol/L to about 50 mmol/L; about 15 mmol/L to about 45 mmol/L; 20 mmol/L to about 40 mmol/L; 25 mmol/L to about 35 mmol/L; or about 30 mmol/L. Values intermediate to the above recited ranges and values are also intended to be part of this invention. In addition, ranges of values using a combination of any of the above recited values as upper and/or lower limits are intended to be included.

In other embodiments, the pharmaceutical compositions of the invention include a sugar, such as sucrose. The concentration of sucrose in the pharmaceutical compositions of the invention may be about 5% w/v to about 15% w/v, about 6% w/v to about 14% w/v; about 7% w/v to about 13% w/v; about 8% w/v to about 12% w/v; about 9% w/v to about 11% w/v; or about 10% w/v of sucrose. Values intermediate to the above recited ranges and values are also intended to be part of this invention. In addition, ranges of values using a combination of any of the above recited values as upper and/or lower limits are intended to be included.

In one embodiment, the pharmaceutical compositions of the invention are preservative-free. In another embodiment of the invention, the pharmaceutical compositions of the invention include a preservative.

The pH of the pharmaceutical compositions of the invention may be between about 5.0 to about 8.5, about 5.5 to about 8.5, about 6.0 to about 8.5, about 6.5 to about 8.5, about 7.0 to about 8.5, about 5.0 to about 8.0, about 5.5 to about 8.0, about 6.0 to about 8.0, about 6.5 to about 8.0, about 7.0 to about 8.0, about 6.5 to about 8.5, about 7.5 to about 8.5, about 7.5 to about 8.0, about 6.8 to about 7.8, or about 7.6. Ranges and values intermediate to the above recited ranges and values are also intended to be part of this invention. In addition, ranges of values using a combination of any of the above recited values as upper and/or lower limits are intended to be included.

The pharmaceutical compositions of the invention are physically and chemically stable.

As used herein, the term “stable” refers to a pharmaceutical composition and/or an oncolytic vaccinia virus within such a pharmaceutical composition which essentially retains its physical stability and/or chemical stability and/or biological activity. Various analytical techniques for measuring stability of the composition and the dsRNA agent therein are available in the art and are described herein.

A pharmaceutical composition “retains its physical stability” if it shows substantially no signs of, e.g., increased impurities upon visual examination or UV examination of color and/or clarity, or as measured by, for example HPLC analysis, e.g., denaturing IP RP-HPLC, non-dentauring IP RP-HPLC, and/or denaturing AX-HPLC analysis.

An oncolytic vaccinia virus “retains its chemical stability” in a pharmaceutical composition, if the chemical stability at a given time is such that the oncolytic vaccinia virus is considered to still retain its biological activity.

An oncolytic vaccinia virus “retains its biological activity” in a pharmaceutical composition, if the oncolytic vaccinia virus in a composition is biologically active for its intended purpose.

In some embodiments, the compositions of the invention are stable for at least about 6 months to about 2 years when stored at about −70° C.

### III. Oncolytic Vaccinia Viruses for Use in the Pharmaceutical Compositions of the Invention

Suitable oncolytic vaccinia viruses for use in the present invention are described in U.S. Patent Publication No. 2017/0340687, the entire contents of which are incorporated herein by reference. Such oncolytic vaccinia viruses include a polynucleotide encoding IL-7; and a polynucleotide encoding IL-12. FIG. 19 schematically depicts the genome structure of a recombinant vaccinia virus, “LC16mO ΔSCR VGF-SP-IL12/O1L-SP-IL7,” also referred to as “hIL12 and hIL7-carrying vaccinia virus” or “hIL12/hIL7 virus”.

Suitable vaccinia viruses for use in the present invention are derived from the genus Orthopoxvirus in the family Poxviridae. Strains of the vaccinia virus used in the present invention include, but not limited to, the strains Lister, New York City Board of Health (NYBH), Wyeth, Copenhagen, Western Reserve (WR), Modified Vaccinia Ankara (MVA), EM63, Ikeda, Dalian, Tian Tan, and the like. The strains Lister and MVA are available from American Type Culture Collection (ATCC VR-1549 and ATCC VR-1508, respectively).

Vaccinia virus strains established from these strains may be used in the present invention. For example, the strains LC16, LC16m8, and LC16mO established from the strain Lister may be used in the present invention. The strain LC16mO is a strain generated via the strain LC16 by subculturing at low temperature the Lister strain as the parent strain. The LC16m8 strain is a strain generated by further subculturing at low temperature the strain LC16mO, having a frameshift mutation in the B5R gene, a gene encoding a viral membrane protein, and attenuated by losing the expression and the function of this protein (Tanpakushitsu kakusan koso (Protein, Nucleic acid, Enzyme), 2003, vol. 48, p. 1693-1700).

The whole genome sequences of the strains Lister, LC16m8, and LC16mO are known and may be found in, for example, GenBank Accession Nos. AY678276.1, AY678275.1, and, AY678277.1, respectively, the entire contents of each of which are incorporated herein by reference. Therefore, the strains LC16m8 and LC16mO can be made from the strain Lister by a known technique, such as homologous recombination or site-directed mutagenesis.

In one embodiment, a vaccinia virus for use in the present invention is the strain LC16mO.

IL-7 is a secretory protein functioning as an agonist for the IL-7 receptor. IL-7 contributes to the survival, proliferation, and differentiation of T cells, B cells, or the like (Current Drug Targets, 2006, vol. 7, p. 1571-1582). In the present invention, IL-7 encompasses IL-7 occurring naturally and modified forms having the function thereof. In one embodiment, IL-7 is human IL-7. In the present invention, human IL-7 encompasses human IL-7 occurring naturally and modified forms having the function thereof. In one embodiment, human IL-7 is selected from the group consisting of:

a polypeptide comprising the amino acid sequence set forth in Accession No. NP_000871.1 (the entire contents of which is incorporated herein by reference);

a polypeptide consisting of an amino acid sequence in which 1 to 10 amino acids are deleted from, substituted in, inserted into, and/or added to the amino acid sequence set forth in Accession No. NP_000871.1 (the entire contents of which is incorporated herein by reference), and having the function of human IL-7; and

a polypeptide comprising an amino acid sequence having about 85, 86, 87, 88, 89, 90, 91, 92, 93, 94, 95, 96, 97, 98, 99, or about 100% nucleotide identity to the entire amino acid sequence set forth in GenBank Accession No. NP_000871.1 (the entire contents of which is incorporated herein by reference), and having the function of human IL-7.

In relation with this, the function of human IL-7 refers to the effect on the survival, proliferation, and differentiation of human immune cells.

Human IL-7 used in the present invention is preferably a polypeptide consisting of the amino acid sequence set forth in GenBank Accession No. NP_000871.1 (the entire contents of which is incorporated herein by reference).

IL-12 is a heterodimer of the IL-12 subunit p40 and the IL-12 subunit a. IL-12 has been reported to have the function of activating and inducing the differentiation of T cells and NK cells (Cancer Immunology Immunotherapy, 2014, vol. 63, p. 419-435). In the present invention, IL-12 encompasses IL-12 occurring naturally and modified forms having the function thereof. In one embodiment, IL-12 is human IL-12. In the present invention, human IL-12 encompasses human IL-12 occurring naturally and modified forms having the function thereof. In one embodiment, human IL-12 is selected, as a combination of the human IL-12 subunit p40 (a) and the human IL-12 subunit a (b), from the group consisting of (1-3):

(1) (a) polypeptides comprising a polypeptide comprising the amino acid sequence set forth in GenBank Accession No. NP_002178.2 (the entire contents of which is incorporated herein by reference); a polypeptide consisting of an amino acid sequence in which 1 to 10 amino acids are deleted from, substituted in, inserted into, and/or added to the amino acid sequence set forth in GenBank Accession No. NP_002178.2 (the entire contents of which is incorporated herein by reference); or a polypeptide comprising an amino acid sequence having about 85, 86, 87, 88, 89, 90, 91, 92, 93, 94, 95, 96, 97, 98, 99, or about 100% nucleotide identity to the entire amino acid sequence set forth in GenBank Accession No. NP_002178.2 (the entire contents of which is incorporated herein by reference); and

(1) (b) a polypeptide comprising the amino acid sequence set forth in GenBank Accession No. NP_000873.2 (the entire contents of which are incorporated herein by reference); a polypeptide consisting of an amino acid sequence in which 1 to 10 amino acids are deleted from, substituted in, inserted into, and/or added to the amino acid sequence set forth in GenBank Accession No. NP_000873.2 (the entire contents of which are incorporated herein by reference); or a polypeptide comprising an amino acid sequence having about 85, 86, 87, 88, 89, 90, 91, 92, 93, 94, 95, 96, 97, 98, 99, or about 100% nucleotide identity to the entire amino acid sequence set forth in GenBank Accession No. NP_002178.2 (the entire contents of which is incorporated herein by reference), and having the function of human IL-12;

(2) (a) polypeptides comprising a polypeptide consisting of the amino acid sequence set forth in GenBank Accession No. NP_002178.2 (the entire contents of which is incorporated herein by reference), and

(2) (b) a polypeptide comprising the amino acid sequence set forth in GenBank Accession No. NP_000873.2 (the entire contents of which are incorporated herein by reference); a polypeptide consisting of an amino acid sequence in which 1 to 10 amino acids are deleted from, substituted in, inserted into, and/or added to the amino acid sequence set forth in GenBank Accession No. NP_000873.2 (the entire contents of which are incorporated herein by reference); or a polypeptide comprising an amino acid sequence having about 85, 86, 87, 88, 89, 90, 91, 92, 93, 94, 95, 96, 97, 98, 99, or about 100% nucleotide identity to the entire amino acid sequence set forth in GenBank Accession No. NP_000873.2 (the entire contents of which are incorporated herein by reference), and having the function of human IL-12; and

(3) (a) a polypeptide comprising a polypeptide comprising the amino acid sequence set forth in GenBank Accession No. NP_002178.2 (the entire contents of which is incorporated herein by reference); a polypeptide consisting of an amino acid sequence in which 1 to 10 amino acids are deleted from, substituted in, inserted into, and/or added to the amino acid sequence set forth in GenBank Accession No. NP_002178.2 (the entire contents of which is incorporated herein by reference); or a polypeptide comprising an amino acid sequence having about 85, 86, 87, 88, 89, 90, 91, 92, 93, 94, 95, 96, 97, 98, 99, or about 100% nucleotide identity to the entire amino acid sequence set forth in or more identity with the amino acid sequence set forth in GenBank Accession No. NP_002178.2 (the entire contents of which is incorporated herein by reference); and

(3) (b) a polypeptide consisting of the amino acid sequence set forth in GenBank Accession No. NP_000873.2 (the entire contents of which are incorporated herein by reference), and having the function of human IL-12.

In relation with this, the function of human IL-12 refers to activating and/or differentiating effects on T cells or NK cells. The IL-12 subunit p40 and the IL-12 subunit a can form IL-12 by direct binding. Moreover, the IL-12 subunit p40 and the IL-12 subunit a can be conjugated via a linker.

Human IL-12 used in the present invention is preferably a polypeptide comprising a polypeptide consisting of the amino acid sequence set forth in GenBank Accession No. NP_002178.2 (the entire contents of which is incorporated herein by reference) and a polypeptide consisting of the amino acid sequence set forth in GenBank Accession No. NP_000873.2 (the entire contents of which are incorporated herein by reference).

As used herein, “identity” means the value Identity obtained by a search using the NEEDLE program (Journal of Molecular Biology, 1970, vol. 48, p. 443-453) with the default parameters. The parameters are as follows:

Gap penalty=10

Extend penalty=0.5

Matrix=EBLOSUM62

The polynucleotides encoding IL-7 and IL-12 can be synthesized based on publicly available sequence information using a method of polynucleotide synthesis known in the field. Moreover, once the polynucleotides are obtained; then modified forms having the function of each polypeptide can be generated by introducing mutation into a predetermined site using a method known by those skilled in the art, such as site-directed mutagenesis (Current Protocols in Molecular Biology edition, 1987, John Wiley & Sons Sections 8.1-8.5).

The polynucleotides each encoding IL-7 and IL-12 can be introduced into vaccinia virus by a known technique, such as homologous recombination or site-directed mutagenesis. For example, a plasmid (also referred to as transfer vector plasmid DNA) in which the polynucleotide(s) is (are) introduced into the nucleotide sequence at the site desired to be introduced can be made and introduced into cells infected with vaccinia virus. The region in which the polynucleotides each encoding IL-7 and IL-12, foreign genes, are introduced is preferably a gene region that is inessential for the life cycle of vaccinia virus. For example, in a certain aspect, the region in which IL-7 and/or IL-12 is (are) introduced may be a region within the VGF gene in vaccinia virus deficient in the VGF function, a region within the O1L gene in vaccinia virus deficient in the O1L function, or a region or regions within either or both of the VGF and O1L genes in vaccinia virus deficient in both VGF and O1L functions. In the above, the foreign gene(s) can be introduced so as to be transcribed in the direction same as or opposite to that of the VGF and O1L genes.

Methods for introducing transfer vector plasmid DNA into cells are not limited, but examples of methods that can be used include the calcium phosphate method and electroporation.

When introducing the polynucleotides each encoding IL-7 and IL-12, which are foreign genes, a suitable promoter(s) can be operably linked in the upstream of the foreign gene(s). In this way, the foreign gene(s) in the vaccinia virus according to the present invention, the vaccinia virus to be used in combination, or the vaccinia viruses for the combination kit can be linked to a promoter that can promote expression in tumor cells. Examples of such a promoter include PSFJ1-10, PSFJ2-16, the p7.5K promoter, the p11K promoter, the T7.10 promoter, the CPX promoter, the HF promoter, the H6 promoter, and the T7 hybrid promoter.

A vaccinia virus for use in the present invention can include attenuated and/or tumor-selective vaccinia viruses.

As used herein, “attenuated” means low toxicity (for example, low cytolosis) to normal cells (for example, non-tumor cells).

As used herein, “tumor selective” means toxicity to tumor cells (for example, oncolytic) higher than that to normal cells (for example, non-tumor cell).

Vaccinia viruses genetically modified to be deficient in the function of a specific protein or to suppress the expression of a specific gene or protein (Expert Opinion on Biological Therapy, 2011, vol. 11, p. 595-608) may be used in the present invention.

For example, to enhance the tumor selectivity of the vaccinia virus, the following can be performed: the deletion of thymidine kinase (TK) (Cancer Gene Therapy, 1999, Vol. 6, p. 409-422); the introduction of a modified TK gene, a modified Hemagglutinin (HA) gene, and a modified F3 gene or an interrupted F3 genetic locus (International Publication No. 2005/047458); the deletion of function of TK, HA, and F14.5L (Cancer Research, 2007, Vol. 67, p. 10038-10046); the deletion of function of TK and B18R (PLoS Medicine, 2007, Vol. 4, p. e353); the deletion of function of TK and a ribonucleotide reductase (PLoS Pathogens, 2010, Vol. 6, p. e1000984); the deletion of function of SPI-1 and SPI-2 (Cancer Research, 2005, Vol. 65, p. 9991-9998); the deletion of function of SPI-1, SPI-2 and TK (Gene Therapy, 2007, Vol. 14, p. 638-647) or the introduction of mutations into the E3L and K3L regions (International Publication No. 2005/007824). Moreover, the A34R region (Molecular Therapy, 2013, Vol. 21, p. 1024-1033) can be deleted in expectation of attenuating the removal of virus by the neutralization effect of an anti-vaccinia virus antibody in the living body. Moreover, the interleukin-1b (IL-1b) receptor can be deleted (International Publication No. 2005/030971) in expectation of the activation of immune cells by the vaccinia virus.

The aforementioned insertion of a foreign gene or deletion or mutation of a gene can be achieved by a well-known homologous recombination method or site-specific mutagenesis. The vaccinia virus of the present invention may have a combination of the aforementioned genetic modifications.

As used herein, the term “lacking” means that the genetic region specified by this term is not functioning or that the genetic region specified by this term has been deleted. For example, with regard to the “lacking,” deletion may have occurred in a region that is a specified genetic region or in a genetic region surrounding a specified genetic region.

A suitable oncolytic vaccinia virus of the present invention may comprise a deletion in the gene encoding B5R.

B5R is a type 1 membrane protein of a vaccinia virus. When the virus proliferates within cells and spreads to near-by cells or other sites within the host, B5R increases the efficiency thereof. The B5R includes a B5R having an amino acid sequence set forth in GenBank Accession No. AAA48316.1 (the entire contents of which are incorporated herein by reference). The B5R has a signal peptide, a region referred to as four SCR domains (SCR domains 1-4), a region referred to as stalk, a transmembrane domain and a cytoplasmic tail, sequentially from the N-terminal side toward the C-terminal side.

More specifically, in B5R, the signal peptide is a region of B5R corresponding to the 1st amino acid through the 19th amino acid of an amino acid sequence set forth in GenBank Accession No. AAA48316.1; SCR domains 1-4 is a region of B5R corresponding to the 20th amino acid through the 237th amino acid of an amino acid sequence set forth in GenBank Accession No. AAA48316.1; the stalk is a region of B5R corresponding to the 238th amino acid through the 275th amino acid of an amino acid sequence set forth in GenBank Accession No. AAA48316.1; the transmembrane domain is a region of B5R corresponding to the 276th amino acid through the 303th amino acid of an amino acid sequence set forth in GenBank Accession No. AAA48316.1; and the cytoplasmic tail is a region of B5R corresponding to the 304th amino acid through the 317th amino acid of an amino acid sequence set forth in GenBank Accession No. AAA48316.1 (Journal of Virology, 2005, Vol. 79, p. 6260-6271).

As used herein, the term “corresponding” is not limited to the concept of having an amino acid sequence that matches an amino acid sequence specified by this term completely and accurately but includes the concept of having amino acid sequences that are altered from an amino acid sequence specified by this term (e.g., deletion, substitution, insertion and/or addition of amino acid), due to a method for analyzing the function of protein, difference in vaccinia virus strains and what not. Those skilled in the art can identify the gene of B5R and each region of B5R in each of those different vaccinia virus strains, on the basis of the aforementioned amino acid sequence. When B5R is expressed on the external membrane, the signal peptide has already been removed, and SCR domains 1-4 and the stalk have been exposed on the external membrane of EEV (Journal of Virology, 1998, Vol. 72, p. 294-302). As used herein, a region consisting of SCR domains 1-4 and the stalk is sometimes referred to as the “extracellular region.”

As indicated above, a suitable oncolytic vaccinia virus of the present invention may comprise a deletion in the gene encoding B5R. In one embodiment, a suitable oncolytic vaccinia virus of the present invention includes a gene encoding SCR domain deleted B5R.

As used herein, the term “gene encoding SCR domain-deleted B5R” refers to a gene encoding B5R that has SCR domains 1-4 deleted fully or partially and thereby lacking the function thereof.

A suitable method for determining whether or not the function of B5R has been removed in a vaccinia virus includes a method for confirming whether or not the ability to avoid neutralization against an neutralizing antibody targeting B5R is increased, as compared with an vaccinia virus whose SCR domains have not been deleted.

In one embodiment, SCR domain-deleted B5R has the extracellular region of B5R other than the deleted-region.

In one embodiment, SCR domain-deleted B5R has the extracellular region of B5R other than the deleted-region, and the transmembrane domain.

In one embodiment, SCR domain-deleted B5R has the extracellular region of B5R other than the deleted-region, the transmembrane domain and the cytoplasmic tail.

In one embodiment, SCR domain-deleted B5R has the stalk. In one embodiment, SCR domain-deleted B5R has the stalk and the transmembrane domain.

In one embodiment, SCR domain-deleted B5R has the stalk, the transmembrane domain and the cytoplasmic tail.

In one embodiment, the vaccinia virus of the present invention can present B5R, which has the extracellular region with SCR domains 1-4 deleted fully or partially on the surface of the virus, when it is in the form of EEV.

In one embodiment, the term “SCR domain-deleted B5R” in the vaccinia virus of the present invention is B5R having four SCR domains (SCR domains 1-4) deleted.

As used herein, the term “deletion of SCR domains 1-4” or any expression similar thereto, which is described in the context of four SCR domains, is not limited to the complete and accurate deletion of the region constituted of SCR domains 1-4 but includes the concept that one, two or three amino acids at the terminal of the aforementioned region remains in B5R. The deletion of SCR domains 1-4 in the vaccinia virus of the present invention includes the deletion of the B5R region corresponding to amino acid residues 22-237 of the amino acid sequence set forth in GenBank Accession No. AAA48316.1. The amino acid sequence of GenBank Accession No. AAA48316.1 is set forth in SEQ ID NO: 1.

In one embodiment, B5R having SCR domains 1-4 deleted contains the extracellular region of B5R.

In one embodiment, B5R having SCR domains 1-4 deleted contains the extracellular region of B5R and the transmembrane domain.

In one embodiment, B5R having SCR domains 1-4 deleted contains the extracellular region of B5R, the transmembrane domain and the cytoplasmic tail.

In one embodiment, B5R having SCR domains 1-4 deleted contains the stalk.

In one embodiment, B5R having SCR domains 1-4 deleted contains the stalk and the transmembrane domain.

In one embodiment, B5R having SCR domains 1-4 deleted contains the stalk, the transmembrane domain and the cytoplasmic tail.

In one embodiment, the vaccinia virus of the present invention can present B5R, which has the extracellular region with SCR domains 1-4 deleted fully or partially on the surface of the virus, when it is in the form of EEV.

In one embodiment, SCR domain-deleted B5R contains the region of B5R corresponding to amino acid residues 238-275 of the amino acid sequence set forth in GenBank Accession No. AAA48316.1 (amino acid residues 22-59 of the amino acid sequence in SEQ ID NO: 2).

In one embodiment, SCR domain-deleted B5R contains the region of B5R corresponding to amino acid residues 238-303 of the amino acid sequence set forth in GenBank Accession No. AAA48316.1 (amino acid residues 22-87 of the amino acid sequence set forth in SEQ ID NO: 2).

In one embodiment, SCR domain-deleted B5R contains the region of B5R corresponding to amino acid residues 238-317 of the amino acid sequence set forth in GenBank Accession No. AAA48316.1 (amino acid residues 22-101 of the amino acid sequence set forth in SEQ ID NO: 2).

In one embodiment, the gene encoding SCR domain-deleted B5R in the vaccinia virus of the present invention encodes the signal peptide of B5R.

In one embodiment, the gene encoding SCR domain-deleted B5R encodes a polypeptide containing the signal peptide of B5R and the extracellular region of B5R.

In one embodiment, the gene encoding SCR domain-deleted B5R encodes a polypeptide containing the signal peptide of B5R, the extracellular region of B5, and the transmembrane domain.

In one embodiment, the gene encoding SCR domain-deleted B5R encodes a polypeptide containing the signal peptide of B5R, the extracellular region of B5R, the transmembrane domain, and the cytoplasmic tail.

In one embodiment, the gene encoding SCR domain-deleted B5R encodes a polypeptide containing the signal peptide and stalk of B5R.

In one embodiment, the gene encoding SCR domain-deleted B5R encodes a polypeptide containing the signal peptide, stalk, and transmembrane domain of B5R.

In one embodiment, B5R having SCR domains 1-4 deleted encodes a polypeptide substantially containing the signal peptide of B5R, the extracellular region of B5R, the transmembrane domain, and the cytoplasmic tail.

In one embodiment, B5R having SCR domains 1-4 deleted encodes a polypeptide substantially containing the signal peptide, stalk, transmembrane domain and cytoplasmic tail of B5R.

As used herein, the term “substantially containing” means that this term contains elements specified by this term and that if other elements are contained, those elements neither block the activity or action of the listed elements disclosed by the present invention nor contribute to such activity or action. By way of example, the form in which one to several amino acids have been added or deleted is one of forms specified by the term “substantially containing.”

Examples of the signal peptide of B5R include the region of B5R corresponding to amino acid residues 1-19 of the amino acid sequence set forth in GenBank Accession No. AAA48316.1 (amino acid residues 1-19 of the amino acid sequence set forth in SEQ ID NO: 2).

Examples of the stalk of B5R include the region of B5R corresponding to amino acid residues 238-275 of the amino acid sequence set forth in GenBank Accession No. AAA48316.1 (amino acid residues 22-59 of the amino acid sequence set forth in SEQ ID NO: 2).

Examples of the transmembrane domain of B5R include the region of B5R corresponding to amino acid residues 276-303 of the amino acid sequence set forth in GenBank Accession No. AAA48316.1 (amino acid residues 60-87 of the amino acid sequence set forth set forth in SEQ ID NO: 2).

Examples of the cytoplasmic tail of B5R include the region of B5R corresponding to amino acid residues 304-317 of the amino acid sequence set forth in GenBank Accession No. AAA48316.1 (amino acid residues 88-101 of the amino acid sequence set forth in SEQ ID NO: 2).

In one embodiment, a gene encoding SCR domain-deleted B5R encodes the signal peptide of B5R corresponding to amino acid residues 1-19 of the amino acid sequence set forth in SEQ ID NO: 2.

In one embodiment, a gene encoding SCR domain-deleted B5R encodes the signal peptide of B5R having an amino acid sequence of amino acid residues 1-19 of the amino acid sequence set forth in SEQ ID NO: 2.

In one embodiment, a gene encoding SCR domain-deleted B5R encodes a polypeptide containing the signal peptide of B5R corresponding to amino acid residues 1-19 of the amino acid sequence set forth in SEQ ID NO: 2 and the stalk of B5R corresponding to amino acid residues 22-59 of the amino acid sequence set forth in SEQ ID NO: 2.

In one embodiment, a gene encoding SCR domain-deleted B5R encodes a polypeptide containing the signal peptide of B5R having an amino acid sequence of amino acid residues 1-19 of the amino acid sequence set forth in SEQ ID NO: 2 and the stalk of B5R having an amino acid sequence of amino acid residues 22-59 of the amino acid sequence set forth in SEQ ID NO: 2.

In one embodiment, a gene encoding SCR domain-deleted B5R encodes a polypeptide containing the signal peptide of B5R corresponding to an amino acid sequence of amino acid residues 1-19 of the amino acid sequence set forth in SEQ ID NO: 2, the stalk of B5R corresponding to an amino acid sequence of amino acid residues 22-59 of the amino acid sequence set forth in SEQ ID NO: 2 and the transmembrane domain of B5R corresponding to an amino acid sequence of amino acid residues 60-87.of the amino acid sequence set forth in SEQ ID NO: 2.

In one embodiment, a gene encoding SCR domain-deleted B5R encodes a polypeptide containing the signal peptide of B5R having an amino acid sequence of amino acid, residues 1-19 of the amino acid sequence set forth in SEQ ID NO: 2, the stalk of B5R having an amino acid sequence of amino acid residues 22-59 of the amino acid sequence set forth in SEQ ID NO: 2 and the transmembrane domain of B5R having an amino acid sequence of amino acid residues 60-87 of the amino acid sequence set forth in SEQ ID NO: 2.

In one embodiment, a gene encoding SCR domain-deleted B5R encodes a polypeptide having an amino acid sequence of B5R corresponding to the amino acid sequence set forth in SEQ ID NO: 2. In one embodiment, a gene encoding SCR domain-deleted B5R encodes a polypeptide having the amino acid sequence set forth in SEQ ID NO: 2.

A well-known method can be used to determine whether or not the vaccinia virus of the present invention encodes B5R having SCR domains 1-4 detected fully or partially. By way of example, it can be determined by confirming the presence of SCR domains 1-4 by a immunochemical method using an antibody that binds SCR domains 1-4 for B5R expressed on the surface of an vaccinia virus, or determining the presence or size of the region encoding the SCR domains 1-4 using polymerase chain reaction (PCR).

Suitable oncolytic vaccinia viruses of the present invention may be deficient in the function of O1L may be used (Journal of Virology, 2012, vol. 86, p. 2323-2336).

In addition, in order to reduce the clearance of virus by the neutralization effect of anti-vaccinia virus antibodies in the living body, vaccinia virus deficient in the extracellular region of B5R (Virology, 2004, vol. 325, p. 425-431) or vaccinia virus deficient in the A34R region (Molecular Therapy, 2013, vol. 21, p. 1024-1033) may be used.

Furthermore, in order to activate immune cells by the vaccinia virus, vaccinia virus deficient in interleukin-1β (IL-1β) receptor, as described in PCT Publication No. WO 2005/030971 (the entire contents of which are incorporated herein by reference) may be used. Such insertion of a foreign gene or deletion or mutation of a gene can be made, for example, by a known homologous recombination or site-directed mutagenesis.

Moreover, vaccinia virus having a combination of such genetic modifications may be used in the present invention.

As used herein, “being deficient” means that the gene region specified by this term has no function and used in a meaning including deletion of the gene region specified by this term. For example, “being deficient” may be a result of the deletion in a region consisting of the specified gene region or the deletion in a neighboring gene region comprising the specified gene region.

In one embodiment, the vaccinia virus for use in the present invention is deficient in the function of VGF.

In one embodiment, the vaccinia virus for use in the present invention is deficient in the function of O1L.

In one embodiment, the vaccinia virus for use in the present invention is deficient in the functions of VGF and O1L.

The function of VGF and/or O1L may be made deficient in vaccinia virus based on the method described in PCT Publication No. WO 2015/076422, the entire contents of which are incorporated herein by reference.

VGF is a protein having a high amino acid sequence homology with epidermal growth factor (EGF), binds to the epidermal growth factor receptor like EGF, and activates the signal cascade from Ras, Raf, Mitogen-activated protein kinase (MAPK)/the extracellular signal-regulated kinase (ERK) kinase (MAPK/ERK kinase, MEK), and to following ERK to promote the cell division.

O1L maintains the activation of ERK and contributes to the cell division along with VGF.

Being “deficient in the function of VGF and/or O1L of vaccinia virus” refers to loss of the expression of the gene encoding VGF and/or the gene encoding O1L or the normal function of VGF and/or O1L when expressed. The deficiency in the function of VGF and/or O1L of vaccinia virus may be caused by the deletion of all or a part of the gene encoding VGF and/or the gene encoding O1L. Moreover, the genes may be mutated by nucleotide substitution, deletion, insertion, or addition to prevent the expression of normal VGF and/or O1L. Moreover, a foreign gene may be inserted in the gene encoding VGF and/or the gene encoding O1L. In the present invention, when the normal gene product is not expressed due to a mutation such as the substitution, deletion, insertion or addition of a gene, it is referred to as the lacking of the gene.

In one embodiment, the vaccinia virus used in the present invention is an LC16mO strain vaccinia virus lacking the function of VGF and O1L.

As used herein, a gene is “deficient” when the normal product of the gene is not expressed by mutation such as genetic substitution, deletion, insertion, or addition.

Whether or not the vaccinia virus according to the present invention, is deficient in the function of VGF and/or O1L may be determined with a known method, for example, by evaluating the function of VGF and/or O1L, testing for the presence of VGF or O1L by an immunochemical technique using an antibody against VGF or an antibody against O1L, or determining the presence of the gene encoding VGF or the gene encoding O1L by the polymerase chain reaction (PCR).

The aforementioned insertion of a foreign gene or deletion or mutation of a gene can be achieved by a well-known homologous recombination method or site-specific mutagenesis. In the present invention, a vaccinia virus having a combination of the aforementioned genetic modifications can be used.

In certain embodiments of the invention, in addition to including a polynucleotide encoding IL-7; and a polynucleotide encoding IL-12, the oncolytic vaccinia viruses of the invention include a gene encoding B5R lacking the function of VGF and O1L and having SCR domains deleted. In this embodiment, the SCR domain-deleted B5R may have the stalk. In this embodiment, the SCR domain-deleted B5R may have the stalk and the transmembrane domain. In this embodiment, the SCR domain-deleted B5R may have the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention include a gene encoding B5R lacking the function of VGF and O1L and having SCR domains 1-4 deleted. In this embodiment, B5R having SCR domains 1-4 deleted may have the stalk. In this embodiment, B5R having SCR domains 1-4 deleted may have the stalk and the transmembrane domain. In this embodiment, B5R having SCR domains 1-4 deleted may have the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention include a gene encoding B5R having the region corresponding to the amino acid sequence shown in SEQ ID NO: 1 deleted. In this embodiment, B5R having the aforementioned region deleted may have the stalk. In this embodiment, B5R having the aforementioned region deleted may have the stalk and the transmembrane domain. In this embodiment, B5R having the aforementioned region deleted may have the stalk, the transmembrane domain and the cytoplasmic tail.

In some embodiments of the invention, in addition to including a polynucleotide encoding IL-7; and a polynucleotide encoding IL-12, the oncolytic vaccinia viruses of the invention lack the function of VGF and O1L, have the SCR domains of B5R deleted, and encode a polypeptide containing the signal peptide, stalk, transmembrane domain and cytoplasmic tail of B5R. In this embodiment, the SCR domain-deleted B5R has the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention lack the function of VGF and O1L, wherein the SCR domain-deleted B5R has an amino acid sequence of B5R corresponding to the amino acid sequence of SEQ ID NO: 2. In this embodiment, the SCR domain-deleted B5R has the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention include a gene encoding B5R lacking the function of VGF and O1L and having SCR domains deleted. In this embodiment, the SCR domain-deleted B5R may have the stalk. In this embodiment, the SCR domain-deleted B5R may have the stalk and the transmembrane domain. In this embodiment, the SCR domain-deleted B5R may have the stalk, the transmembrane domain and the cytoplasmic tail.

In some embodiments of the invention, in addition to including a polynucleotide encoding IL-7; and a polynucleotide encoding IL-12, the oncolytic vaccinia viruses of the invention include a gene encoding B5R lacking the function of VGF and O1L and having SCR domains 1-4 deleted. In this embodiment, B5R having SCR domains 1-4 deleted may have the stalk. In this embodiment, B5R having SCR domains 1-4 deleted may have the stalk and the transmembrane domain. In this embodiment, B5R having SCR domains 1-4 deleted may have the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention include a gene encoding B5R having the region corresponding to the amino acid sequence shown in SEQ ID NO: 1 deleted. In this embodiment, B5R having the aforementioned region deleted may have the stalk. In this embodiment, B5R having the aforementioned region deleted may have the stalk and the transmembrane domain. In this embodiment, B5R having the aforementioned region deleted may have the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention include lack the function of VGF and O1L, have the SCR domains of B5R deleted, and have a gene encoding a polypeptide containing the signal peptide, stalk, transmembrane domain and cytoplasmic tail of B5R. In this embodiment, the SCR domain-deleted B5R has the stalk, the transmembrane domain and the cytoplasmic tail.

In other embodiments of the invention, in addition to including a polynucleotide encoding IL-7; and a polynucleotide encoding IL-12, the oncolytic vaccinia viruses of the invention lack the function of VGF and O1L, wherein the SCR domain-deleted B5R has an amino acid sequence of B5R corresponding to the amino acid sequence of SEQ ID NO: 2. In this embodiment, the SCR domain-deleted B5R has the stalk, the transmembrane domain and the cytoplasmic tail.

The oncolytic vaccinia virus of the invention may be in the intracellular mature virus (IMV) form or in the extracellular enveloped virus (EEV) form. IMV accounts for a large portion of infectious progeny viruses and remains in the cytoplasm of infected cells until the dissolution of the infected cells. When cells are infected in the form of IMV, the form of EEV can be produced in the infected cells. The form of EEV is suitable for remotely infecting cells away from the infected site in the living body and is in the form of covering IMV with a host cell-derived outer membrane (PNAS, 1998, Vol. 95, p. 7544-7549). EEV can be obtained from a vaccinia virus-producing vector or the supernatant of a culture medium of cells infected by the vaccinia virus. A mixture of IMV and EEV can be obtained from a vaccinia virus-producing vector or a cell lysates containing the supernatant of a culture medium of cells infected by the vaccinia virus. The cell lysate can be obtained by an ordinary method (e.g., by destroying cells using an ultrasonic disintegration method or an osmotic shock method). The form of IMV is one of major administration forms for vaccinia viruses.

In one embodiment, the vaccinia virus of the present invention can express the extracellular region of SCR-deleted B5R; however, it is not necessary for the virus to take the EVV form at all times, that is, it is enough if the virus can only express the extracellular region of SCR-deleted B5R on EEV when the EEV form is produced in infected cells.

The vaccinia virus of the present invention can be referred to as a remote infection plasma enhanced-type recombinant vaccinia virus, because it can produce EEV having a higher ability to avoid immunity than a vaccinia virus having a gene encoding wild-type B5R that maintains SCR.

Vaccinia viruses suitable for use in the present invention have oncolytic activity. Examples of methods for evaluating whether or not a test virus has the oncolytic activity include a method for evaluating decrease of the survival rate of cancer cells by the addition of the virus.

Examples of cancer cells to be used for the evaluation include the malignant melanoma cell RPMI-7951 (for example, ATCC HTB-66), the lung adenocarcinoma HCC4006 (for example, ATCC CRL-2871), the lung carcinoma A549 (for example, ATCC CCL-185), the small cell lung cancer cell DMS 53 (for example, ATCC CRL-2062), the lung squamous cell carcinoma NCI-H226 (for example, ATCC CRL-5826), the kidney cancer cell Caki-1 (for example, ATCC HTB-46), the bladder cancer cell 647-V (for example, DSMZ ACC 414), the head and neck cancer cell Detroit 562 (for example, ATCC CCL-138), the breast cancer cell JIMT-1 (for example, DSMZ ACC 589), the breast cancer cell MDA-MB-231 (for example, ATCC HTB-26), the esophageal cancer cell OE33 (for example, ECACC 96070808), the glioblastoma U-87MG (for example, ECACC 89081402), the neuroblastoma GOTO (for example, JCRB JCRB0612), the myeloma RPMI 8226 (for example, ATCC CCL-155), the ovarian cancer cell SK-OV-3 (for example, ATCC HTB-77), the ovarian cancer cell OVMANA (for example, JCRB JCRB1045), the colon cancer cell RKO (for example, ATCC CRL-2577), the colorectal carcinoma HCT 116 (for example, ATCC CCL-247), the pancreatic cancer cell BxPC-3 (for example, ATCC CRL-1687), the prostate cancer cell LNCaP clone FGC (for example, ATCC CRL-1740), the hepatocellular carcinoma JHH-4 (for example, JCRB JCRB0435), the mesothelioma NCI-H28 (for example, ATCC CRL-5820), the cervical cancer cell SiHa (for example, ATCC HTB-35), and the gastric cancer cell Kato III (for example, RIKEN BRC RCB2088).

In one embodiment, suitable vaccinia viruses for use in the present invention do not include a drug-selection marker gene.

Suitable vaccinia viruses for use in the present invention may be expressed and/or proliferated by infecting host cells with the vaccinia virus and culturing the infected host cells. Vaccinia virus may be expressed and/or proliferated by a method known in the field. Host cells to be used to express or proliferate the vaccinia virus according to the present invention, are not particularly limited, as long as the vaccinia virus according to the present invention can be expressed and proliferated. Examples of such host cells include animal cells such as BS-C-1, A549, RK13, HTK-143, Hep-2, MDCK, Vero, HeLa, CV-1, COS, BHK-21, and primary rabbit kidney cells. BS-C-1 (ATCC CCL-26), A549 (ATCC CCL-185), CV-1 (ATCC CCL-70), or RK13 (ATCC CCL-37) may be preferably used. Culture conditions for the host cells, for example, temperature, pH of the medium, and culture time, are selected as appropriate.

Methods for producing the vaccinia virus according to the present invention may include the steps of infecting host cells with the vaccinia virus according to the present invention; culturing the infected host cells; and expressing the vaccinia virus according to the present invention; and optionally collecting and/or purifying the vaccinia virus. Methods that can be used for the purification include DNA digestion with Benzonase, sucrose gradient centrifugation, Iodixanol density gradient centrifugation, ultrafiltration, and diafiltration.

### IV. Methods of Use of the Pharmaceutical Compositions of the Invention

The pharmaceutical compositions of the invention are useful for therapeutic and prophylactic treatment of subjects having a cancer, such as a solid tumor.

As used herein, the terms “treating” or “treatment” refer to a beneficial or desired result including, but not limited to, slowing, alleviation, amelioration, curing, or control of the progression of one or more symptoms associated with cancer. “Treatment” can also mean prolonging survival as compared to expected survival in the absence of treatment. “Treament” encompasses prophylaxis (e.g. preventive measure in a subject at risk of having a cancer. For example, a subject is treated for a cancer if after administration of a pharmaceutical composition, as described herein, the subject shows an observable improvement in clinical status.

The methods of the invention include administering to a subject having a cancer a therapeutically effective amount of a pharmaceutical composition as described herein.

The pharmaceutical composition can be administered by any suitable means known in the art, such as intravenous, intraperitoneal, or intratumoral administration. In certain embodiments, the compositions are administered by intravenous infusion or injection. In other embodiments, the compositions are administered by intratumoral injection.

As used herein, a “therapeutically effective amount” refers to the amount of oncolytic vaccinia virus that is sufficient for producing one or more beneficial results. Such a therapeutically effective amount may vary as a function of various parameters, in particular the mode of administration; the disease state; the age and weight of the subject; the ability of the subject to respond to the treatment; kind of concurrent treatment; the frequency of treatment; and/or the need for prevention or therapy. When prophylactic use is concerned, the pharmaceutical composition of the invention is administered at a dose sufficient to prevent or to delay the onset and/or establishment and/or relapse of a cancer, especially in a subject at risk. For “therapeutic” use, the pharmaceutical composition of the present invention is administered to a subject diagnosed as having a cancer to treat the cancer. In particular, a therapeutically effective amount could be that amount necessary to cause an observable improvement of the clinical status over the baseline status or over the expected status if not treated, e.g. reduction in the tumor number; reduction in the tumor size, reduction in the number or extend of metastasis, increase in the length of remission, stabilization (i.e. not worsening) of the state of disease, delay or slowing of disease progression or severity, amelioration or palliation of the disease state, prolonged survival, better response to the standard treatment, improvement of quality of life, reduced mortality, etc.

A therapeutically effective amount could also be the amount necessary to cause the development of an effective non-specific (innate) and/or specific anti-tumor immune response. Typically, development of an immune response in particular T cell response can be evaluated in vitro, in a biological sample collected from the subject. For example, techniques routinely used in laboratories (e.g. flow cytometry, histology) may be used to perform tumor surveillance. One may also use various available antibodies so as to identify different immune cell populations involved in anti-tumor response that are present in the treated subjects, such as cytotoxic T cells, activated cytotoxic T cells, natural killer cells and activated natural killer cells. An improvement of the clinical status can be easily assessed by any relevant clinical measurement typically used by physicians or other skilled healthcare staff.

In one aspect, the present invention provides a method of treating a subject having a cancer. The methods include administering to the subject, e.g., intratumorally administering to the subject, a therapeutically effective amount of a pharmaceutical composition comprising about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region; and a pharmaceutically acceptable carrier, thereby treating the subject.

In another aspect, the present invention provides a method of treating a subject having a cancer. The methods include administering to the subject, e.g., intratumorally administering to the subject, a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region; tromethamine at a concentration of about 10 mmol/L to about 50 mmol/L; and sucrose at a concentration of about 5% w/v to about 15% w/v, wherein the pH of the composition is about 5.0 to about 8.5, thereby treating the subject.

In certain embodiments of the invention, administration of the pharmaceutical composition to the subject leads to at least one effect selected from the group consisting of inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, antitumor immunity, and stable disease.

In certain embodiments, administration of the pharmaceutical compositions of the invention to a subject induces an abscopal effect.

As used herein, the term “abscopal effect” refers to the ability of a pharmaceutical composition of the invention that is administered locally to a tumor (e.g., intratumoral administration) to shrink untreated tumors concurrently with shrinkage of the tumor that was administered the composition.

Accordingly, in one aspect, the present invention provides a method of treating a subject having a cancer. The method includes administering to the subject, e.g., intratumorally administering to the subject, a therapeutically effective amount of a pharmaceutical composition comprising about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., the hIL12/IL7 virus; and a pharmaceutically acceptable carrier, wherein administration of the pharmaceutical composition to the subject induces an abscopal effect, thereby treating the subject, thereby treating the subject.

In another aspect, the present invention provides a method of inducing an abscopal effect in a subject having a cancer. The methods includes administering to the subject, e.g., intratumorally administering to the subject, a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., the hIL12/IL7 virus; and a pharmaceutically acceptable carrier, thereby inducing an abscopal effect in a subject having a cancer.

In one aspect, the present invention provides a method of treating a subject having a cancer. The methods includes administering to the subject, e.g., intratumorally administering to the subject, a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., the hIL12/IL7 virus; tromethamine at a concentration of about 10 mmol/L to about 50 mmol/L; and sucrose at a concentration of about 5% w/v to about 15% w/v, wherein the pH of the composition is about 5.0 to about 8.5, wherein administration of the pharmaceutical composition to the subject induces an abscopal effect, thereby treating the subject.

In another aspect, the present invention provides a method of inducing an abscopal effect in a subject having a cancer. The methods includes administering to the subject, e.g., intratumorally administering to the subject, a therapeutically effective amount of a pharmaceutical composition comprising, about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region, e.g., the hIL12/IL7 virus; tromethamine at a concentration of about 10 mmol/L to about 50 mmol/L; and sucrose at a concentration of about 5% w/v to about 15% w/v, wherein the pH of the composition is about 5.0 to about 8.5, wherein administration of the pharmaceutical composition to the subject induces an abscopal effect, thereby inducing an abscopal effect in the subject.

The abscopal effect may occur in a metastatic tumor that is proximate to a cancer, such as a tumor, e.g., a primary solid tumor, into which the pharmaceutical composition has been intratumorally administered, or in a metastatic tumor that is remote to a cancer, such as a tumor, e.g., primary solid tumor, into which the pharmaceutical composition has been intratumorally administered.

The present invention also provides a method for inhibiting tumor cell growth in vivo which includes administering, e.g., intratumorally administering, to a subject having a cancer, a therapeutically effective amount of a pharmaceutical composition of the invention. In addition, the present invention provides a method for enhancing an immune response to a cancer cell in a subject having a cancer which includes administering, e.g., intratumorally administering, to a subject having a cancer a therapeutically effective amount of a pharmaceutical composition of the invention.

In one embodiment, the administration of the pharmaceutical compositions of the present invention elicits, stimulates and/or re-orients an immune response. In particular, the administration induces a protective T or B cell response in the treated host, e.g., against the oncolytic virus. The protective T cell response can be CD4+ or CD8+ or both CD4+ and CD8+cell mediated. B cell response can be measured by ELISA and T cell response can be evaluated by conventional ELISpot, ICS assays from any sample (e.g., blood, organs, tumors, etc) collected from the subject.

The dose of a pharmaceutical composition administered to a subject, e.g., intratumorally administered to a subject, may be about 1×10⁶ to about 1×10¹⁰, about 1×10⁷ to about 1×10⁹, about 1×10⁷, 5×10⁷, about 1×10⁸, about 5×10⁸, about 1×10⁸, or about 5×10⁸ pfu. Ranges and values intermediate to the above recited ranges and values are also intended to be part of this invention. In addition, ranges of values using a combination of any of the above recited values as upper and/or lower limits are intended to be included.

The volume of a dose of a pharmaceutical composition of the invention comprising, e.g., about 5.0×10⁸ pfu/ml of the oncolytic vaccinia virus, suitable for administering, e.g., intratumorally administering, to the subject may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or about 6.0 ml. Ranges and values intermediate to the above recited ranges and values are also intended to be part of this invention. In addition, ranges of values using a combination of any of the above recited values as upper and/or lower limits are intended to be included.

In certain embodiments, the dose of the pharmaceutical composition administered to the subject, e.g., intratumorally, is in a volume that achieves an injection ratio of about 0.2 to about 0.8 (volume of pharmaceutical composition/tumor volume), e.g., about 0.2 to about 0.6, about 0.4 to about 0.8, about 0.4 to about 0.6, or about 0.6 to about 0.8.

The pharmaceutical compositions of the invention may administered to the subject once every week, once every two weeks, once every three weeks, or once every four weeks. In one embodiment, the pharmaceutical composition of the invention is administered to the subject once every two weeks.

The pharmaceutical compositions of the invention may be administered to the subject once or more than once.

In some embodiments, the pharmaceutical compositions of the invention are administered to the subject in a dosing regimen. For example, in one embodiment, a suitable dosing regimen may include administering to the subject a first dose of the pharmaceutical composition on day 1 and a second dose of the pharmaceutical composition on day 15. The dosing regimen may be administered to the subject once or may be repeated. For example, in one embodiment, a dosing regimen of the invention which includes administering to the subject a first dose of the pharmaceutical composition on day 1 and a second dose of the pharmaceutical composition on day 15 is repeated beginning at day 28 following the first dose of the pharmaceutical composition.

Subjects, such as human subjects, that would benefit from treatment with the pharmaceutical compositions of the invention include subjects having a cancer. The cancer may be a primary tumor, such as a solid tumor, e.g., an advanced solid tumor, or a metastatic tumor.

The cancer may a malignant melanoma, lung adenocarcinoma, lung cancer, small cell lung cancer, lung squamous carcinoma, kidney cancer, bladder cancer, head and neck cancer, breast cancer, esophageal cancer, glioblastoma, neuroblastoma, myeloma, ovarian cancer, colorectal cancer, pancreatic cancer, prostate cancer, hepatocellular carcinoma, mesothelioma, cervical cancer or gastric cancer.

In some embodiments, the cancer is a cutaneous, subcutaneous, mucosal or submucosal tumor.

In other embodiments, the cancer is a primary or metastatic solid tumor in a location other than a cutaneous, a subcutaneous, a mucosal or a submucosal location.

In yet other embodiments, the cancer is a head and neck squamous cell carcinoma, a dermatological cancer, a nasopharyngeal cancer, a sarcoma, or a genitourinary/gynecological tumor.

In one embodiment, the cancer is a primary or metastatic tumor of the liver. In another embodiment, the cancer may be a primary or metastatic gastric tumor.

Suitable subjects that would benefit from the methods of the invention, such as human subjects, may be adult subjects, e.g., subjects that are about 18 years of age or older; adolescent subjects, e.g., subjects that are between about 10 and 18 years of age; or pediatric subjects, e.g., subjects under the age of 18.

The methods of the invention may be practiced alone or in combination with additional therapeutic agents or therapies, such as surgery, radiation, chemotherapy, immunotherapy, hormone therapy.

The additional therapeutic agent or therapy may be administered to the subject before, after or concurrently with administration of a pharmaceutical composition of the invention.

The additional therapeutic agent may be present in the same pharmaceutical compositions as the pharmaceutical composition comprising an oncolytic vaccinia virus of the invention, or the additional therapeutic agent may be present in a pharmaceutical composition separate from the pharmaceutical composition comprising an oncolytic vaccinia virus of the invention.

In some embodiments, the additional therapeutic agent is an alkylating agent such as mitomycin C, cyclophosphamide, busulfan, ifosfamide, isosfamide, melphalan, hexamethylmelamine, thiotepa, chlorambucil, or dacarbazine.

In some embodiments, the additional therapeutic agent is an antimetabolite, such as, gemcitabine, capecitabine, 5-fluorouracil, cytarabine, 2-fluorodeoxy cytidine, methotrexate, idatrexate, tomudex or trimetrexate.

In some embodiments, the additional therapeutic agent is a topoisomerase II inhibitor such as, doxorubicin, epirubicin, etoposide, teniposide or mitoxantrone;

In some embodiments, the additional therapeutic agent is a topoisomerase I inhibitor such as, irinotecan (CPT-11), 7-ethyl-10-hydroxy-camptothecin (SN-38) or topotecan.

In some embodiments, the additional therapeutic agent is an antimitotic drug, such as, paclitaxel, docetaxel, vinblastine, vincristine or vinorelbine.

In some embodiments, the additional therapeutic agent is a platinum derivative such as, e.g., cisplatin, oxaliplatin, spiroplatinum or carboplatinum.

In some embodiments, the additional therapeutic agent is an inhibitor of tyrosine kinase receptors such as sunitinib (Pfizer) and sorafenib (Bayer).

In some embodiments, the additional therapeutic agent is an anti-neoplastic antibody in particular antibodies that affect the regulation of cell surface receptors such as trastuzumab, cetuximab, panitumumab, zalutumumab, nimotuzumab, matuzumab, bevacizumab and ranibizumab.

In some embodiments, the additional therapeutic agent is an EGFR (for Epidermal Growth Factor Receptor) inhibitor such as gefitinib, erlotinib and lapatinib.

In some embodiments, the additional therapeutic agent is an immunomodulatory agent such as, e.g., alpha, beta or gamma interferon, interleukin (in particular IL-2, IL-6, IL-10 or IL-12) or tumor necrosis factor.

In other embodiments of the invention, the methods may include the administration of additional therapeutic agents, such as a cancer vaccine, a checkpoint inhibitor, a lymphocyte activation gene 3 (LAG3) inhibitor, a glucocorticoid-induced tumor necrosis factor receptor (GITR) inhibitor, a T-cell immunoglobulin and mucin-domain containing-3 (TIM3) inhibitor, a B- and T-lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a bispecific anti-CD3/anti-CD20 antibody, a vascular endothelial growth factor (VEGF) antagonist, an angiopoietin-2 (Ang2) inhibitor, a transforming growth factor beta (TGFβ) inhibitor, a CD38 inhibitor, an epidermal growth factor receptor (EGFR) inhibitor, granulocyte-macrophage colony stimulating factor (GM-CSF), cyclophosphamide, an antibody to a tumor-specific antigen, Bacillus Calmette-Guerin vaccine, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and/or a dietary supplement.

In certain embodiments, the additional therapeutic agent is a checkpoint inhibitor. Accordingly, the methods of the invention further include administering to the subject a therapeutically effective amount of a checkpoint inhibitor.

The terms “checkpoint inhibitor” or “immune checkpoint inhibitor,” as used herein, refer to a molecule capable of inhibiting the function of a checkpoint protein, such as the interaction between an antigen presenting cell (APC) or a cancer cell and a T effector cell. The term “immune checkpoint” refers to a protein directly or indirectly involved in an immune pathway that under normal physiological conditions is crucial for preventing uncontrolled immune reactions and, thus, for the maintenance of self-tolerance and/or tissue protection.

Suitable checkpoint inhibitors include a programmed cell death 1 (PD-1) inhibitor; a programmed cell death ligand 1 (PD-L1) inhibitor; a cytotoxic T lymphocyte associated protein 4 (CTLA-4) inhibitor; a T-cell immunoglobulin domain and mucin domain-3 (TIM-3) inhibitor; a lymphocyte activation gene 3 (LAG-3) inhibitor; a T cell immunoreceptor with Ig and ITIM domains (TIGIT) inhibitor; a B and T lymphocyte associated (BTLA) inhibitor; or a V-type immunoglobulin domain-containing suppressor of T-cell activation (VISTA) inhibitor. The immune checkpoint inhibitor can bind to an immune checkpoint molecule or a ligand thereof, for example, to inhibit immune suppression signals, thereby inhibiting the immune checkpoint function. By way of example, it can inhibit binding between PD-1 and PD-L1 or PD-L2 to thereby inhibit PD-1 signals. Or, it can inhibit binding between CTLA-4 and CD80 or CD86 to thereby inhibit CTLA-4 signals (Matthieu Collin, Expert Opinion on Therapeutic Patents, 2016, Vol. 26, p. 555-564).

PD-1 is a protein referred to as programmed cell death-1 and is also called PDCD-1 or CD279. PD-1 is a membrane protein of immunoglobulin super family, plays a role of suppressing the activation of T cells by binding PD-L1 or PD-L2, and is believed to be contributing to the prevention of autoimmune diseases. Cancer cells express PD-L1 on the surface thereof in order to control T cells negatively and thereby avoiding attacks from T cells. PD-1 includes human PD-1 (e.g., PD-1 having an amino acid sequence registered in Accession No. NP_005009.1 of Genbank). PD-1 includes PD-1 having an amino acid sequence corresponding to the amino acid sequence registered in Accession No. NP_005009.1. As used herein, the term “amino acid sequence corresponding to” is used to include functional PD-1 in which orthologs and naturally occurring amino acid sequences are not completely identical.

PD-L1 is a ligand of PD-1 and is also referred to as B7-H1 or CD274. PD-L1 includes human PD-L1, for example (e.g., PD-L1 having an amino acid sequence registered in Accession No. NP_054862.1 of Genbank). PD-1 includes PD-1 having an amino acid sequence corresponding to the amino acid sequence registered in Accession No. NP_054862.1.

PD-L2 is a ligand of PD-1 and is also referred to as B7-DC or CD273. PD-L2 includes human PD-L2, for example (e.g., PD-L2 having an amino acid sequence registered in Accession No. AAI13681.1 of Genbank). PD-2 includes PD-2 having an amino acid sequence corresponding to the amino acid sequence registered in Accession No. AAI13681.1 of Genbank.

CTLA-4 is a membrane protein of immunoglobulin super family and is expressed in activated T cells. CTLA-4 is similar to CD28 and is bound to CD80 and CD86 on antigen-presenting cells. It is known that CTLA-4 sends inhibitory signals to T cells, while CD28 sends co-stimulatory signals to T cells. CTLA-4 includes human CTLA-4, for example (e.g., CTLA-4 having an amino acid sequence registered in Accession No. AAH74893.1 of Genbank). CTLA-4 includes CTLA-4 having an amino acid sequence corresponding to the amino acid sequence registered in Accession No. AAH74893.1 of Genbank.

CD80 and CD86 are membrane proteins of immunoglobulin super family, are expressed in a wide variety of hematopoietic cells and interact with CD28 and CTLA-4 on the surface of T cells as described above. CD80 includes human CD80 (e.g., CD80 having an amino acid sequence registered in Accession No. NP_005182.1 of Genbank). CD80 includes CD80 having an amino acid sequence corresponding to the amino acid sequence registered in Accession No. NP_005182.1 of Genbank. CD86 includes human CD86 (e.g., CD86 having an amino acid sequence registered in Accession No. NP_787058.4 of Genbank). CD86 includes CD86 having an amino acid sequence corresponding to the amino acid sequence registered in Accession No. NP_787058.4 of Genbank.

In certain embodiments of the invention, a suitable immune checkpoint inhibitor is a checkpoint inhibitor that blocks signals sent via PD-1 or a checkpoint inhibitor that blocks signals sent via CTLA-4. The immune checkpoint inhibitor may be an antibody capable of neutralizing binding between PD-1 and PD-L1 or PD-L2, and an antibody capable of neutralizing binding between CTLA-4 and CD80 or CD86. The antibody that can neutralize binding between PD-1 and PD-L1 includes an anti-PD-1 antibody that can neutralize binding between PD-1 and PD-L1 and an anti-PD-L1 antibody that can neutralize binding between PD-1 and PD-L1. The antibody that can neutralize binding between PD-1 and PD-L2 includes anti-PD-1 and anti-PD-L2 antibodies that can neutralize binding between PD-1 and PD-L2. The antibody that can neutralize binding between CTLA-4 and CD80 or CD86 includes an anti-CTLA-4 antibody that can neutralize binding between CTLA-4 and CD80 or CD86.

An antibody capable of neutralizing the binding of two proteins can be obtained by first finding antibodies that can bind to either one of those two proteins and then sorting the obtained antibodies out on the basis of the ability of neutralizing the binding of those two proteins.

By way of example, the antibody capable of neutralizing binding between PD-1 and PD-L1 can be obtained by finding antibodies that can bind to either PD-1 or PD-L1 and then sorting the obtained antibodies out on the basis of the ability of neutralizing binding between PD-1 and PD-L1. Moreover, for example, the antibody capable of neutralizing binding between PD-1 and PD-L2 can be obtained by finding antibodies that can bind to either PD-1 or PD-L2 and then sorting the obtained antibodies out on the basis of the ability of neutralizing binding between PD-1 and PD-L2. Moreover, for example, the antibody capable of neutralizing binding between CTLA-4 and CD80 or CD86 can be obtained by finding antibodies that can bind to CTLA-4 and then sorting the obtained antibodies out on the basis of the ability of neutralizing binding between CTLA-4 and CD80 or CD86.

An antibody binding to a certain protein can be obtained using a method well known to those skilled in the art. The ability of an antibody to neutralize the binding of two proteins may be examined by immobilizing one protein, adding the other protein from a liquid phase and then examining whether or not the antibody can lower the binding amount thereof. For example, a protein to be added from the liquid phase is labelled, and it can be decided that the antibody can neutralize the binding of those two proteins if the amount of labels declines by adding the antibody.

As used herein, the term “antibody” refers to an immunoglobulin, and more particularly to a biological molecule containing two heavy chains (H chains) and two light chains (L chains), which are stabilized with disulfide bonds. The heavy chain consists of heavy variable regions (VH), heavy constant regions (CH1, CH2, CH3), and a hinge region disposed between CH1 and CH2, and the light chain consists of light variable regions (VL) and light constant regions (CL). Among these, the variable region fragment (Fv) consisting of VH and VL is directly involved in antigen binding, thereby giving diversity to an antibody. A region consisting of the hinge region, CH2 and CH3 is referred to as the Fc region.

In the variable region, the region directly coming into contact with an antigen is altered particularly significantly and referred to as the complementarity-determining region (CDR). The portion other than CDR that has less mutation is referred to as the framework region. Three CDRs exist in the variable region between the light chain and the heavy chain and are referred to as heavy chains CDRs 1-3 and light chains CDRs 1-3 sequentially from the N-terminal side.

The antibody may be a monoclonal antibody or a polyclonal antibody; however, a monoclonal antibody is preferably used in the present invention. The antibody may be any one of isotypes, i.e., IgG, IgM, IgA, IgD, and IgE. The antibody may be prepared by immunizing non-human animals such as mice, rats, hamsters, guinea pigs, rabbits, and chickens, and may be a recombinant antibody, a chimeric antibody, a humanized antibody, a human antibody and what not. The chimeric antibody refers to an antibody prepared by linking fragments derived from different species, and the humanized antibody refers to an antibody prepared by replacing CDRs of an antibody of a non-human animal (e.g., a non-human mammal) with the corresponding complementarity-determining regions of a human antibody. The humanized antibody may be an antibody in which CDRs are derived from a non-human animal and the other portions are derived from a human. The human antibody is also referred to as a fully human antibody and is an antibody in which all portions of an antibody are constituted of amino acid sequences encoded by human antibody genes. In the present invention, a chimeric antibody may be used according to one embodiment, a humanized antibody according to another embodiment, and a human antibody (fully human antibody) according to another embodiment.

As used herein, the term “antigen-binding fragment” refers to a fragment of an antibody that can bind to an antigen. More specifically, the antigen-binding fragment includes Fab consisting of VL, VH, CL and CH1 regions, F(ab′) 2 in which two Fabs are linked together with disulfide bonds, bispecific antibodies such as Fv consisting of VL and VH, scFv which is a single-chain antibody prepared by linking VL and VH with an artificially-made polypeptide linker, diabodies, single-chain diabody (scDb) types, tandem scFv types and leucine zipper types, and heavy chain antibodies such as VHH antibodies (Ulrich Brinkmann et al., MAbs, 2017, Vol. 9, No. 2, p. 182-212).

An immune checkpoint inhibitor that can be used in the present invention may also include an antigen-binding fragment that suppresses immune suppression signals by binding to an immune checkpoint molecule or a ligand thereof, a vector that expresses an antigen-binding fragment in the living body, and an immune checkpoint inhibitor containing a low molecular weight compound.

In one embodiment, an immune checkpoint inhibitor for use in the present invention is an antibody selected from the group consisting of an anti-PD-1 antibody, or antigen-binding fragment thereof; an anti-PD-L1 antibody, or antigen-binding fragment thereof; an anti-CTLA-4 antibody, or antigen-binding fragment thereof; an anti-TIM-3 antibody, or antigen-binding fragment thereof; an anti-LAG-3 antibody, or antigen-binding fragment thereof; an anti-TIGIT antibody, or antigen-binding fragment thereof; and anti-BTLA antibody, or antigen-binding fragment thereof; and anti-VISTA antibody, or antigen-binding fragment thereof; such as JNJ-61610588 (International Publication No. 2016/207717). Suitable anti-immune checkpoint antibodies, or antigen binding fragments thereof, may be human antibodies, chimeric antibodies, or humanized antibodies.

In another embodiment, an immune checkpoint inhibitor for use in the present invention is an antibody selected from the group consisting of an anti-PD-1 antibody, or antigen-binding fragment thereof, an anti-PD-L1 antibody, or antigen-binding fragment thereof; and an anti-CTLA-4 antibody, or antigen-binding fragment thereof. Suitable anti-immune checkpoint antibodies, or antigen binding fragments thereof, may be human antibodies, chimeric antibodies, or humanized antibodies.

An anti-immune checkpoint antibody, or antigen binding fragment thereof, may be administered to the subject before, after or concurrently with administration of a pharmaceutical composition of the invention. In one embodiment, an anti-immune checkpoint antibody, or antigen binding fragment thereof, is administered to the subject after administration of a pharmaceutical composition of the invention. In another embodiment, an anti-immune checkpoint antibody, or antigen binding fragment thereof, is administered to the subject before administration of a pharmaceutical composition of the invention.

In certain aspects, a pharmaceutical composition comprising an oncolytic vaccinia virus of the invention and an immune checkpoint inhibitor are administered to a subject having a cancer in accordance with an administration schedule including an administration cycle.

For example, in one embodiment, in one or more cycles of an administration schedule, a pharmaceutical composition comprising an oncolytic vaccinia virus of the invention may first be administered to subject having a cancer and subsequently an immune checkpoint inhibitor, such as an anti-immune checkpoint antibody, or antigen binding fragment thereof, is administered to the subject

In another embodiment, one or more cycles of an administration schedule in which a pharmaceutical composition comprising an oncolytic vaccinia virus of the invention is to be first administered to a subject having a cancer may be completed and then an immune checkpoint inhibitor, such as an anti-immune checkpoint antibody, or antigen binding fragment thereof, is administered to the subject

In one embodiment, in one or more cycles of an administration schedule, an immune checkpoint inhibitor, such as an anti-immune checkpoint antibody, or antigen binding fragment thereof, may first be administered to subject having a cancer and subsequently a pharmaceutical composition comprising an oncolytic vaccinia virus of the invention, is administered to the subject

In another embodiment, in one or more cycles of an administration schedule in which an immune checkpoint inhibitor, such as an anti-immune checkpoint antibody, or antigen binding fragment thereof, is to be first administered to a subject having a cancer may be completed and then a pharmaceutical composition comprising an oncolytic vaccinia virus of the invention is administered to the subject

As indicated above, in certain embodiments, an immune checkpoint inhibitor is an anti-immune checkpoint inhibitor antibody, such as, an anti-PD-1 antibody, such as Nivolumab, Pembrolizumab and Pidilizumab; and anti-PD-L1 antibody, such as Atezolizumab, Durvalumab and Avelumab; an anti-CTLA-4 antibody, such as Ipilimumab; an anti-TIM-3 antibody, such as TSR-022 (International Publication No. 2016/161270) and MBG453 (International Publication No. 2015/117002); an anti-LAG-3 antibody, such as LAG525 (International Publication No. 2015/0259420), an anti-TIGIT antibody, such as MAB10 (International Publication No. 2017/059095); and anti-BTLA antibody, such as BTLA-8.2 (J. Clin. Investig. 2010; 120:157-167), and anti-VISTA antibodies such as JNJ-61610588 (International Publication No. 2016/207717).

The invention is further illustrated by the following examples, which should not be construed as further limiting. The contents of all references, pending patent applications and published patents, cited throughout this application are hereby expressly incorporated by reference.

### EXAMPLES

In the following examples, the vaccinia virus used to conduct the studies using tumor-bearing immunodeficient mice and non-human primates is an attenuated recombinant vaccinia virus expressing human transgenes for interleukin-12 (IL-12) and interleukin-7 (IL-7) that was designed to replicate selectively in cancer cells and is interchangeably referred to herein as “LC16mO ΔSCR VGF-SP-IL12/O1L-SP-IL7,” “the hIL12 and hIL7-carrying vaccinia virus,” and “the hIL12/hIL7 virus.” A schematic of the hIL12 and hIL7-carrying vaccinia virus viral genome is depicted in FIG. 19.” In the hIL12 and hIL7-carrying vaccinia virus, the virulence genes for virus growth factor (VGF) and O1L have been functionally inactivated by insertion of the genes expressing human IL-12 and human IL-7 into these 2 loci, respectively. In addition, the B5R membrane protein has been modified for reduced antigenicity by deleting SCR domains 1-4.

To test the antitumor immune response of the hIL12 and hIL7-carrying vaccinia virus in immunocompetent mice, a surrogate of the hIL12 and hIL7-carrying vaccinia virus virus (“the hIL12 and hIL7-carrying vaccinia virus-surrogate”) carrying transgenes that express murine interleukin-12 (IL-12) and human interleukin-7 (IL-7) was prepared because of the lack of cross-reactivity of human IL-12 in the mouse (Schoenhaut et al, J Immunol. 1992; 148:3433-40). The structure of the hIL12 and hIL7-carrying vaccinia virus-surrogate is same as that of the hIL12 and hIL7-carrying vaccinia virus with the exception that the gene for murine IL-12 was inserted into the virus growth factor (VGF) locus instead of that of human IL-12.

The pharmaceutical formulation used in most of the non-clinical studies described below was the hIL12 and hIL7-carrying vaccinia virus or the hIL12 and hIL7-carrying vaccinia virus-surrogate suspended in 30 mmol/L Tris-HCl containing 10% sucrose and purified with tangential flow filtration. This purification method will also be used to obtain drug substance. In other nonclinical studies, the hIL12 and hIL7-carrying vaccinia virus or the hIL12 and hIL7-carrying vaccinia virus-surrogate was concentrated by density gradient ultracentrifugation.

### Example 1. Cytotoxic Effect of the hIL12 and hIL7-Carrying Vaccinia Virus in Human Tumor Cells

This study was conducted to determine whether the hIL12 and hIL7-carrying vaccinia virus shows a cytotoxic effect in the following human cancer cell lines: human colorectal carcinoma (COLO 741) cells, human glioblastoma (U-87 MG) cells and human cholangiocarcinoma (HuCCT1) cells.

All cells were infected with the hIL12 and hIL7-carrying vaccinia virus at various multiplicities of infection (MOIs) (0, 0.1, 1, 10 and 100). At 45 days post-infection, cell viability was measured using the CellTiter-Glo®2.0 Assay. Cell viability was calculated by setting uninfected cells (MOI 0) and medium control wells containing no cells to 100% and 0% survival, respectively. One experiment was performed, and the data were expressed as the mean of triplicate measures.

At 4 days after infection, the cell viabilities were decreased to <10% (Table 1). These results indicate the hIL12 and hIL7-carrying vaccinia virus is cytotoxic against COLO 741, U-87 MG and HuCCT1 cells.

TABLE 1
Cytotoxic Effect of the hIL12 and hIL7-Carrying Vaccinia Virus

| The hIL12 and hIL7- | | | | | | |
|---|---|---|---|---|---|---|
| carrying vaccinia | % Cell Viability mean (±SEM) | | | | | |
| virus MOI | COLO 741 | | U-87 MG | | HuCCT1 | |
| 100 | 6.4 | (0.7) | 6.3 | (0.1) | 0.8 | (0.0) |
| 10 | 23.2 | (1.4) | 16.5 | (0.7) | 19.4 | (1.2) |
| 1 | 72.0 | (1.0) | 66.2 | (2.4) | 89.3 | (0.5) |
| 0.1 | 100.4 | (0.5) | 97.5 | (0.9) | 101.2 | (0.2) |

COLO 741: human colorectal carcinoma cell line; HuCCT1: human cholangiocarcinoma cell line; MOI: multiplicity of infection; U-87 MG: human glioblastoma cell line.

### Example 2. Cytotoxic Activity of the hIL12 and hIL7-Carrying Vaccinia Virus Against Various Human Cancer Cell Lines

This study was conducted to further examine whether the hIL12 and hIL7-carrying vaccinia virus shows a cytotoxic effect against 24 human cancer cell lines.

Cells were infected with the hIL12 and hIL7-carrying vaccinia virus at various multiplicity of infection (MOIs). At 5 days postinfection, cell viability was measured using the CellTiter-Glo® Luminescent Cell Viability Assay. Cell viability was calculated by setting uninfected cells (MOI 0) and medium control wells containing no cells to 100% and 0% survival, respectively. One experiment was performed, and the data were expressed as the mean of triplicate measures.

As depicted in FIG. 1, the hIL12 and hIL7-carrying vaccinia virus is cytotoxic against all examined human cancer cells at 5 days after the infection at an MOI of 1.0, 10 or 100.

### Example 3. Replication of the hIL12 and hIL7-Carrying Vaccinia Virus in Human

Cancer Cells or Normal Cells

This study was conducted to examine whether the hIL12 and hIL7-carrying vaccinia virus selectively replicates in human cancer cells over normal cells.

Human cancer cells (NCI-H520, HARA, LK-2 and LUDLU 1) or normal human bronchial epithelial cells (HBEpC) were infected with the hIL12 and hIL7-carrying vaccinia virus at an MOI of 1 or vehicle (MOI 0). Cells were harvested at 6 hours or 24 hours after the infection and the amount of DNA of the hIL12 and hIL7-carrying vaccinia virus was measured by standard quantitative polymerase chain reaction (qPCR) with primers designed to amplify the vaccinia virus hemagglutinin (HA) J7R gene. Values were normalized to the 18s ribosomal RNA gene and expressed as the mean of duplicate measures.

As depicted in FIG. 2, higher amounts of genomic DNA of the hIL12 and hIL7-carrying vaccinia virus were detected in all of the human cancer cells than in normal cells, HBEpC, at 24 hours after the infection with the hIL12 and hIL7-carrying vaccinia virus, although no obvious difference was observed among all tested cells at 6 hours after the infection.

This result demonstrates that the hIL12 and hIL7-carrying vaccinia virus replicates more selectively in human cancer cells than in normal cells.

### Example 4. Secretion of Transgene Products from Human Tumor Cells Infected with the hIL12 and hIL7-Carrying Vaccinia Virus

This study was conducted to examine whether transgene products are secreted from human cancer cells, COLO 741, U-87 MG. and HuCCT1, after infection with the hIL12 and hIL7-carrying vaccinia virus.

All cancer cells were infected with the hIL12 and hIL7-carrying vaccinia virus at an MOI of 0 or 1 and cultured for 2 days. The cell culture supernatants were then collected and secreted human IL-12 protein was detected using the Human IL-12 p70 DuoSet® ELISA or secreted human IL-7 protein was detected using the Human IL-7 ELISA kit. Three independent experiments were performed in triplicate, and data are shown as mean (±SEM) of the 3 experiments.

As shown in Tables 2 and 3, human IL-12 and human IL-7 proteins were detected in all the culture supernatants of cells infected with the hIL12 and hIL7-carrying vaccinia virus at an MOI of 1, but not detected in the culture supernatants of uninfected cells.

In conclusion, secretion of the transgene products was confirmed in the cell culture supernatants of all the tested cell lines infected with the hIL12 and hIL7-carrying vaccinia virus.

TABLE 2
Amount of Secreted Human IL-12 Protein

The hIL12 and

| hIL7-carrying | Human IL-12 mean ± SEM (ng/mL) | | |
|---|---|---|---|
| vaccinia virus MOI | COLO 741 | U-87 MG | HuCCT1 |
| MOI 1 | 6.9 (0.1) | 11.5 (0.5) | 8.9 (0.6) |
| MOI 0 | not detected | not detected | not detected |

COLO 741: human colorectal carcinoma cell line; ELISA: enzyme-linked immunosorbent assay; HuCCT1: human cholangiocarcinoma cell line; IL-12: interleukin-12; MOI: multiplicity of infection; not detected: less than the limit of quantification (<0.3125 ng/mL) of the ELISA kit used; U-87 MG: human glioblastoma cell line.

TABLE 3
Amount of Secreted Human IL-7 Protein

The hIL12 and

| hIL7-carrying | Human IL-7 mean ± SEM (ng/mL) | | |
|---|---|---|---|
| vaccinia virus MOI | COLO 741 | U-87 MG | HuCCT1 |
| MOI 1 | 71.1 (0.2) | 37.2 (3.0) | 31.5 (1.7) |
| MOI 0 | not detected | not detected | not detected |

COLO 741: human colorectal carcinoma cell line; ELISA: enzyme-linked immunosorbent assay; HuCCT1: human cholangiocarcinoma cell line; IL-7: interleukin-7; MOI: multiplicity of infection; not detected: less than the limit of quantification (<0.04115 ng/mL) of the ELISA kit used; U-87 MG: human glioblastoma cell line.

### Example 5. Cytotoxic Effect of the hIL12 and hIL7-Carrying Vaccinia Virus in Human Tumor Cells

This study was conducted to examine whether the hIL12 and hIL7-carrying vaccinia virus-surrogate carrying murine IL-12 and human IL-7 shows cytotoxic effects in human cancer cell lines COLO 741, U-87 MG and HuCCT1.

All cancer cell lines were infected with the hIL12 and hIL7-carrying vaccinia virus-surrogate at various MOIs ranging from 0 to 100. At 4 days postinfection, cell viability was measured using the CellTiter-Glo® Luminescent Cell Viability Assay. Cell viability was calculated by setting uninfected cells (MOI 0) and medium control wells containing no cells to 100% and 0% survival, respectively. Three independent experiments were performed in triplicate wells, and data are shown as mean (±SEM) of the 3 experiments.

As shown in Table 4, at 4 days after the infection, cell viabilities were decreased to <20% at MOIs of 11, 33 and 100.

Accordingly, the hIL12 and hIL7-carrying vaccinia virus-surrogate showed cytotoxic effects against human cancer cells (COLO 741, U-87 MG and HuCCT1 cells) similar to the hIL12 and hIL7-carrying vaccinia virus.

TABLE 4
Cytotoxic Effect of the hIL12 and hIL7-Carrying Vaccinia
Virus -surrogate Against Human Cancer Cell Lines

| The hIL12 and hIL7- | | | | | | |
|---|---|---|---|---|---|---|
| carrying vaccinia virus- | % Cell Viability mean (±SEM) | | | | | |
| surrogate (MOI) | COLO 741 | | U-87 MG | | HuCCT1 | |
| 0 | 100.0 | (0.0) | 100.0 | (0.0) | 100.0 | (0.0) |
| 0.02 | 101.5 | (0.6) | 101.4 | (0.4) | 101.2 | (0.7) |
| 0.05 | 100.8 | (0.8) | 100.3 | (0.5) | 102.0 | (1.4) |
| 0.1 | 99.8 | (0.5) | 98.3 | (1.7) | 103.4 | (0.8) |
| 0.4 | 93.5 | (1.4) | 84.7 | (4.5) | 98.9 | (1.0) |
| 1.2 | 66.4 | (2.1) | 49.6 | (6.7) | 90.3 | (0.6) |
| 3.7 | 31.9 | (0.4) | 22.0 | (2.9) | 61.6 | (0.4) |
| 11 | 16.3 | (0.6) | 11.2 | (0.4) | 16.3 | (0.3) |
| 33 | 7.6 | (0.2) | 8.5 | (0.8) | 1.9 | (0.1) |
| 100 | 3.0 | (0.1) | 6.2 | (0.2) | 0.7 | (0.0) |

COLO 741: human colorectal carcinoma cell line; HuCCT1: human cholangiocarcinoma cell line; MOI: multiplicity of infection; U-87 MG: human glioblastoma cell line.

### Example 6. Secretion of Transgene Products from Human Tumor Cells Infected with the hIL12 and hIL7-Carrying Vaccinia Virus-Surrogate

This study was conducted to examine whether transgene products are secreted from human cancer cells COLO 741, U-87 MG and HuCCT1, after infection with the hIL12 and hIL7-carrying vaccinia virus-surrogate.

All cancer cells were infected with the hIL12 and hIL7-carrying vaccinia virus-surrogate at an MOI of 0 or 1 and cultured for 2 days. At 2 days postinfection, cell culture supernatants were collected and secreted murine IL-12 protein was detected using the Murine IL-12 p70 DuoSet® ELISA or secreted human IL-7 protein was detected using the Human IL-7 ELISA kit. Three independent experiments were performed in triplicate, and data are shown as mean (+SEM) of the 3 experiments.

As shown in Tables 5 and 6, murine IL-12 and human IL-7 proteins were detected in all the culture supernatants of cells infected with the hIL12 and hIL7-carrying vaccinia virus-surrogate but not detected in the culture supernatants of uninfected cells.

In conclusion, secretion of the transgene products was confirmed in the cell culture supernatants of all tested cell lines infected with the hIL12 and hIL7-carrying vaccinia virus-surrogate.

TABLE 5
Amount of Secreted Murine IL-12 Protein

The hIL12 and hIL7-

| carrying vaccinia virus- | Murine IL-12 mean ± SEM (ng/mL) | | |
|---|---|---|---|
| surrogate MOI | COLO 741 | U-87 MG | HuCCT1 |
| MOI 1 | 86.2 (8.2) | 392.4 (8.5) | 89.8 (5.1) |
| MOI 0 | not detected | not detected | not detected |

COLO 741: human colorectal carcinoma cell line; ELISA: enzyme-linked immunosorbent assay; HuCCT1: human cholangiocarcinoma cell line; IL-12: interleukin-12; MOI: multiplicity of infection; not detected: less than the limit of quantification of the ELISA kit used; U-87 MG: human glioblastoma cell line.

TABLE 6
Amount of Secreted Human IL-7 Protein

The hIL12 and hIL7-

| carrying vaccinia virus- | Human IL-7 mean ± SEM (ng/mL) | | |
|---|---|---|---|
| surrogate MOI | COLO 741 | U-87 MG | HuCCT1 |
| MOI 1 | 55.4 (10.1) | 114.5 (11.8) | 27.5 (2.2) |
| MOI 0 | not detected | not detected | not detected |

COLO 741: human colorectal carcinoma cell line; ELISA: enzyme-linked immunosorbent assay; HuCCT1: human cholangiocarcinoma cell line; IL-7: interleukin-7; MOI: multiplicity of infection; not detected: less than the limit of quantification of the ELISA kit used; U-87 MG: human glioblastoma cell line.

### Example 7. Antitumor Activity of Intratumoral Administration of the hIL12 and hIL7-Carrying Vaccinia Virus in Immunocompromised Mice Subcutaneously Xenografted with Human Colorectal Carcinoma Cells or Glioblastoma Cells

This study was conducted to investigate the antitumor effect of the hIL12 and hIL7-carrying vaccinia virus in nude mice subcutaneously inoculated with COLO 741 or U-87 MG. After establishment of the tumor, the hIL12 and hIL7-carrying vaccinia virus at a dose range of 2×10³ to 2×10⁷ pfu/mouse was intratumorally injected in tumor-bearing mice on day 1. Statistical analysis was performed for the values on day 21.

In the COLO 741 xenograft model, the hIL12 and hIL7-carrying vaccinia virus significantly inhibited the tumor growth at doses ≥2×10⁵ pfu/mouse and induced tumor regression at 2×10⁷ pfu/mouse on day 21 (FIG. 3A) In this model, the hIL12 and hIL7-carrying vaccinia virus did not induce body weight loss compared to the control group (FIG. 3B). In the U-87 MG xenograft model, the hIL12 and hIL7-carrying vaccinia virus also significantly inhibited tumor growth at doses ≥2×10³ pfu/mouse and induced tumor regression at 2×10⁷ pfu/mouse (FIG. 4A). In this model, the hIL12 and hIL7-carrying vaccinia virus did not induce body weight loss compared to the control group (FIG. 4B).

In conclusion, this study indicates that the hIL12 and hIL7-carrying vaccinia virus shows antitumor activities against COLO 741 and U-87 MG xenografts without reducing body weight in immunocompromised mice.

### Example 8. Antitumor Activity of Intratumoral Administration of the hIL12 and hIL7-Carrying Vaccinia Virus-Surrogate in Immunocompetent Mice Subcutaneously Inoculated with CT26.WT Tumor Cells

This study was conducted to investigate antitumor effect of the hIL12 and hIL7-carrying vaccinia virus-surrogate in immunocompetent mice inoculated with murine colorectal carcinoma (CT26.WT) tumor cells. After establishment of the CT26.WT tumor, the hIL12 and hIL7-carrying vaccinia virus-surrogate at a dose range of 2×10⁴ to 2×10⁷ pfu/mouse was intratumorally injected in tumor-bearing mice on days 1, 3 and 5.

On day 18, the hIL12 and hIL7-carrying vaccinia virus-surrogate induced tumor growth inhibition at doses ≥2×10⁵ pfu/mouse; furthermore, 2×10⁷ pfu/mouse of the hIL12 and hIL7-carrying vaccinia virus-surrogate induced 74.1% of tumor regression (FIG. 5A). By day 28, 3 and 5 out of 6 mice achieved CR in the groups treated with the hIL12 and hIL7-carrying vaccinia virus-surrogate at 2×10⁶ pfu/mouse and 2×10⁷ pfu/mouse, respectively. During the study period, there was no obvious difference in body weight between the vehicle control group and the hIL12 and hIL7-carrying vaccinia virus-surrogate groups (FIG. 5B).

In summary, the present study demonstrates an antitumor effect of the hIL12 and hIL7-carrying vaccinia virus-surrogate against a CT26.WT cells in immunocompetent mice.

### Example 9. Antitumor Effects of Intratumoral Administration of the hIL12 and hIL7-Carrying Vaccinia Virus-Surrogate on Days 1 and 8 or Days 1 and 15 in Immunocompetent Mice with CT26.WT Tumor Cells

This study was conducted to assess antitumor effects of the hIL12 and hIL7-carrying vaccinia virus-surrogate administrated on days 1 and 8 or days 1 and 15, against CT26.WT tumors in a syngeneic mouse model.

The hIL12 and hIL7-carrying vaccinia virus-surrogate (2×10⁷ pfu/40 μL/mouse) or vehicle was intratumorally injected into CT26.WT tumor bearing mice on day 1, days 1 and 8 or days 1 and 15. Group 1) vehicle single dose on day 1, Group 2) the hIL12 and hIL7-carrying vaccinia virus-surrogate single dose on day 1, Group 3) the hIL12 and hIL7-carrying vaccinia virus-surrogate 2 doses total (once on day 1 and day 8) and Group 4) the hIL12 and hIL7-carrying vaccinia virus-surrogate 2 doses total (once on day 1 and day 15). Since the mean tumor volume in Group 1 exceeded 2000 mm3, mice in this group were euthanized.

FIG. 6A demonstrates that the hIL12 and hIL7-carrying vaccinia virus-surrogate inhibited tumor growth in all tested groups. FIG. 6B demonstrates that the antitumor efficacy after the administration of the hIL12 and hIL7-carrying vaccinia virus-surrogate on days 1 and 15 was significantly greater than that of the single administration on day 1. There was no significant difference in body weight between the vehicle control group and the hIL12 and hIL7-carrying vaccinia virus-surrogate groups on day 25 (FIG. 6C).

These data show that administration of the hIL12 and hIL7-carrying vaccinia virus-surrogate on days 1 and 15 demonstrates a better antitumor effect compared to a single administration in mice inoculated with CT26.WT tumor cells.

### Example 10. Effect of Intratumoral Administration of the hIL12 and hIL7-Carrying Vaccinia Virus-Surrogate on Immune Responses in Immunocompetent Tumor-Bearing Mice

This study was conducted to investigate the effect of the hIL12 and hIL7-carrying vaccinia virus-surrogate on immune responses in immunocompetent mice subcutaneously inoculated with CT26.WT cells.

After establishment of the tumors, the hIL12 and hIL7-carrying vaccinia virus-surrogate, a recombinant vaccinia virus carrying no immune transgene (Cont-VV) or vehicle was intratumorally injected at a dose of 2×10⁷ pfu/mouse on day 1. The day after the administration, the levels of human IL-7, murine IL-12 and murine IFN-γ in the tumor were measured. In addition, tumor infiltrating lymphocytes were analyzed on day 20 after multiple intratumoral administrations of the hIL12 and hIL7-carrying vaccinia virus-surrogate, Cont-VV or vehicle on days 1, 3 and 5.

The hIL12 and hIL7-carrying vaccinia virus-surrogate significantly increased levels of cytokines, human IL-7, murine IL-12 and murine IFN-γ in the tumors compared to those treated with the vehicle or Cont-VV the day after a single dose (FIG. 7). In addition, the hIL12 and hIL7-carrying vaccinia virus-surrogate induced a significantly higher rate of tumor infiltrating lymphocyte, CD4+ T cells and CD8+ T cells in the tumors compared to those treated with the vehicle or Cont-VV on day 20 after 3 doses (FIG. 8).

These results indicate that intratumoral administration of the hIL12 and hIL7-carrying vaccinia virus-surrogate activates immune responses in immunocompetent mice inoculated with CT26.WT cells.

### Example 11. Time-Course Analysis of Tumor and Serum Cytokine Levels Following the hIL12 and hIL7-Carrying Vaccinia Virus-Surrogate Treatment in Immunocompetent Mice Subcutaneously Inoculated with CT26.WT Tumor Cells

This study was designed to investigate a time-course change in tumor and serum human IL-7, murine IL-12 and murine IFN-γ levels in immunocompetent mice subcutaneously inoculated with CT26.WT tumor cells after intratumoral treatment with the hIL12 and hIL7-carrying vaccinia virus-surrogate.

CT26.WT tumor-bearing mice were treated with the hIL12 and hIL7-carrying vaccinia virus-surrogate at 2×10⁷ pfu/mouse dosing, and tumor and serum samples were collected at 0 h (prior to injection) and 0.5 h, 1 h, 3 h, 6 h, 1 day, 2 days, 3 days, 7 days and 14 days after injection. The values below the limit of quantification were considered 0 for the concentrations. Tumor concentrations of each cytokine were normalized using total protein concentration and expressed as ng/g total protein concentration. The concentration of human IL-7 (A) was determined by ELISA and murine IL-12 (B) and murine IFN-γ (C) were measured by MSD cytokine panel.

As shown in FIGS. 9A and 9B, tumor levels of human IL 7 and murine IL-12 rapidly increased within 0.5 h after treatment and remained elevated for 2 days, after which the levels started to decline. FIG. 9C demonstrates that the production of murine IFN-γ in the tumor began to rise 6 h after treatment, which followed the increases in human IL-7 and murine IL-12. Levels of murine IFN-γ remained elevated until 3 days after treatment and declined thereafter (FIG. 9C). FIG. 10A shows that serum concentrations of human IL-7 were below the limit of quantification (BLQ) for all time points measured except for rapid elevation at 6 h after treatment. The concentration of murine IL-12 in the serum slowly increased during the first 2 days of treatment and peaked between 6 h and 2 days after treatment (FIG. 10B). The concentration of serum murine IFN-γ rapidly increased starting at 6 h and peaked at 1 to 2 days after treatment (FIG. 10C).

These results indicate that the hIL12 and hIL7-carrying vaccinia virus-surrogate treatment leads to transient increases of human IL-7 and murine IL-12 followed by murine IFN-γ production in tumors and sera of CT26.WT tumor-bearing mice.

### Example 12. Analysis of Tumor and Serum Cytokine Levels Following Single and/or Repeated the hIL12 and hIL7-Carrying Vaccinia Virus-Surrogate Treatment in Immunocompetent Mice Subcutaneously Inoculated with CT26.WT Tumor Cells

This study was designed to determine whether tumor and serum human IL-7, murine IL-12 and murine IFN-γ levels increase after single or repeated treatment of the hIL12 and hIL7-carrying vaccinia virus-surrogate in immunocompetent mice subcutaneously inoculated with CT26.WT tumor cells.

The hIL12 and hIL7-carrying vaccinia virus surrogate was injected into CT26.WT tumor-bearing mice with one of the following regimens: (1) single dose of 2×10⁴, 2×10⁵, 2×10⁶ or 2×10⁷ pfu/mouse or (2) repeated dosing of 2×10⁷ pfu/mouse on days 1 and 15. Serum samples were collected from CT26.WT tumor-bearing mice at 0 h (prior to injection) and 0.5 h, 1 h, 3 h, 6 h, 1 day, 2 days, 3 days, 7 days and 14 days after the hIL12 and hIL7-carrying vaccinia virus-surrogate treatment. The values below the limit of quantification were considered 0 for the concentrations. The concentration of human IL-7 (A) was determined by ELISA and murine IL-12 (B) and murine IFN-γ (C) were measured by MSD cytokine panel. Tumor and serum samples were collected from CT26.WT tumor-bearing mice before second dosing (0 h) and at 6 h and 2 days (2 d) after second dosing of the hIL12 and hIL7-carrying vaccinia virus-surrogate. The concentrations of human IL-7, murine IL-12 and murine IFN-γ were determined by MSD V-plex cytokine panels. Mann-Whitney test was used to compare between before (0 h) and 6 hours after second intratumoral injection. The concentrations of murine IFN-γ in serum after 6 h exceeded detection range in 2 out of 10 samples and were assigned upper limit of detection for the concentrations.

At 6 h after a single dose of the hIL12 and hIL7-carrying vaccinia virus-surrogate, tumor concentrations of human IL-7 and murine IL-12 were significantly increased at 2×10⁶ and 2×10⁷ pfu/mouse, and murine IFN-γ production was also significantly elevated at 2×10⁷ pfu/mouse (FIG. 11A). Similarly, concentrations of human IL-7 and murine IL-12 in sera were significantly elevated at 2×10⁷ pfu/mouse, and murine IFN-γ production was significantly increased starting at 2×10⁶ pfu/mouse (FIG. 11A). At 2 days after treatment, tumor levels of human IL-7 and serum murine IFN-γ remained significantly higher than the baseline at 2×10⁷ pfu/mouse (FIG. 11B). Although the levels of murine IL-12 and murine IFN-γ in tumor as well as murine IL-12 in serum were also maintained at the highest dose, the results were not statistically significant (FIG. 11B). However, human IL-7 concentration in serum returned to BLQ 2 days after treatment (FIG. 11B). Furthermore, repeated dosing of the hIL12 and hIL7-carrying vaccinia virus-surrogate at 2×10⁷ pfu/mouse significantly elevated human IL-7, murine IL-12 and murine IFN-γ levels at 6 hours after the second treatment in both tumors and sera (FIG. 12).

### Example 13. Effect of the hIL12 and hIL7-Carrying Vaccinia Virus-Surrogate on Tumor Engraftment after Rechallenge with CT26.WT Tumor Cells in Immunocompetent Mice

This study was conducted to examine whether treatment with the hIL12 and hIL7-carrying vaccinia virus-surrogate induces long-lasting immune memory in mice subcutaneously inoculated with CT26.WT cells.

The hIL12 and hIL7-carrying vaccinia virus-surrogate was intratumorally injected in CT26.WT-tumor-bearing mice at 2×10⁷ pfu/mouse on days 1, 3 and 5. The hIL12 and hIL7-carrying vaccinia virus-surrogate induced CR in 26 out of 30 mice until 23 days after the completion of the treatment with the hIL12 and hIL7-carrying vaccinia virus-surrogate. Ninety days after the completion of intratumoral injection of the hIL12 and hIL7-carrying vaccinia virus-surrogate, the mice that had achieved CR were subcutaneously rechallenged with CT26.WT cells at 5×10⁵ cells/mouse (n=10) and were observed for 28 days after the inoculation.

At that time of re-challenge, all 26 mice that had prior CR associated with the hIL12 and hIL7-carrying vaccinia virus-surrogate treatment were alive until 90 days after the final injection of the hIL12 and hIL7-carrying vaccinia virus-surrogate, with no significant difference in body weight compared to age-matched control mice (FIG. 13). After the rechallenge with CT26.WT cells, 9 out of 10 mice that had achieved CR on the hIL12 and hIL7-carrying vaccinia virus-surrogate remained tumor-free, whereas all treatment-naïve mice developed tumors within 28 days (FIG. 14).

In conclusion, these results suggest that immunocompetent mice that had experienced CR on the hIL12 and hIL7-carrying vaccinia virus-surrogate developed long-term antitumor immune memory against CT26.WT tumor cells.

### Example 14. Abscopal Antitumor Effect of Intratumoral Administration of the hIL12 and hIL7-Carrying Vaccinia Virus-Surrogate in Immunocompetent Mice Bilaterally Inoculated with CT26.WT Tumor Cells

This study was conducted to investigate the abscopal antitumor effect of the hIL12 and hIL7-carrying vaccinia virus-surrogate in immunocompetent mice bilaterally inoculated with CT26.WT tumor cells.

CT26.WT tumor cells were subcutaneously inoculated into both the right and left flanks of mice. After tumors were established on both sides of the mice, the hIL12 and hIL7-carrying vaccinia virus-surrogate, Cont-VV or vehicle was injected into the unilateral tumor on days 1, 3 and 5. Statistical analysis was performed using the values of tumor volumes (A: injected tumors, B: uninjected tumors) or body weight (C) on day 17.

On day 17, the hIL12 and hIL7-carrying vaccinia virus-surrogate inhibited tumor growth by 96% and 64% in the injected and the contralateral uninjected tumors, respectively (FIGS. 15A and 15B). Cont-VV inhibited tumor growth by 70% in the injected tumors; however, it did not show antitumor effect on the uninjected tumors. By day 28, 8 out of 10 mice achieved CR of the injected tumors and 1 out of 10 mice achieved CR of the uninjected tumors in the hIL12 and hIL7-carrying vaccinia virus-surrogate treated group. The average body weight of mice in the hIL12 and hIL7-carrying vaccinia virus-surrogate group gradually increased during the study period, although the body weight was significantly lower than that of vehicle-treated mice at day 17, which is assumed to be due to the decreased size of tumors after the administration of the hIL12 and hIL7-carrying vaccinia virus-surrogate (FIG. 15C).

In conclusion, this study indicates the hIL12 and hIL7-carrying vaccinia virus-surrogate has an abscopal antitumor effect against the uninjected tumors in mice inoculated with CT26.WT tumor cells.

### Example 15. Antitumor Effect of the hIL12 and hIL7-Carrying Vaccinia Virus-Surrogate in Combination with Immune Checkpoint Inhibitors in Immunocompetent Mice Bilaterally Inoculated with CT26.WT Tumor Cells

This study was conducted to investigate the antitumor effect of the hIL12 and h1L7-carrying vaccinia virus-surrogate in combination with immune checkpoint inhibitors, anti-PD-1 Ab or anti-CTLA4 Ab, in immunocompetent mice bilaterally inoculated with CT26.WT tumor cells.

After the establishment of tumors, vehicle solution or 2×10⁷ pfu/mouse of the hIL12 and hIL7-carrying vaccinia virus-surrogate was injected into the unilateral tumor on days 1, 3 and 6. On day 6, phosphate buffered saline or anti-PD-1 antibody (100 μg/mouse) or anti-CTLA4 antibody (200 μg/mouse) was administered intraperitoneally twice weekly. Mice in vehicle, anti-PD-1 antibody monotherapy and anti-CTLA-4 Ab monotherapy group were euthanized on day 24 since the average of tumor volumes in the groups exceeded 2000 mm3 on both flanks.

In the model, anti-PD-1 antibody or anti-CTLA4 antibody monotherapy did not show significant antitumor activity in injected and uninjected tumors. In the virus-injected tumor sites, the hIL12 and hIL7-carrying vaccinia virus-surrogate alone, the combination of the hIL12 and hIL7-carrying vaccinia virus-surrogate with anti-PD-1 antibody and the combination of the hIL12 and hIL7-carrying vaccinia virus-surrogate with anti-CTLA4 Ab induced CR in 9 out of 10, 10 out of 10 and 9 out of 10 mice on day 37, respectively. In the uninjected tumors, 6 out of 10 and 4 out of 10 mice achieved CR in the group treated with the combination of the hIL12 and hIL7-carrying vaccinia virus-surrogate with anti-PD-1 antibody or anti-CTLA4 antibody, respectively, while only 1 out of 10 mice achieved CR in the group treated with the hIL12 and hIL7-carrying vaccinia virus-surrogate alone (FIG. 16).

In conclusion, this result indicates the combination of the hIL12 and hIL7-carrying vaccinia virus-surrogate with either anti-PD-1 or anti-CTLA4 antibodies demonstrates higher antitumor efficacy than any of the 3 agents administered as monotherapy

### Summary of Examples 1-15

The hIL12 and hIL7-carrying vaccinia virus is a replication-competent vaccinia virus incorporating transgenes for human IL-12 and IL-7. The hIL12 and hIL7-carrying vaccinia virus was designed based on a vaccine strain, LC16mO, with further modifications consisting of functional deletion of VGF and O1L, by insertion of human IL-12 and human IL-7, respectively and modification of B5R (U.S. Patent Publication No. 2017/0340687, the entire contents of which are incorporated herein by reference).

In in vitro studies, the hIL12 and hIL7-carrying vaccinia virus demonstrated cytotoxicity in various types of human cancer cells including lung, kidney, bladder, head and neck, breast, ovary, esophageal, gastric, colon, colorectal, liver, bile duct, pancreatic, prostate and cervical cancer and glioblastoma, neuroblastoma, myeloma and melanoma. In in vivo studies, the hIL12 and hIL7-carrying vaccinia virus induced tumor regression against human colorectal carcinoma and glioblastoma following intratumoral injection in immunocompromised mice. These results demonstrate a broad spectrum of direct oncolytic activity of the hIL12 and hIL7-carrying vaccinia virus against human cancer cells. In addition, secretion of human IL-12 and human IL-7 proteins was confirmed in several types of human cancer cells treated with the hIL12 and hIL7-carrying vaccinia virus. In the viral genome of the hIL12 and hIL7-carrying vaccinia virus, VGF and O1L are functionally deleted. VGF and O1L are virulence factors that are involved in sustained activation of the Raf/MEK/ERK signaling pathway to promote viral virulence in the infected cells (Schweneker et al, J Virol. 2012; 86:2323-36). Replication of the hIL12 and hIL7-carrying vaccinia virus genome was more selective in human cancer cells than in normal cells, suggesting that this selectivity is due to the functional deletion of VGF and O1L in the hIL12 and hIL7-carrying vaccinia virus. In the studies using immunocompetent mice, the hIL12 and hIL7-carrying vaccinia virus-surrogate, which carries murine IL-12 instead of human IL-12, was used to estimate the immune activation profile of the hIL12 and hIL7-carrying vaccinia virus, as it is known that human IL-12 is not cross-reactive in mouse immune cells (Schweneker et al, supra). The structure of the hIL12 and hIL7-carrying vaccinia virus-surrogate is the same as that of the hIL12 and hIL7-carrying vaccinia virus except for the species derivation of the IL-12 transgene. It is assumed that the hIL12 and hIL7-carrying vaccinia virus-surrogate, in which murine IL-12 and human IL-7 insertionally inactivate VGF and O1L, also replicates in cancer cells more selectively than in normal cells. In addition, the hIL12 and hIL7-carrying vaccinia virus-surrogate was confirmed to show cytotoxic activity against human cancer cells and induce secretion of murine IL-12 and human IL-7 proteins from the infected cancer cells similarly as the hIL12 and hIL7-carrying vaccinia virus did, indicating that the hIL12 and hIL7-carrying vaccinia virus-surrogate can estimate the antitumor activity of the hIL12 and hIL7-carrying vaccinia virus as a surrogate virus.

Repeated intratumoral injection of the hIL12 and hIL7-carrying vaccinia virus-surrogate showed significant antitumor activity in an immunocompetent mouse model. In the same model, administration of the hIL12 and hIL7-carrying vaccinia virus-surrogate on days 1 and 15 showed superior efficacy compared to a single administration, suggesting that repeated administration may be efficacious in cancer patients. IL-12 is known to activate both innate and adaptive immunity partially due to IFN-γ secretion from natural killer cells, CD8⁺ T cells and CD4⁺ T cells. IL-7 is crucial for T-cell homeostasis and known to show synergistic stimulatory activity to T cells when combined with IL-12 (Mehrotra et al, J Immunol. 1995; 154:5093-102). The hIL12 and hIL7-carrying vaccinia virus-surrogate induced intratumoral secretion of murine IL-12, human IL-7 and IFN-γ proteins and increased tumor infiltration of CD8+ T cells and CD4⁺ T cells, suggesting that intratumoral expression of IL-12 and IL-7 mediated by the oncolytic vaccinia virus has a function to upregulate immune responses in the tumor microenvironment resulting in antitumor efficacy. In the time-course experiment, the transient increases in all observed tumor and serum cytokine levels declined to close to basal levels as it was observed on day 3 and day 14 after the treatment. In addition, the hIL12 and hIL7-carrying vaccinia virus-surrogate showed an abscopal effect in a bilateral tumor model, in which treatment of the hIL12 and hIL7-carrying vaccinia virus-surrogate into the unilateral tumor led to significant antitumor effect in both the injected and the contralateral uninjected tumors, indicating that local immune activation in the virus-injected tumor affected the uninjected distant tumors. Furthermore, mice that had achieved CR by the hIL12 and hIL7-carrying vaccinia virus-surrogate capably rejected the same cancer cells after rechallenge about 90 days after the CR, suggesting establishment of antitumor immune memory by the hIL12 and hIL7-carrying vaccinia virus-surrogate. In this bilateral tumor model, the administration of the hIL12 and hIL7-carrying vaccinia virus-surrogate prior to anti-PD-1 or anti-CTLA4 Ab treatment demonstrated superior efficacy to any of the 3 agents administered alone, suggesting combination treatment may be effective in patients with solid tumors.

In these studies, the lack of obvious weight changes following administration of the hIL12 and hIL7-carrying vaccinia virus-surrogate indicates no overt signs of autoimmunity, although the potential risk for autoimmune reaction should be closely monitored for in the clinical setting.

Taken together, the hIL12 and hIL7-carrying vaccinia virus is intended to replicate selectively in tumor tissues resulting in tumor destruction and expression of immunomodulators leading to immune activation in the tumor microenvironment as well as potentially inducing a systemic antitumor activity. The hIL12 and hIL7-carrying vaccinia virus may show anticancer activities via direct cell lysis of tumor cells and via immune-mediated cancer cell destruction in a variety of tumor types.

### Examples 16-19

The following methods were used in the biodistribution and shedding studies provided in Examples 16-19.

Biodistribution and shedding studies in mice and cynomolgus monkeys were conducted. the hIL12 and hIL7-carrying vaccinia virus and the hIL12 and hIL7-carrying vaccinia virus-surrogate were analyzed by qPCR. Both the hIL12 and hIL7-carrying vaccinia virus and the hIL12 and hIL7-carrying vaccinia virus-surrogate share common DNA sequences. The primer pair and probe specific for the detection of the common DNA sequences were used for the quantification of the hIL12 and hIL7-carrying vaccinia virus and the hIL12 and hIL7-carrying vaccinia virus-surrogate viral genome numbers. The range of the calibration curve was 100 to 1×10⁷ (Viral genomes (vg)/μg DNA in mice and 125 to 2.5×10⁷ vg/μg DNA in cynomolgus monkeys. The limit of detection was 50 vg/μg DNA in mice and 31.25 vg/μg DNA in cynomolgus monkeys. The analytical method has sufficient specificity, as well as within-run and between-run accuracy and precision.

### Example 16. Biodistribution and Shedding of the hIL12 and hIL7-Carrying Vaccinia Virus in Normal Mice

The hIL12 and hIL7-carrying vaccinia virus was administered as a single intravenous dose to male and female CD-1 mice at 8.5×10⁹ pfu/kg. As shown in Table 7, the hIL12 and hIL7-carrying vaccinia virus DNA was detected in blood for at least 28 days after administration and was not detected in any animal at 84 days after administration. The hIL12 and hIL7-carrying vaccinia virus DNA was detected in all tissues examined except brain. The hIL12 and hIL7-carrying vaccinia virus DNA in tissues decreased time dependently in tissues and was BLQ at 14 days after administration. Tissues presenting the highest level of the hIL12 and hIL7-carrying vaccinia virus DNA were the liver, lung and spleen. The hIL12 and hIL7-carrying vaccinia virus DNA excreted in urine or feces during the study was BLQ. No remarkable sex differences were observed.

### Example 17. Biodistribution and Shedding of the hIL12 and hIL7-Carrying Vaccinia Virus-Surrogate in Tumor Bearing Mice

The hIL12 and hIL7-carrying vaccinia virus-surrogate was administered as a single intratumoral injection to male and female tumor-bearing BALB/c mice at 2×10⁷ pfu/mouse. As shown in Table 8, the hIL12 and hIL7-carrying vaccinia virus-surrogate DNA was detected in tumors and decreased time dependently. The hIL12 and hIL7-carrying vaccinia virus-surrogate DNA was BLQ in tumors at 14 days after administration, except for 1 of 5 animals. The hIL12 and hIL7-carrying vaccinia virus-surrogate DNA was BLQ in the blood, brain, heart, kidney, lung, feces, ovary and urine. The hIL12 and hIL7-carrying vaccinia virus-surrogate DNA was detected in the following tissues: iliac lymph node, spleen, testis and uterus at 4 hours after administration in 1 of 5 animals for each tissue; at 1 day after administration, the hIL12 and hIL7-carrying vaccinia virus-surrogate DNA was detected in liver tissue of 1 of 5 animals. The hIL12 and hIL7-carrying vaccinia virus-surrogate DNA was not detected in these tissues at later time points (1 day or 3 to 14 days after administration). No remarkable sex differences were observed.

With the exception of the tumor and iliac lymph node, human IL-7 and murine IL-12 were measured in tissues from those animals in which the hIL12 and hIL7-carrying vaccinia virus-surrogate DNA was detected. Human IL-7 and murine IL-12 were BLQ in the tissues examined.

### Example 18. Determination of the hIL12 and hIL7-Carrying Vaccinia Virus-Surrogate in Skin Swabs of Tumor Bearing Mice

The hIL12 and hIL7-carrying vaccinia virus-surrogate was administered once intratumorally to male and female tumor-bearing BALB/c mice at 2×10⁷ pfu/mouse. As shown in Table 9, the hIL12 and hIL7-carrying vaccinia virus-surrogate was detected in skin swabs at the injection site immediately after administration (within 2 minutes). The hIL12 and hIL7-carrying vaccinia virus-surrogate decreased time dependently and was BLQ at 3 days and later time points up to 21 days after administration. No remarkable sex differences were observed.

### Example 19. Biodistribution and Shedding of the hIL12 and hIL7-Carrying Vaccinia Virus in Cynomolgus Monkeys

The hIL12 and hIL7-carrying vaccinia virus was administered intravenously to male and female cynomolgus monkeys at 3.4×10⁸ and 3.4×10⁹ pfu/kg once weekly for 4 weeks.

As shown in Table 10, the hIL12 and hIL7-carrying vaccinia virus DNA was detected in blood and decreased time dependently. At 3.4×10⁸ pfu/kg, the hIL12 and hIL7-carrying vaccinia virus DNA was BLQ in blood 3 days or later time points after administration. At 3.4×10⁹ pfu/kg, the hIL12 and hIL7-carrying vaccinia virus DNA was detected for 7 days after administration. The hIL12 and hIL7-carrying vaccinia virus DNA in blood increased with increasing dose.

In tissues, the hIL12 and hIL7-carrying vaccinia virus DNA was detected only in spleen at 7 days after the fourth administration.

At 3.4×10⁸ pfu/kg, the hIL12 and hIL7-carrying vaccinia virus DNA was BLQ in oral swab samples, lacrimal swab samples, urine or feces during the study. At 3.4×10⁹ pfu/kg, the hIL12 and hIL7-carrying vaccinia virus DNA was detected in oral swab samples at 4 hours and 1 day after administration and feces at 3 days after administration. the hIL12 and hIL7-carrying vaccinia virus DNA in oral swab samples and feces was not detected 7 days after the first or second administration. The hIL12 and hIL7-carrying vaccinia virus DNA was BLQ in lacrimal swab samples or urine during the study.

Overall, no remarkable sex differences were observed in biodistribution and shedding in cynomolgus monkeys.

TABLE 7
Biodistribution and Shedding after a Single Intravenous Dose in Normal Mice (qPCR)

| | |
|---|---|
| Species/Strain | Mouse/Swiss, CD-1 |
| Gender (M/F)/ | M and F/5 each per time point |
| Number of animals | |
| Feeding condition | Nonfasted |
| Administered drug | The hIL12 and hIL7-Carrying Vaccinia Virus |
| Vehicle/Formulation | 30 mmol/L Tris HCl, 10% sucrose, pH 7.6 |
| Method of administration | intravenous |
| Assay | qPCR |
| Sampling time | 4 h, 1, 3, 7, 14, 28 and 84 days after administration |

| | qPCR Measurement (geometric mean vg number/μg DNA) | | | | | | | |
|---|---|---|---|---|---|---|---|---|
| | Dose (pfu/kg) | | | | | | | |
| | 0 | | 8.5 × 10⁹ | | | | | |
| | Time after Dosing | | | | | | | |
| | 1 day (24 h) | | 4 h | | 1 day (24 h) | | 3 days (72 h) | |
| | Animals | | | | | | | |
| | 5/M | 5/F | 5/M | 5/F | 5/M | 5/F | 5/M | 5/F |
| Blood | BLQ | BLQ | 3.64E+03¶ | 4.77E+03¶ | 2.44E+03‡ | 1.45E+03 | 3.49E+03 | 4.26E+03¶ |
| Brain | BLQ | BLQ | BLQ | BLQ | BLQ | BLQ | BLQ | BLQ |
| Heart | BLQ | BLQ | 7.20E+02 | 5.36E+02¶ | 2.06E+02‡ | 4.52E+02 | 5.27E+02 | 6.38E+02‡ |
| Kidneys | BLQ | BLQ | 2.37E+02 | 3.69E+02¶ | 2.99E+02† | 9.49E+02 | BLQ | 6.63E+02† |
| Liver | BLQ | BLQ | 4.30E+04 | 1.89E+04 | 4.61E+03‡ | 1.58E+04 | 5.01E+03 | 1.39E+03¶ |
| Lungs | BLQ | BLQ | 5.40E+03 | 2.58E+03 | 1.48E+03‡ | 2.51E+03 | 4.67E+02 | 1.24E+03¶ |
| Mesenteric | BLQ | BLQ | 1.50E+02‡ | 1.30E+02§ | BLQ | BLQ | BLQ | BLQ |
| lymph nodes | | | | | | | | |
| Spleen | BLQ | BLQ | 1.21E+05 | 3.51E+04 | 2.65E+03‡ | 1.00E+04 | 1.75E+03 | 1.37E+03§ |
| Testes | BLQ | NA | BLQ | NA | BLQ | NA | 2.81E+02† | NA |
| Ovaries | NA | BLQ | NA | 3.25E+02¶ | NA | 2.11E+03 | NA | BLQ |
| Uterus | NA | BLQ | NA | BLQ | NA | 5.08E+02‡ | NA | BLQ |
| Urine^{a} | BLQ | BLQ | NA | NA | BLQ | BLQ | BLQ | BLQ |
| Feces^{a} | BLQ | BLQ | NA | NA | BLQ | BLQ | BLQ | BLQ |

| | qPCR Measurement (geometric mean vg number/μg DNA) | | | | | | | |
|---|---|---|---|---|---|---|---|---|
| | Dose (pfu/kg) | | | | | | | |
| | 8.5 × 10⁹ | | | | | | | |
| | Time after Dosing | | | | | | | |
| | 7 days (168 h) | | 14 days | | 28 days | | 84 days | |
| | Animals | | | | | | | |
| | 5/M | 5/F | 5/M | 5/F | 5/M | 5/F | 5/M | 5/F |
| Blood | 3.21E+03 | 2.27E+04§ | 2.72E+03 | 4.98E+03 | 2.13E+03¶ | 9.70E+02 | BLQ | BLQ |
| Brain | BLQ | BLQ | BLQ | BLQ | BLQ | BLQ | BLQ | BLQ |
| Heart | 1.76E+02‡ | 1.87E+02† | BLQ | BLQ | BLQ | BLQ | BLQ | BLQ |
| Kidneys | BLQ | BLQ | BLQ | BLQ | BLQ | BLQ | BLQ | BLQ |
| Liver | BLQ | BLQ | BLQ | BLQ | BLQ | BLQ | BLQ | BLQ |
| Lungs | 7.00E+02† | BLQ | BLQ | BLQ | BLQ | BLQ | BLQ | BLQ |
| Mesenteric | BLQ | BLQ | BLQ | BLQ | BLQ | BLQ | BLQ | BLQ |
| lymph nodes | | | | | | | | |
| Spleen | BLQ | BLQ | BLQ | BLQ | BLQ | BLQ | BLQ | BLQ |
| Testes | BLQ | NA | BLQ | NA | BLQ | NA | BLQ | NA |
| Ovaries | NA | BLQ | NA | BLQ | NA | BLQ | NA | BLQ |
| Uterus | NA | BLQ | NA | BLQ | NA | BLQ | NA | BLQ |
| Urine^{a} | BLQ | BLQ | BLQ | BLQ | BLQ | BLQ | BLQ | BLQ |
| Feces^{a} | BLQ | BLQ | BLQ | BLQ | BLQ | BLQ | BLQ | BLQ |

Additional information: None
Numerical data are expressed as geometric mean values unless otherwise specified.
BLQ: below the limit of quantification (<100 vg/μg DNA); F: female; M: male; NA: not applicable; qPCR: quantitative polymerase chain reaction.
^{a}Urine and feces from each housing group were pooled and analyzed, respectively.
†Numerical data in 1 animal, BLQ in 4 animals.
‡Numerical data in 2 animals, BLQ in 3 animals.
§Numerical data in 3 animals, BLQ in 2 animals.
¶Numerical data in 4 animals, BLQ in 1 animal.

TABLE 8
Biodistribution and Shedding after a Single Intratumoral Dose in Tumor-bearing Mice (qPCR)

| | |
|---|---|
| Species/Strain | Mouse/BALB/c |
| Gender (M/F)/ | M and F/5 each per time point |
| Number of animals | |
| Feeding condition | Nonfasted |
| Administered drug | The hIL12 and hIL7-Carrying Vaccinia Virus-Surrogate |
| Vehicle/Formulation | 30 mmol/L Tris-HCl, 10% sucrose, pH 7.6 |
| Method of administration | Intratumoral |
| Dose (pfu/mouse) | 2 × 10⁷ |
| Dose (pfu/mL) | 6.67 × 10⁸ |
| Assay | qPCR |
| Sampling time | 4 h, 1, 3, 7 and 14 days after administration |

| | qPCR Measurement (geometric mean vg number/μg DNA) | | | | | | | | | |
|---|---|---|---|---|---|---|---|---|---|---|
| | Time after Dosing | | | | | | | | | |
| | 4 h | | 1 day | | 3 days | | 7 days | | 14 days | |
| | Number of Animals | | | | | | | | | |
| | 5/M | 5/F | 5/M | 5/F | 5/M | 5/F | 5/M | 5/F | 5/M | 5/F |
| Tumor | 1.15E+06 | 1.50E+06 | 1.04E+05 | 7.66E+05 | 2.67E+05 | 1.42E+05 | 1.01E+04 | 2.05E+04‡ | 1.06E+05§** | BLQ¶ |
| Blood | BLQ | BLQ | BLQ** | BLQ | BLQ | BLQ | BLQ | BLQ | BLQ | BLQ |
| Brain | BLQ | BLQ | BLQ | BLQ | BLQ | BLQ | BLQ | BLQ | BLQ | BLQ |
| Heart | BLQ* | BLQ | BLQ | BLQ | BLQ | BLQ | BLQ | BLQ | BLQ | BLQ |
| Kidneys | BLQ | BLQ | BLQ | BLQ | BLQ | BLQ | BLQ | BLQ | BLQ | BLQ |
| Liver | BLQ | BLQ | 1.08E+03† | BLQ | BLQ | BLQ | BLQ | BLQ | BLQ | BLQ |
| Lungs | BLQ | BLQ | BLQ | BLQ | BLQ | BLQ | BLQ | BLQ | BLQ | BLQ |
| Iliac | 1.24E+02† | BLQ | BLQ | BLQ | BLQ | BLQ | BLQ | BLQ | BLQ | BLQ |
| lymph nodes | | | | | | | | | | |
| Spleen | 1.16E+02† | BLQ | BLQ | BLQ | BLQ | BLQ | BLQ | BLQ | BLQ | BLQ |
| Testes | 2.71E+02† | NA | BLQ | NA | BLQ | NA | BLQ | NA | BLQ | NA |
| Ovaries | NA | BLQ | NA | BLQ | NA | BLQ | NA | BLQ | NA | BLQ |
| Uterus | NA | 7.48E+02† | NA | BLQ | NA | BLQ | NA | BLQ | NA | BLQ |
| Urine | NA | NA | BLQ | BLQ | BLQ | BLQ | BLQ | BLQ | BLQ | BLQ |
| Feces | NA | NA | BLQ | BLQ | BLQ | BLQ | BLQ | BLQ | BLQ | BLQ |

Additional information: With the exception of the tumor and iliac lymph node, human IL-7 and murine IL-12
were measured in tissues from those animals in which the hIL12 and hIL7-carrying vaccinia virus-surrogate was
detected. Human IL-7 and murine IL-12 were BLQ in the tissues examined.
Numerical data are expressed as geometric mean values unless otherwise specified.
BLQ: below the limit of quantification (<100 vg/μg DNA); F: female; IL-7: interleukin-7; IL-12: interleukin-12; M: male; NA: not applicable; qPCR: quantitative polymerase chain reaction.
*BLQ in 4 animals, not reproducible data even after repetition in 1 animal.
**<1 μg of DNA were analyzed.
†Numerical data in 1 animal, BLQ in 4 animals.
‡Numerical data in 4 animals, BLQ in 1 animal.
§Number of 4 animals (numerical data in 1 animal, BLQ in 3 animals) because the tumor in 1 animal was not found visually at the time of sampling.
¶Number of 4 animals because the tumor in 1 animal was not found visually at the time of sampling.

TABLE 9
Mean Number of Viral Genomes in Skin Swab after a Single Intratumoral Administration
of the hIL12 and hIL7-Carrying Vaccinia Virus-surrogate to Tumor-bearing Mice

| | |
|---|---|
| Species/Strain | Mouse/BALB/c |
| Gender (M/F)/ | M and F/5 each per time point |
| Number of animals | |
| Feeding condition | Non-fasted |
| Administered drug | The hIL12 and hIL7-Carrying Vaccinia Virus-Surrogate |
| Vehicle/Formulation | 30 mmol/L Tris-HCl, 10% sucrose, pH 8.0 |
| Method of administration | Intratumoral injection |
| Dose (pfu/mouse) | 2 × 10⁷ |
| Assay | qPCR |
| Sampling time | pre, within 2 min, 4 h, 1, 3, 7, 14 and 21 days after administration |

| | qPCR Measurement (geometric mean vg number/μg DNA) | | | | | | |
|---|---|---|---|---|---|---|---|
| | Time after dosing | | | | | | |
| | pre | | within 2 min | | 4 h | | 1 day |
| | Gender | | | | | | |
| | M | F | M | F | M | F | M |
| Skin swab | BLQ | BLQ | 1.37E+08 ‡ | 7.35E+06 § | 1.20E+07 † | 9.43E+05 ‡ | 2.07E+06 † |

Additional information: None

| | qPCR Measurement (geometric mean vg number/μg DNA) | | | | | | | | |
|---|---|---|---|---|---|---|---|---|---|
| | Time after dosing | | | | | | | | |
| | 1 day | 3 days | | 7 days | | 14 days | | 21 days | |
| | Gender | | | | | | | | |
| | F | M | F | M | F | M | F | M | F |
| Skin swab | BLQ | BLQ | BLQ | BLQ | BLQ | BLQ | BLQ | BLQ | BLQ |

Additional information: None
Numerical data are expressed as geometric mean values unless otherwise specified.
BLQ: below the limit of quantification (<100 vg/μg DNA); F: female; M: male; qPCR: quantitative polymerase chain reaction.
† Numerical data in 2 animals, BLQ in 3 animals.
‡ Numerical data in 3 animals, BLQ in 2 animals.
§ Numerical data in 4 animals, BLQ in 1 animal.

TABLE 10
Biodistribution and Shedding after Repeated Intravenous Doses in Cynomolgus Monkeys (qPCR)

| | |
|---|---|
| Species/Strain | Cynomolgus monkey |
| Gender (M/F)/ | M and F/3 |
| Number of animals | |
| Feeding condition | Nonfasted |
| Administered drug | The hIL12 and hIL7-Carrying Vaccinia Virus |
| Vehicle/Formulation | 30 mmol/L Tris-HCl, 10% sucrose, pH 7.6 |
| Method of administration | Intravenous |
| Assay | qPCR |
| Sampling time | 1 h, 4 h, 1, 3, 4, 7, 14, 21 and 28 days after the first administration |

| | | | qPCR Measurement (geometric mean vg number/μg DNA) | | | | | |
|---|---|---|---|---|---|---|---|---|
| | | | Time after Dosing | | | | | |
| | | Dose | 1 h | | 4 h | | 1 day (24 h) | |
| | Group§ | (pfu/kg) | 3/M | 3/F | 3/M | 3/F | 3/M | 3/F |
| Blood | 3 | 3.4 × 10⁸ | 2.41E+03 | 1.76E+03 | 5.92E+02 | 4.43E+03 | 1.82E+02‡ | 3.22E+02† |
| | 4 | 3.4 × 10⁹ | NA | NA | 9.45E+04 | 2.54E+05 | 2.62E+04 | 8.25E+04 |
| Oral swab | 3 | 3.4 × 10⁸ | BLQ | BLQ | BLQ | BLQ | BLQ | BLQ |
| | 4 | 3.4 × 10⁹ | NA | NA | 5.85E+02‡ | 2.41E+03† | 1.49E+02†^{b} | 1.09E+03‡^{b} |
| Lacrimal | 3 | 3.4 × 10⁸ | BLQ | BLQ | BLQ | BLQ | BLQ | BLQ |
| sample | 4 | 3.4 × 10⁹ | NA | NA | BLQ | BLQ | BLQ^{b} | BLQ^{b} |
| Urine | 3 | 3.4 × 10⁸ | NA | NA | NA | NA | BLQ | BLQ |
| | 4 | 3.4 × 10⁹ | NA | NA | NA | NA | BLQ^{b} | BLQ^{b} |
| Feces | 3 | 3.4 × 10⁸ | NA | NA | NA | NA | BLQ^{f} | BLQ^{f} |
| | 4 | 3.4 × 10⁹ | NA | NA | NA | NA | BLQ^{b} | BLQ^{b} |

| | | | qPCR Measurement (geometric mean vg number/μg DNA) | | | |
|---|---|---|---|---|---|---|
| | | | Time after Dosing | | | |
| | | Dose | 3 days (72 h) | | 4 days (96 h) | |
| | Group§ | (pfu/kg) | 3/M | 3/F | 3/M | 3/F |
| Blood | 3 | 3.4 × 10⁸ | BLQ | BLQ | BLQ | BLQ |
| | 4 | 3.4 × 10⁹ | 7.32E+02 | 1.93E+03 | 9.36E+02^{a} | 1.51E+03 |
| Oral swab | 3 | 3.4 × 10⁸ | BLQ | BLQ | BLQ | BLQ |
| | 4 | 3.4 × 10⁹ | BLQ | BLQ | BLQ^{d} | BLQ |
| Lacrimal | 3 | 3.4 × 10⁸ | BLQ | BLQ | BLQ | BLQ |
| sample | 4 | 3.4 × 10⁹ | BLQ | BLQ | BLQ | BLQ |
| Urine | 3 | 3.4 × 10⁸ | BLQ^{a} | BLQ | NA | NA |
| | 4 | 3.4 × 10⁹ | BLQ^{e} | BLQ | NA | NA |
| Feces | 3 | 3.4 × 10⁸ | BLQ | BLQ | NA | NA |
| | 4 | 3.4 × 10⁹ | BLQ | 1.40E+03†^{e} | NA | NA |

| | | | qPCR Measurement (geometric mean vg number/μg DNA) | | | | | | | |
|---|---|---|---|---|---|---|---|---|---|---|
| | | | Time after Dosing | | | | | | | |
| | | | 7 days | | 14 days | | 21 days | | 28 days | |
| | | | (before the | | (before the | | (before the | | (7 days after | |
| | | Dose | second dose) | | third dose^{g}) | | fourth dose) | | fourth dose) | |
| | Group§ | (pfu/kg) | 3/M | 3/F | 3/M | 3/F | 3/M | 3/F | 3/M | 3/F |
| Blood | 3 | 3.4 × 10⁸ | BLQ | BLQ | BLQ | BLQ | NA | NA | BLQ | BLQ |
| | 4 | 3.4 × 10⁹ | 5.78E+02† | 7.43E+02‡ | BLQ^{c} | 3.77E+02 | NA | NA | NA | NA |
| Oral swab | 3 | 3.4 × 10⁸ | BLQ | BLQ | BLQ | BLQ | BLQ | BLQ | BLQ | BLQ |
| | 4 | 3.4 × 10⁹ | BLQ | BLQ | BLQ^{c} | BLQ | NA | NA | NA | NA |
| Lacrimal | 3 | 3.4 × 10⁸ | BLQ | BLQ | BLQ | BLQ | BLQ | BLQ | BLQ | BLQ |
| sample | 4 | 3.4 × 10⁹ | BLQ | BLQ | BLQ^{c} | BLQ | NA | NA | NA | NA |
| Urine | 3 | 3.4 × 10⁸ | BLQ | BLQ | BLQ | BLQ | BLQ | BLQ | BLQ | BLQ |
| | 4 | 3.4 × 10⁹ | BLQ | BLQ | BLQ^{c} | BLQ | NA | NA | NA | NA |
| Feces | 3 | 3.4 × 10⁸ | BLQ | BLQ | BLQ | BLQ | BLQ | BLQ | BLQ | BLQ |
| | 4 | 3.4 × 10⁹ | BLQ | BLQ | BLQ^{c} | BLQ | NA | NA | NA | NA |

| | | | qPCR Measurement (geometric mean vg number/μg DNA) | |
|---|---|---|---|---|
| | | | Time after Dosing | |
| | | | 28 days (7 days after fourth dose) | |
| | | Dose | Number of Animals | |
| | Group§ | (pfu/kg) | 3/M | 3/F |
| Brain | 3 | 3.4 × 10⁸ | BLQ | BLQ |
| Heart | | | BLQ | BLQ |
| Kidneys | | | BLQ | BLQ |
| Liver | | | BLQ | BLQ |
| Lungs | | | BLQ | BLQ |
| Lymph nodes: | | | BLQ | BLQ |
| mandibular | | | | |
| Lymph nodes: | | | BLQ | BLQ |
| mesenteric | | | | |
| Ovaries | | | NA | BLQ |
| Spleen | | | 2.21E+03 | 9.20E+02‡ |
| Testes | | | BLQ | NA |
| Uterus | | | NA | BLQ |

Additional information: None
Numerical data are expressed as geometric mean values unless otherwise specified.
BLQ: below the limit of quantification (<125 vg/μg DNA); F: female; M: male; NA: not applicable; qPCR: quantitative polymerase chain reaction.
^{a}One sample was missing.
^{b}Sampled at 45 h after the first administration.
^{c}One animal was sacrificed before the designated sampling point.
^{d}One sample was lost.
^{e}Including 1 sample at 96 h after the first administration.
^{f}n = 1.
^{g}At sacrifice for group 4 animals.
§Data BLQ in all Group 2 animals.
†Numerical data in 1 animal, BLQ in 2 animals.
‡Numerical data in 2 animals, BLQ in 1 animal.

### Summary of Examples 16-19

When the hIL12 and hIL7-carrying vaccinia virus was administered as a single intravenous dose to mice at 8.5×10⁹ pfu/kg, the hIL12 and hIL7-carrying vaccinia virus DNA was detected in blood for at least 28 days after administration and was not detected in any animal at 84 days after administration. The hIL12 and hIL7-carrying vaccinia virus DNA was detected in all tissues examined except brain. The hIL12 and hIL7-carrying vaccinia virus DNA in tissues decreased time dependently and was BLQ at 14 days after administration. The hIL12 and hIL7-carrying vaccinia virus DNA was BLQ in urine or feces. No remarkable sex differences were observed.

When the hIL12 and hIL7-carrying vaccinia virus-surrogate was administered as a single intratumoral injection to tumor bearing mice at 2×10⁷ pfu/mouse, the hIL12 and hIL7-carrying vaccinia virus-surrogate DNA was detected in tumor tissue and decreased time dependently. The hIL12 and hIL7-carrying vaccinia virus-surrogate DNA was BLQ in tumor tissue at 14 days after administration, except for 1 of 5 animals. The hIL12 and hIL7-carrying vaccinia virus-surrogate was BLQ in blood, brain, heart, kidney, lung, feces, ovary and urine. The hIL12 and hIL7-carrying vaccinia virus-surrogate DNA was detected in the following tissues: iliac lymph node, spleen, testis and uterus at 4 h after administration in 1 of 5 animals for each tissue; at 1 day after administration, the hIL12 and hIL7-carrying vaccinia virus-surrogate DNA was detected in liver tissue of 1 of 5 animals. The hIL12 and hIL7-carrying vaccinia virus-surrogate DNA was not detected in these tissues at later time points (1 day or 3 to 14 days after administration). No excretion of the hIL12 and hIL7-carrying vaccinia virus-surrogate DNA in urine or feces was detected. No remarkable sex differences were observed. With the exception of the tumor and iliac lymph node, human IL-7 and murine IL-12 were measured in tissues from those animals in which the hIL12 and hIL7-carrying vaccinia virus-surrogate DNA was detected. Human IL-7 and murine IL-12 were BLQ in the tissues examined.

When the hIL12 and hIL7-carrying vaccinia virus-surrogate was administered once intratumorally to male and female tumor-bearing BALB/c mice at 2×10⁷ pfu/mouse, The hIL12 and hIL7-carrying vaccinia virus-surrogate was detected in skin swabs at the injection site immediately after administration (within 2 minutes). The hIL12 and hIL7-carrying vaccinia virus-surrogate decreased time dependently and was BLQ at 3 days and later time points up to 21 days after administration. No remarkable sex differences were observed.

When the hIL12 and hIL7-carrying vaccinia virus was administered intravenously to cynomolgus monkeys at 3.4×10⁸ and 3.4×10⁹ pfu/kg once weekly for 4 weeks, the hIL12 and hIL7-carrying vaccinia virus DNA was detected in blood and decreased time dependently. At 3.4×10⁹ pfu/kg, the hIL12 and hIL7-carrying vaccinia virus DNA was detected in blood for 7 days after administration. The hIL12 and hIL7-carrying vaccinia virus DNA in blood increased with increasing dose. In tissues, the hIL12 and hIL7-carrying vaccinia virus DNA was detected only in spleen at 7 days after the fourth administration. The hIL12 and hIL7-carrying vaccinia virus DNA was detected in oral swab samples at 4 h and 1 day after administration and feces at 3 days after administration at 3.4×10⁹ pfu/kg. The IL12 and hIL7-carrying vaccinia virus DNA was not detected at later time points in oral swab samples or feces. The hIL12 and hIL7-carrying vaccinia virus DNA was BLQ in lacrimal swab samples or urine during the study. Overall, no remarkable sex differences were observed in biodistribution and shedding in cynomolgus monkeys.

### Example 20. Single Intravenous Dose Toxicity Study in Cynomolgus Monkeys Purpose

This non-GLP study was conducted to evaluate the potential toxicity of the hIL12 and hIL7-carrying vaccinia virus following a single intravenous injection in cynomolgus monkeys. In addition, the biodistribution of the hIL12 and hIL7-carrying vaccinia virus in tissue and blood was assessed and selected cytokine levels were measured.

### Study Design

A single intravenous dose of the hIL12 and hIL7-carrying vaccinia virus was administered to 2 male and 2 female cynomolgus monkeys per group at dose levels of 0 (vehicle: 30 mmol/L Tris-HCl, 10% sucrose, pH 7.6), 2.9×10⁷ or 2.9×10⁸ pfu/kg. Test article groups received a constant dosage volume of 5 mL/kg as a slow bolus injection over 5 minutes. One animal/sex/group was sacrificed 2 days after administration. The remaining animals were sacrificed 14 days after administration.

Mortality, morbidity and clinical signs were checked and recorded at least once daily until the scheduled sacrifice. Body weight and rectal temperature were recorded pretreatment and on day 1 (day of dosing) and days 2, 4, 8 and 15 posttreatment. Electrocardiogram (ECG), blood pressure and ophthalmology were evaluated once before treatment and on day 8 in surviving animals. Food consumption was checked daily.

Blood samples for the determination of cytokine and viral DNA levels in plasma were collected from all surviving animals during the pretreatment period and on days 1, 2, 3 (only for viral DNA levels), 4, 8 and 15.

Hematology, coagulation and blood biochemistry investigations were performed on all surviving animals pretreatment and on days 2, 4, 8 and 15 posttreatment. Urinalysis was performed pretreatment and on days 2, 8 and 15 posttreatment.

At the scheduled sacrifice, a full macroscopic postmortem examination was performed. Designated organs and tissues were weighed and preserved for microscopic examination and quantitative polymerase chain reaction investigations for biodistribution. A microscopic examination was performed on selected tissues from all animals.

### Results

No premature deaths occurred during the study and no toxicologically relevant clinical signs related to the treatment with the hIL12 and hIL7-carrying vaccinia virus were observed.

No effects on body weight or food consumption were noted at either dose level. Transient hyperthermia was noted in 1 animal in the high-dose group on day 2. No other treatment-related changes in rectal temperature were noted. There were no effects on cardiovascular parameters (ECG and blood pressure) and there were no ophthalmological findings at any dose level.

The determination of cytokine levels did not confirm a relationship between the human IL-7 and IL-12 p70 serum levels and the transgenes expression. A dose-related increase in monkey interferon gamma (IFN-γ) concentration was noted on day 2. No changes in tumor necrosis factor-alpha (TNF-α) concentration were noted at any dose level.

Viral DNA was not detected in liver, brain, heart, kidney, lung, testes, ovary or uterus samples at any time point in the biodistribution phase using the polymerase chain reaction detection method.

Viral DNA was quantified in spleen samples at ≥2.9×10⁷ pfu/kg on day 3, but was below the limit of quantification (BLQ) at later time points. It was also transiently quantified in blood samples at 2.9×10⁷ pfu/kg on day 1 and in blood samples at 2.9×10⁸ pfu/kg on days 1, 2 and 3, with a rapid clearance as no blood sample was positive for the viral DNA from day 4.

In hematology, all high-dose animals showed a moderately increased white blood cell count (×1.6 to ×2.5) and neutrophil count (×3.0 to ×4.3) and slightly to markedly decreased eosinophil (complete disappearance to ×0.8) and lymphocyte (×0.3 to ×0.6) counts on day 2. This was followed on day 8 by mild lymphocytosis in the surviving male and female and an increase in platelet count in the male only. In this same male, a slight increase in fibrinogen concentration was also seen on days 2 and 4. Platelet count decreased on day 2 in the other high-dose male. No changes were noted on day 15. These hematological changes were indicative of an inflammatory state in this group. They were considered to be test article-related but nonadverse because of their reversibility and the absence of associated clinical signs.

No effects of the test article were noted in blood biochemistry or urinalysis.

In animals sacrificed on day 3 or 15, there were no organ weight differences, gross findings or microscopic findings that were related to test article administration.

Conclusion

Under the experimental conditions of this study, a single intravenous injection of the hIL12 and hIL7-carrying vaccinia virus up to 2.9×10⁸ pfu/kg was well tolerated in cynomolgus monkeys.

The viral DNA was quantified in blood samples on day 1 at 2.9×10⁷ pfu/kg and on days 1, 2 and 3 at 2.9×10⁸ pfu/kg, with a rapid clearance since no blood sample was positive for the viral DNA from day 4.

The viral DNA was not detected in liver, brain, heart, kidney, lung, testes, ovary and uterus samples whatever the time point. The viral DNA was quantified only in spleen samples at ≥2.9×10⁷ pfu/kg on day 3, but BLQ on day 15.

### Example 21. Four-Week Repeated Intravenous Dose Toxicity Study in Mice Purpose

The objective of this GLP study was to evaluate the toxicity of the hIL12 and hIL7-carrying vaccinia virus during weekly intravenous injections administered to mice for 4 weeks. On completion of the treatment period, designated animals were held for a 4-week nontreatment period in order to evaluate the reversibility of any findings.

### Study Design

The hIL12 and hIL7-carrying vaccinia virus was intravenously administered to 10 male and 10 female CD-1 mice per group at dose levels of 0 (vehicle: 30 mmol/L Tris-HCl, 10% sucrose, pH 7.6), 8.5×10⁷, 8.5×10⁸ and 8.5×10⁹ pfu/kg once weekly for 4 weeks. The high dose level was the maximum feasible dose (MFD) based on the test item concentration (1.7×10⁹ pfu/mL) and the highest volume injectable intravenously to a mouse (5 mL/kg, repeated dose). Six additional males and 6 additional females were both included in the control and high-dose groups to be kept for the 4-week nontreatment period. In addition, 6 satellite males and 6 satellite females were included in each group for possible viremia, immunogenicity and cytokine measurements only.

The animals were checked twice daily for mortality. Clinical signs were recorded once daily. Body weight and food consumption were recorded at least once during the pretreatment period, on the day of treatment and at least once weekly through the end of the study. Body weight was also recorded each day for 3 days after the first and fourth administrations. Ophthalmological examinations were performed during the pretreatment period and at the end of the treatment period.

Blood samples for hematology and blood biochemistry investigations were collected at the end of the treatment and nontreatment periods. Blood samples were taken from satellite animals 2 days after the first administration for viremia determination and at the end of the treatment period for possible cytokine measurement and immunogenicity determination.

At the end of the treatment or nontreatment period, animals were euthanized and a full macroscopic postmortem examination was performed. Designated organs and tissues were weighed and preserved. A microscopic examination was performed on selected tissues.

Results

Weekly administration of the hIL12 and hIL7-carrying vaccinia virus for 4 weeks by the intravenous route did not result in any mortality. No treatment-related changes were observed in body weight, food consumption, ophthalmology or hematology in any dose level.

At doses ≥8.5×10⁷ pfu/kg, a slightly lower A/G ratio was observed, suggesting a higher globulin concentration compared to vehicle control. Increased spleen weight and cellularity of germinal centers in the spleen were also noted. These findings were considered to be associated with the test article.

At doses ≥8.5×10⁸ pfu/kg, enlarged spleens were noted (males and/or females). These findings were considered to be associated with the test article.

At a dose of 8.5×10⁹ pfu/kg, acute severe clinical signs after the third and fourth administration, such as hunched posture, piloerection, hypoactivity, bent head, decreased grasping reflex, loss of balance, dyspnea, half closed eyes, staggering gait and/or running in circles were noted. All of these clinical signs were observed within 15 to 30 minutes after administration and were generally not observed the day after. These clinical signs were suggestive of an immediate hypersensitivity reaction. They were, however, transient and had no effect on the overall condition of the animals. Enlarged iliac and inguinal lymph nodes (females), increased cellularity of germinal centers in the iliac, inguinal and mandibular lymph nodes (males and females) and increased incidence of minimal perivascular inflammation at the injection sites (males and females) were noted.

After the 4-week nontreatment period, the spleen and lymph nodes completely recovered in males and partially recovered in females. There was a complete recovery of the findings at the injection sites.

On day 3, viral DNA in the 8.5×10⁷ pfu/kg dose group was quantified in the blood of 3 of 6 females (geometric mean: 4.18×10² vg/μg of DNA) and no males. In the 8.5×10⁸ pfu/kg dose group, viral DNA was quantified in similar amounts in all animals but 1 male (2.29×10² vg/μg of DNA for males, 5.05×10² vg/μg of DNA for females). In the 8.5×10⁹ pfu/kg dose group, viral DNA was quantified in a higher amount in all animals (3.38×10³ vg/μg of DNA for males and 8.92×10³ vg/μg of DNA for females).

Conclusions

A dose level of 8.5×10⁹ pfu/kg resulted in adverse acute severe clinical signs after the third and fourth administration.

At dose levels of 8.5×10⁷ pfu/kg and 8.5×10⁸ pfu/kg, effects of the test article included a higher blood globulin concentration and a nonadverse increase in the cellularity of germinal centers in the spleen compared to vehicle control.

Consequently, under the experimental conditions of this study, the NOAEL (No Observed Adverse Effect Level) for the hIL12 and hIL7-carrying vaccinia virus was 8.5×10⁸ pfu/kg.

### Example 22. Four-Week Repeated Intravenous Dose Toxicity and Biodistribution Study in Cynomolgus Monkeys

### Purpose

This GLP study was conducted to evaluate the potential toxicity of the hIL12 and hIL7-carrying vaccinia virus during weekly intravenous injections administered to cynomolgus monkeys for 4 weeks. On completion of the treatment period, designated animals were held for a 4-week nontreatment period to evaluate the reversibility of any findings. In addition, biodistribution was assessed throughout the study period.

### Study Design

The hIL12 and hIL7-carrying vaccinia virus was intravenously administered to 3 male and 3 female cynomolgus monkeys per group at dose levels of 0 (vehicle: 30 mmol/L Tris-HCl, 10% sucrose, pH 7.6), 3.4×10⁷, 3.4×10⁸ and 3.4×10¹ pfu/kg once weekly for 4 weeks (administration on days 1, 8, 15 and 22). The animals at 3.4×10⁹ pfu/kg were assigned as satellite animals to evaluate biodistribution and shedding. The high dose level was the MFD based on the test item concentration (1.7×10⁹ pfu/mL) and the highest volume injectable intravenously to a cynomolgus monkey (2 mL/kg, repeated dose). The satellite animals at 3.4×10⁹ pfu/kg were terminated on day 15 due to findings noted after the second administration at this dose level. Therefore, 3 males and 3 females were additionally assigned as satellite animals to evaluate biodistribution and shedding in the 3.4×10⁸ pfu/kg group.

Two males and 2 females were added to the control group and 3.4×10⁸ pfu/kg group to assess the reversibility of toxicity findings observed during the dosing period.

For all animals, mortality, morbidity and clinical signs were checked and recorded at least twice daily during the study. Body weight was recorded pretreatment, on the day of treatment and at least once weekly through the end of the study. Food consumption was checked daily. Blood samples were collected for possible determination of antidrug antibodies.

For principal and recovery animals, rectal temperature was recorded once in the pretreatment period, 2 h after each administration, 1 day after each administration and 3 days after the first and fourth administration. ECG, blood pressure and ophthalmological examinations were performed pretreatment and at the end of the treatment period. Samples for hematology, coagulation and blood biochemistry investigations and samples for urinalysis were collected at the end of the treatment period. Blood samples were collected for possible determination of cytokine levels and viremia analysis at regular time points after the first and fourth administration. At the end of the treatment or nontreatment period, animals were sacrificed and a full macroscopic postmortem examination was performed. Designated organs and tissues were weighed and preserved. A microscopic examination was performed on selected tissues.

For group 3 satellite animals (at 3.4×10⁸ pfu/kg), oral and lacrimal swabs and blood, urine and feces samples were collected at regular time points throughout the study to determine biodistribution and shedding. For group 4 satellite animals (at 3.4×10⁹ pfu/kg), blood for hematology and biochemistry, and serum for additional investigations were collected on days 9 and 15 (before necropsy).

At the end of the treatment period, designated tissues were collected from group 3 satellite animals to evaluate biodistribution. On day 15, group 4 satellite animals were sacrificed and a full macroscopic postmortem examination was performed. Designated organs and tissues were weighed and preserved. A microscopic examination was performed on selected tissues.

### Results

No treatment-related changes were observed in food consumption, ECG, blood pressure, ophthalmology or urinalysis in any dose level.

At doses ≥3.4×10⁷ pfu/kg, an increase in spleen weight (males: ≥3.4×10⁷ pfu/kg, females: ≥3.4×10⁸ pfu/kg) and a nonadverse treatment-related increase in cellularity of germinal centers (males and females) in the spleen were noted. These changes were not noted at 3.4×10⁸ pfu/kg after the 4-week nontreatment period.

Mild to moderate decreases in mature red cell mass such as red blood cell count, hemoglobin concentration and hematocrit (males: ≥3.4×10⁸ pfu/kg, females: 3.4×10⁷ pfu/kg and 3.4×10⁸ pfu/kg) were noted. A mild decrease in mature red cell mass was noted in the control animals. In light of their amplitudes, these hematological changes were not considered to be adverse.

At doses of ≥3.4×10⁸ pfu/kg, enlarged spleens were noted (males and/or females). At a dose of 3.4×10⁸ pfu/kg, 1 male presented with hypoactivity on day 22, 4 h after the fourth administration that lasted <24 h. It was not considered to be adverse because of the low severity. In males, rectal temperature on day 2 increased when compared to pretreatment values (40.3° C. versus 39.5° C., respectively). Rectal temperatures returned to pretreatment temperatures on day 4. This change was not considered to be adverse, as it was transient and the magnitude of the change was minimal.

On day 8, after the second administration of 3.4×10⁹ pfu/kg in satellite animals, 1 male vomited and presented hypoactivity and prostration that evolved to ventral recumbency and a fixed stare. This animal was considered to be moribund and prematurely euthanized for humane reasons. In a histopathological examination, the cause of moribundity was considered to be a multi-organ systemic inflammatory reaction, mainly affecting the surface of thoracic and abdominal organs. Other animals presented hypoactivity 4 h after the second administration that lasted between 24 h and 48 h. One male presented prostration 4 h after the second administration. Body weight was decreased in the surviving animals. Considering the severe reactions following the second dose on day 8, treatment of the 5 other animals was discontinued and the animals were sacrificed on day 15.

Hematological changes consisted of mild to moderate decreases in mature red cell mass, alterations in platelet counts and/or mild to moderate increases in band neutrophils, lymphocyte, monocyte, large unstained cell and/or reticulocyte counts. Biochemical changes included mild to moderate decreases in sodium, chloride, phosphorus, albumin and total protein concentrations, mild to moderate increases in urea, creatinine and triglyceride concentrations, mild to moderate alterations in glucose concentration and mild to moderate increases in alkaline phosphatase, aspartate aminotransferase, alanine aminotransferase, creatinine kinase, lactate dehydrogenase and gamma-glutamyltransferase activities. These were indicative of increased erythrocyte turnover due to hemorrhage or decreased erythrocyte lifespan, an inflammatory and immunological reaction, impaired renal function, cholestasis and hepatobiliary and skeletal muscle cell injury. A multi-organ systemic inflammatory reaction together with a deteriorated general state were considered the most likely underlying causes for the described alterations.

In a histopathological examination, a small increase in inflammatory infiltrates was noted in various organs (heart, liver, lungs and body cavities/mesenteric fat) and was consistent with findings in the moribund animals. Bilateral testicular tubular degeneration was present in 1 male sacrificed on day 15. Secondary lesions in the epididymides were consistent with a toxic effect approximately 1 week earlier (i.e., at day 8 treatment). Similar but lower severity unilateral lesions were present in the testes of the 2 recovery animals at a dose of 3.4×10⁸ pfu/kg. The findings in the testes are unlikely to be a direct effect of the test article. However, the exact mechanism of the testicular findings could not be determined.

In the 3.4×10⁷ pfu/kg group, viral DNA was BLQ in any blood sample. In the 3.4×10⁸ pfu/kg group, viral DNA was quantified in 3 of 5 males (geometric mean: 4.26×10² vg/μg of DNA) and 4 of 5 females (3.78×10² vg/μg of DNA) on day 2 and in 2 of 5 males (2.34×10² vg/μg of DNA) and 1 of 5 females (1.42×10² vg/μg of DNA) on day 3. Viral DNA was BLQ on days 4 or 5. Viral DNA was quantified in 3 of 5 males (1.8×10² vg/μg of DNA) and 4 of 5 females (2.09×10³ vg/μg of DNA) on day 23 and in none of the 5 males and 1 of 5 females (4.03×10² vg/μg of DNA) on day 24. Viral DNA was not detected on day 25.

### Conclusions

Administration at 3.4×10⁷ pfu/kg and 3.4×10⁸ pfu/kg resulted in nonadverse findings with complete recovery as noted during in-life or histopathological examinations. Slight unilateral testicular degeneration was present in 2 animals at the recovery sacrifice. This was considered non-adverse because of the low severity of the lesions. At the high dose of 3.4×10⁹ pfu/kg, there were adverse treatment-related findings. Specifically, one male was euthanized because of severe deterioration in clinical condition, considered to be due to a multi-organ systemic inflammatory reaction, mainly affecting the surface of thoracic and abdominal organs. In addition, bilateral testicular degeneration was present in 1 male. This was not considered a direct effect of treatment, but was likely to be secondary to inflammatory changes resulting in pyrexia and interference with thermoregulation in the testis.

Consequently, under the experimental conditions of this study, the NOAEL ((No Observed Adverse Effect Level) for the hIL12 and hIL7-carrying vaccinia virus was estimated to be 3.4×10⁸ pfu/kg.

### Example 23. Five-Day Repeated Intratumoral Dose Toxicity Study of the hIL12 and hIL7-Carrying Vaccinia Virus-Surrogate in Tumor-Bearing Mice

### Purpose

The toxicity study in tumor-bearing mice was conducted to evaluate the potential toxicity of the virus when administered as an intratumoral injection, the clinical route of administration.

### Study Design

The hIL12 and hIL7-carrying vaccinia virus is a recombinant vaccinia virus carrying transgenes, human IL-12 and human IL-7. In this study, the hIL12 and hIL7-carrying vaccinia virus-surrogate carrying mouse IL-12 and human IL-7 was used since human IL-12 is not cross-reactive in the mouse. CT26.WT tumor cells were subcutaneously injected into the right flank of BALB/c mice at 3×10⁵ cells/50 μL/mouse. After establishment of the tumor (mean tumor volume: 75 to 81 mm³), the hIL12 and hIL7-carrying vaccinia virus-surrogate was administered intratumorally to 10 male and 10 female mice per group at dose levels of 0 (vehicle control: 30 mmol/L Tris-HCl, 10% sucrose, pH 7.6), 2×10⁵, 2×10⁶ and 2×10⁷ pfu/mouse/day by alternate-day administrations for 5 days (on days 1, 3 and 5). A dosage volume of 30 μL/mouse was used for all groups. The animals were sacrificed on day 15. The study parameters included clinical observations, body weight, food consumption, tumor volume, organ weight, necropsy and histopathology. In addition, human IL-7, mouse IL-12, mouse TNF-α and mouse IFN-γ concentrations in serum were evaluated on day 6 (24 h after the third administration) in the satellite animals (3 animals/article/sex/group).

### Results

At a dose ≥2×10⁵ pfu/mouse, a decrease in tumor size at the injection site was observed in males and an increase in focal necrosis in the tumor at the injection site was observed in males (except for 2×10⁶ pfu/mouse).

At a dose ≥2×10⁶ pfu/mouse, an increase in lymphoid infiltration in the tumor was observed in males and females, as well as an increase in the severity of fibrosis in the dermis and severity of macrophage infiltration in the tumor at the injection site. Lymphoid hyperplasia in the spleen was observed in males. A decrease in tumor size was observed in females.

At a dose of 2×10⁷ pfu/mouse, an increase in neutrophil infiltration in the tumor at the injection site was observed in males, as well as a decreased severity and incidence of extramedullary hematopoiesis in the spleen and decreased spleen weight. Lymphoid hyperplasia in the spleen was observed in females.

For cytokines, serum levels of mouse IL-12 and human IL-7 did not increase in either sex of any dose group with the exception of 1 male at a dose of 2×10⁶ pfu/mouse, where an increased concentration of mouse IL-12 was observed. The concentration of mouse IFN-γ was increased in males and females at 2×10⁶ and 2×10⁷ pfu/mouse. The concentration of mouse TNF-α was increased in males and females in all dose groups.

### Conclusions

The NOAEL (No Observed Adverse Effect Level) in the present study was estimated to be 2×10⁷ pfu/mouse for both sexes, since the histopathological changes were considered to be indicative of an activated immune system by the hIL12 and hIL7-carrying vaccinia virus-surrogate or the result of secondary changes associated with decreased tumor size, and thus, were not considered to be adverse.

### Summary of Examples 20-23

Major changes in mice treated with the hIL12 and hIL7-carrying vaccinia virus intravenously for 4 weeks were observed in the spleen (an increased cellularity of the germinal centers, characterized by an enlargement of germinal centers of splenic lymphoid follicles due to an increased number of lymphocytes) and the lymph nodes (iliac, inguinal and mandibular). The morphological changes in the lymph nodes appeared similar to that of the spleen. In cynomolgus monkeys, major organ changes following the 4-week intravenous dose were noted in the spleen (an increased cellularity of the germinal centers). These changes were indicative of an activated immune system and immune response to the hIL12 and hIL7-carrying vaccinia virus and considered to be nonadverse since these changes were in line with the pharmacological effect of the hIL12 and hIL7-carrying vaccinia virus and were reversible.

However, at the Maximum Feasible Dose (MFD), severe clinical signs probably related to the activated immune system or immune response were noted in mice and cynomolgus monkeys after repeated intravenous dosing. In mice, acute symptoms such as hunched posture, piloerection, hypoactivity, bent head, staggering gait, decreased grasping reflex, loss of balance and dyspnea were noted in mice on days 15 and 22, after the third and fourth dose. All of these clinical signs were observed within 15 to 30 minutes after administration and were generally not observed the day after. These transient clinical signs had no effect on the overall condition of the animals and were suggestive of an immediate hypersensitivity reaction. In cynomolgus monkeys, 1 male at the highest dose vomited and presented hypoactivity and prostration that evolved to ventral recumbency and a fixed stare on day 8, after the second administration. This animal was considered to be moribund and prematurely euthanized for humane reasons. Other animals presented hypoactivity 4 h after the second administration that lasted between 24 and 48 h. One male presented prostration 4 h after the second administration. In histopathological examination, the cause of moribundity was considered to be a multi-organ systemic inflammatory reaction, mainly affecting the surface of thoracic and abdominal organs.

These changes were noted only at the high dose, which was set as the MFD in each study based on the concertation of the drug substance and MFD volumes for animals from a humane perspective. In tumor-bearing mice, although similar histopathological changes indicative of an activated immune system by the hIL12 and hIL7-carrying vaccinia virus-surrogate were noted, no adverse findings were noted in any measurement.

In cynomolgus monkeys, 1 male at the highest dose showed a bilateral seminiferous tubule degeneration. The testicular finding was considered to represent an indirect effect of the hIL12 and hIL7-carrying vaccinia virus exposure. However, the exact pathogenesis of the testicular findings could not be determined.

Treatment-related toxicity findings noted in the repeat-dose toxicity studies in mice and cynomolgus monkeys and NOAELs are compiled in (Table 11).

TABLE 11
Treatment-related Toxicity Findings in 4-week Intravenous
Dose Toxicity Studies in Mice and Cynomolgus Monkeys

| | | NOAEL | |
|---|---|---|---|
| | | (pfu/kg) | |
| | | | Cynomolgus |
| System | Treatment-related Change | Mouse | Monkey |

Clinical Signs and General Condition

| | | | |
|---|---|---|---|
| Activated | Acute symptoms noted within | 8.5 × 10⁸ | NA |
| immune | 15 to 30 minutes after dosing | | |
| system/ | on days 15 and 22, such as | | |
| response | hunched posture, piloerection, | | |
| | hypoactivity, bent head, | | |
| | staggering gait, decreased | | |
| | grasping reflex, loss of | | |
| | balance and dyspnea | | |
| | Hypoactivity lasting more | NA | 3.4 × 10⁸ |
| | than 24 h after dosing on | | |
| | day 8, including prostration, | | |
| | ventral recumbency and | | |
| | moribundity | | |

Histopathology

| | | | |
|---|---|---|---|
| Reproductive | Testicular degeneration† | NA | 3.4 × 10⁸ |
| System | | | |

NA: not applicable; NOAEL: no-observed-adverse-effect level.
†The testicular finding was considered to represent an indirect effect of the hIL12 and hIL7-carrying vaccinia virus exposure.

### Example 24. A First-In-Human (FIH) Phase I Open-Label Dose Escalation and Dose Expansion Study of the hIL12 and hIL7-Carrying Vaccinia Virus

A Phase I open-label dose escalation and dose expansion study of the hIL12 and hIL7-carrying vaccinia virus is conducted in the United States.

### Overview

The study includes patients with advanced or metastatic solid tumors that are ineligible for surgical or medical treatment with curative intent and have progressed on or are ineligible for available standard therapy:
Group A: Cutaneous or subcutaneous tumors accessible for intratumoral injection.
Group B: Visceral lesions accessible for intratumoral injection with ultrasound or computed tomography (CT) guidance. Consideration may be given to endoscopically accessible lesions.

To be eligible for enrollment, patients have an Eastern Cooperative Oncology Group (ECOG) performance status 0 or 1 and measurable disease.

The study design includes a dose escalation phase and an RP2D expansion phase (FIG. 17). Planned enrollment is approximately 105 patients (21 to 30 in the dose escalation phase and approximately 75 in the dose expansion phase). Initially in the dose expansion phase, 60 patients are enrolled into the expansion cohorts. Based on responses observed in an expansion cohort, up to 15 additional patients with a specific tumor type may be added to further characterize the antitumor activity in that tumor type. More than 1 cohort may be expanded to include additional patients. The total number of patients in the expansion cohorts will depend on observed antitumor activity and biomarker immune response.

In the dose escalation phase, the proposed the hIL12 and hIL7-carrying vaccinia virus dose levels are 1×10⁷ pfu/mL, 1×10⁸ pfu/mL and 5×10⁸ pfu/mL. Each patient receives the assigned dose of the hIL12 and hIL7-carrying vaccinia virus monotherapy via intratumoral injection into the same tumor(s) on days 1 and 15 of the first 2 cycles (28-day cycles). At least 7 days must elapse between treatment of the first patient at each dose level and any subsequent patients at that level.

Patients are evaluated for dose-limiting toxicities (DLTs) during the first 28 days (Cycle 1). Safety and tolerability will be continually assessed from day 1 through 16 weeks after the last dose of the hIL12 and hIL7-carrying vaccinia virus, consistent with FDA feedback (FIG. 18)

For each dose level, after the planned number of evaluable patients (at least 3 patients) have completed the DLT observation period, safety for that dose level is assessed. Dose-escalation or de-escalation will be guided according to Bayesian Optimal Interval (BOIN) Design ([Liu & Yuan, 2015]), which is based on DLT occurrence.

A minimum of 4 weeks will elapse between completion of the DLT observation period for a given dose level and the first administration at the next dose level, to allow additional observation time for potential delayed reactions before initiating the next dose concentration level.

Enrollment and DLT evaluation of all cohorts in Group A will be completed prior to initiating enrollment in Group B. Group B dose escalation will begin at 1 dose level lower than the RP2D identified in Group A.

The primary objectives are to assess the safety and tolerability of the hIL12 and hIL7-carrying vaccinia virus and to determine the MTD and/or RP2D of the hIL12 and hIL7-carrying vaccinia virus for patients with advanced or metastatic cancer. The secondary objectives are to assess antitumor activity (based on percent change in size of tumors), objective response rate (ORR) of injected tumors, pharmacokinetics and viral shedding. Exploratory endpoints will evaluate additional measures of antitumor activity, including percent change from baseline in the sum of diameters of noninjected tumors, ORR of noninjected tumors, progression-free survival (PFS), time to progression (TTP), duration of response (DOR) and overall survival (OS), as well as pharmacodynamic and predictive biomarkers.

Viral shedding with follow-up viral infectivity assessments of positive samples are monitored.

### Dosing Rationale

The starting dose of the hIL12 and hIL7-carrying vaccinia virus for the FIH study is anticipated to be safe and minimally pharmacologically active, as supported by nonclinical studies. The hIL12 and hIL7-carrying vaccinia virus will be administered at a fixed concentration (pfu/mL), and the volume of dose will be adjusted based on tumor size. The starting dose concentration of the hIL12 and hIL7-carrying vaccinia virus is set to 1×10⁷ pfu/mL with up to 6 mL injected per single lesion and/or per dose per patient by intratumoral administration. The volume of injected hIL12 and hIL7-carrying vaccinia virus will depend on tumor size to ensure consistent virus exposure to tumor cells, which is estimated by injection ratio (virus volume injected/target tumor size).

Nonclinical pharmacology data discussed above in Examples 1-15, demonstrated that the minimum biologically active dose of the hIL12 and hIL7-carrying vaccinia virus in animal tumor models is 2×10⁵ pfu when the hIL12 and hIL7-carrying vaccinia virus-surrogate was administered intratumorally in a 30 μL volume to a 50 mm³ tumor (injection ratio of 0.6). Therefore, the minimum biologically active concentration inside a tumor (i.e., target injection site) is approximately 4×10⁶ pfu/cm³ tumor (=2×10⁵ pfu/50 mm³). Similar injection ratio is expected to be effective in human tumors; therefore, the hIL12 and hIL7-carrying vaccinia virus dose concentration in the clinical study will be a target similar to the the hIL12 and hIL7-carrying vaccinia virus-surrogate concentration (6.7×10⁶ pfu/mL=2×10⁵ pfu/30 μL) to achieve a minimum biologically active concentration in the tumor. Consequently, the initial dose concentration of this FIH study is estimated to be 1×10⁷ pfu/mL, with the volume of the hIL12 and hIL7-carrying vaccinia virus dose to inject into the tumor differing according to tumor size (categorized by longest dimension) to achieve the target range of an injection ratio of approximately 0.2 to 0.8.

The starting dose was also assessed according to the results of repeat dose nonclinical toxicology studies. The no-observed-adverse-effects level (NOAEL) after 4 weeks of intravenous dosing (once weekly; total of 4 doses) was estimated to be 8.5×10⁸ pfu/kg in mice and 3.4×10⁸ pfu/kg in monkeys. In addition, the NOAEL after intratumoral injection of the hIL12 and hIL7-carrying vaccinia virus-surrogate to mice was estimated to be 2×10⁷ pfu per tumor (maximum feasible dose [MFD]). The hIL12 and hIL7-carrying vaccinia virus is an oncolytic vaccinia virus engineered to replicate selectively in tumor cells, and nonclinical biodistribution study results support that the hIL12 and hIL7-carrying vaccinia virus selectively replicates in tumor cells after intratumoral administration. The impact on safety was conservatively estimated with whole-body-based exposure by utilizing toxicology study results of intravenous administration. The starting dose (Dose Level 1 in proposed FIH study) of 1×10⁷ pfu/mL is estimated to be approximately 1.0×10⁶ pfu/g (1×10⁷ pfu/mL administered in a volume up to 6 mL per 60-kg human). Therefore, the safety margin is more than 340-fold (3.4×10⁸/1.0×10⁶) compared to the NOAEL in the most sensitive species (cynomolgus monkey). The highest planned dose (Dose Level 3) is 5×10⁸ pfu/mL (MFD), which is approximately 5.0×10⁷ pfu/kg (5.0×10⁷ pfu/mL administered in a volume up to 6 mL per 60-kg human). Therefore, the highest planned dose is 6.8-fold (3.4×10⁸/5.0×10⁷) less than the NOAEL in the cynomolgus monkey. An intermediate dose level (Dose Level 2) of 1×10⁸ pfu/mL with a 10-fold increment from starting dose is planned.

The hIL12 and hIL7-carrying vaccinia virus will be given every 2 weeks in two 28-day cycles via intratumoral injection in the FIH study, as superior antitumor effect was demonstrated via repeat doses with a 2 week interval compared to single dose in nonclinical pharmacology study. Patients who have not met any individual treatment discontinuation criteria and are receiving clinical benefit may continue to the extended treatment period (continued 28-day cycles) as decided by the investigator.

### Viral Shedding

In this study, urine and saliva will be collected from all patients to monitor viral shedding. In addition, shedding analysis of skin will be performed for patients with cutaneous or subcutaneous accessible tumors (Group A).

Viral shedding will be monitored via detection of viral DNA by a validated quantitative polymerase chain reaction (qPCR) method with follow-up viral infectivity assessment of positive samples. In cycles 1 and 2, urine, saliva and skin (Group A only) samples will be collected predose, with dense monitoring performed 3 h, 6 h and 24 h after dosing, anytime on days 4 and 8 postdose. Sparse sampling will be performed after the last dosing cycle (at end of treatment [EOT]) and 2, 6 and 10 weeks after EOT as part of follow-up monitoring to assure complete elimination of the virus.

### Features of the Patient Population

Patients with advanced or metastatic solid tumors that are ineligible for surgical or medical treatment with curative intent and have progressed on or are ineligible for available standard therapy are enrolled. Patients must have measurable disease (Response Evaluation Criteria in Solid Tumors [RECIST]) and an ECOG performance status of 0 or 1. Patients with active or prior autoimmune or inflammatory disorders requiring systemic therapy within the past 2 years, including inflammatory skin conditions or severe eczema, inflammatory bowel disease, diverticulitis (with the exception of diverticulosis), celiac disease, systemic lupus erythematosus, sarcoidosis syndrome, Wegener syndrome, Graves' disease, rheumatoid arthritis, hypophysitis, uveitis, etc., will be excluded.

Patients with a known history of human immunodeficiency virus, hepatitis B surface antigen, hepatitis B core immunoglobulin M or immunoglobulin G antibody or hepatitis C indicating acute or chronic infection are excluded. Alterations in the immune systems of these patients may impact the characterization of the effects of study treatment on immune cell populations. The sponsor will assess whether to remove this exclusion criterion based on emerging data in this study.

The escalation cohorts will include patients with cutaneous or subcutaneous tumors accessible for intratumoral injection (Group A) and patients with visceral lesions accessible for intratumoral injection with ultrasound or CT guidance (Group B). Consideration may be given to endoscopically accessible lesions. The Group A (cutaneous/subcutaneous) expansion cohort will include the following tumor-specific cohorts: squamous cell carcinomas of the head and neck, dermatological, genitourinary/gynecological, gastrointestinal and other cutaneous/subcutaneously accessible solid tumors.

### Study Design

This Phase I Study will assess the safety, tolerability and pharmacokinetic profile and viral shedding of the hIL12 and hIL7-carrying vaccinia virus and will determine the MTD and/or RP2D in patients with advanced or metastatic solid tumors. In addition, the study will evaluate antitumor activity by the percent change in size of injected/noninjected tumors, ORR of injected/noninjected tumors, PFS, TTP, DOR and OS. Disease response and progression will be evaluated by the investigator according to RECIST 1.1 and immune-modified RECIST (imRECIST) criteria [Hodi et al, 2018]. imRECIST is an adaptation of immune-related RECIST and accounts for potential delayed responses that may be preceded by initial apparent radiographic progression, including appearance of new lesions.

In this study, the hIL12 and hIL7-carrying vaccinia virus will be administered as monotherapy; however, additional cohorts may be added by protocol amendment to further evaluate the hIL12 and hIL7-carrying vaccinia virus as a single agent and/or in combination with another anticancer agent (e.g., PD-1/PD-L1 inhibitor). The starting concentration of the hIL12 and hIL7-carrying vaccinia virus in the escalation phase is 1×10⁷ pfu/mL. The volume of the hIL12 and hIL7-carrying vaccinia virus to be injected per tumor is calculated according to the size of each target tumor to ensure consistent drug exposure within individual lesions. Lesions will be selected for injection by the investigator. The largest and/or most symptomatic lesions within the protocol-specified size range, should be prioritized for selection for injection with the hIL12 and hIL7-carrying vaccinia virus. Lesion selection may not change during cycles 1 and 2. The same tumors will be injected at each time point in cycles 1 and 2. Patients will have baseline and on-treatment biopsies on or before day 1 of cycles 1 and 2, respectively.

### Statistical Considerations

This study will enroll approximately 10⁵ patients. In the dose escalation phase, approximately 21 to 30 patients will be enrolled. The sample size is not based on a statistical power calculation. The number of patients enrolled will depend on the incidence of DLTs. The estimated number of patients should provide adequate information for the dose escalation and safety objectives of the study.

In the dose expansion phase, initially 60 patients will be enrolled into 6 tumor-specific expansion cohorts (10 patients per cohort). With the assumption that the true ORR in the injected tumors is 20%, the predictive probability of observing at least 1 responder in 10 patients would be approximately 89%. The total number of patients in the expansion cohorts will depend on observed antitumor activity and biomarker immune response.

An expansion cohort may increase in size to 25 patients to better assess the ORR across all tumors (i.e., not limited to injected tumors). With the assumption that the true ORR is at least 20%, the predictive probability of observing at least 5 responders in 25 patients would be 58%. For frequentist estimation of a proportion in a sample of 25 patients, a 90% 2-sided confidence interval for an observed response rate of 20% would have limits of (7%, 33%).

### Example 25. A Phase I Open-Label Monotherapy Study of the hIL12 and hIL7-Carrying Vaccinia Virus

A phase 1 open-label monotherapy study of the hIL12 and hIL7-carrying vaccinia virus in Japanese patients with advanced or metastatic solid tumors that are ineligible for surgical or medical treatment with curative intent and have progressed on or are ineligible for available standard therapy is conducted.

The study includes patients with visceral lesions accessible by intratumoral injection with ultrasound or CT guidance:

Group V1: Primary or metastatic tumors in the liver

Group V2: Primary or metastatic gastric tumors

The study includes a dose escalation phase and a dose expansion phase. The planned enrollment is up to 18 patients (Group V1) in the dose escalation phase and approximately 30 patients (20 in Group V1 and 10 in Group V2) in the dose expansion phase. An additional 10 patients (Group V3) may be added in the dose expansion phase to evaluate an additional tumor type yet to be determined.

For all patients, the study will consist of the following periods: screening (up to 28 days), initial treatment period (two 28-day cycles), optional extended treatment period (continued 28-day cycles) and follow-up period (safety and survival follow-up).

Patients will receive the assigned dose of the hIL12 and hIL7-carrying vaccinia virus monotherapy via intratumoral injection into the same tumor(s) on days 1 and 15 of each of the two 28-day cycles in the initial treatment period. Following cycle 2, patients who have not met any individual treatment discontinuation criteria and are receiving clinical benefit may continue to the extended treatment period as decided by the investigator. During the extended treatment period, patients will receive intratumoral administration of the hIL12 and hIL7-carrying vaccinia virus on days 1 and 15 of each cycle until treatment discontinuation criteria are met. In the extended treatment period, tumors previously not selected for intratumoral administration of the hIL12 and hIL7-carrying vaccinia virus may be treated (including those previously selected for biopsy).

The dose escalation phase will evaluate the safety and tolerability of the hIL12 and hIL7-carrying vaccinia virus and the MTD/RP2D in Japanese patients. Pending safety results from the dose escalation phase, dose expansion cohorts will open enrollment at least 4 weeks after the last patient in the dose escalation phase completes the DLT evaluation period.

Primary, secondary and exploratory objectives are similar to those of the FIH study in the United States described in Example 24.

### Example 26. A Phase I Open-Label Study of the hIL12 and hIL7-Carrying Vaccinia Virus

A phase 1 open-label study of the hIL12 and hIL7-carrying vaccinia virus (safety lead-in phase, followed by the hIL12 and hIL7-carrying vaccinia virus combination therapy with checkpoint inhibitors [CPIs]) is conducted in Chinese patients with advanced or metastatic solid tumors.

The study will include patients with advanced or metastatic solid tumors who are ineligible for curative treatment and have progressed on or are ineligible for available standard therapy:
Group A: Cutaneous or subcutaneous tumor(s) accessible by intratumoral injection, including patients with head and neck squamous cell carcinoma, nasopharyngeal cancer, sarcoma, genitourinary/gynecological cancer or other cutaneously/subcutaneously accessible solid tumors.
Group B: Liver metastases accessible by intratumoral injection with ultrasound or CT guidance (any primary tumor type).

The study includes a safety lead-in phase and an RP2D expansion phase. The planned enrollment is approximately 24 patients in the safety lead-in phase and 70 patients in the RP2D expansion phase.

In all parts of this study, the study periods will consist of screening (up to 28 days), treatment (two 28-day cycles), safety follow-up (16 weeks after the last dose) and survival follow-up (at least 12 weeks until death, withdrawal of consent or study closure).

All patients will be administered a total of 4 doses of the hIL12 and hIL7-carrying vaccinia virus by intratumoral injection (study days 1, 15, 29 and 43). In addition, the combination cohort of patients will receive CPI therapy via intravenous infusion starting on cycle 1, day 1 and continuing according to the local product label.

The primary objectives are to assess the safety and tolerability of the hIL12 and hIL7-carrying vaccinia virus as monotherapy and in combination with CPI therapy and to determine the RP2D in Chinese patients. Secondary and exploratory objectives are similar to those of the FIH study in the United States described in Example 24.

1. A pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region; and
a pharmaceutically acceptable carrier.

2. The pharmaceutical composition of claim 1, wherein the pharmaceutically acceptable carrier comprises tromethamine and sucrose: optionally wherein the pharmaceutically acceptable carrier comprises tromethamine at a concentration of about 10 mmol/L to about 50 mmol/L; and/or wherein the pharmaceutically acceptable carrier comprises sucrose at a concentration of about 5% w/v to about 15% w/v.

3-4. (canceled)

5. The pharmaceutical composition of claim 1, wherein the pH of the composition is about 5.0 to about 8.5.

6. A pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region;
tromethamine at a concentration of about 10 mmol/L to about 50 mmol/L; and
sucrose at a concentration of about 5% w/v to about 15% w/v,
wherein the pH of the composition is about 5.0 to about 8.5.

7. The pharmaceutical composition of claim 1, wherein (a) the deletion in the SCR domains in the B5R membrane protein extracellular region comprises a deletion in SCR domains 1-4;
(b) the deletion in the SCR domains in the B5R membrane protein extracellular region comprises amino acid residues 22-237 of the amino acid sequence set forth in GenBank Accession No. AAA48316.1;
(c) the gene encoding the SCR domain-deleted B5R region is a gene encoding a polypeptide containing the signal peptide, stalk, transmembrane, and cytoplasmic tail domains of the B5R region; and/or
(d) the SCR domain-deleted B5R region comprises the amino acid sequence of the B5R region corresponding to the amino acid sequence set forth in SEQ ID NO: 2.

8-10. (canceled)

11. The pharmaceutical composition of claim 1, wherein the oncolytic vaccinia virus is a LC16mo strain of virus; optionally wherein the oncolytic vaccinia virus is LC16mO ΔSCR VGF-SP-IL12/O1L-SP-IL7.

12. (canceled)

13. The pharmaceutical composition of claim 1, comprising about 1×10⁷ to about 1×10⁹ particle forming units (pfu)/ml of the oncolytic vaccinia virus: optionally comprising about 1×10⁷, about 5×10⁷, about 1×10⁸, about 5×10⁸, about 1×10⁹, or about 5×10⁹ particle forming units (pfu)/ml of the oncolytic vaccinia virus.

14-19. (canceled)

20. The pharmaceutical composition of claim 2, wherein the concentration of tromethamine is about 15 mmol/L to about 45 mmol/L; 20 mmol/L to about 40 mmol/L; or 25 mmol/L to about 35 mmol/L: optionally wherein the concentration of tromethamine is about 30 mmol/L.

21. (canceled)

22. The pharmaceutical composition of claim 2, wherein the concentration of sucrose is about 6% w/v to about 14% w/v; about 7% w/v to about 13% w/v; about 8% w/v to about 12% w/v; or about 9% w/v to about 11% w/v; optionally wherein the concentration of sucrose is about 10% w/v.

23. (canceled)

24. The pharmaceutical composition of claim 5, wherein the pH of the composition is about 6.0 to about 8.0; about 6.5 to about 8.0; or about 6.8 to about 7.8: optionally wherein the pH of the composition is about 7.6.

25. (canceled)

26. The pharmaceutical composition of claim 1, wherein the composition is stable for at least about 6 months to about 2 years when stored at about −70° C.

27. A vial comprising the pharmaceutical composition of claim 1.

28. A syringe comprising the pharmaceutical composition of claim 1.

29. A method of treating a subject having a cancer, comprising administering to the subject a therapeutically effective amount of a pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region; and
a pharmaceutically acceptable carrier, thereby treating the subject.

30. A method of treating a subject having a cancer, comprising administering to the subject a therapeutically effective amount of a pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region; and
a pharmaceutically acceptable carrier,
wherein administration of the pharmaceutical composition to the subject induces an abscopal effect, thereby treating the subject.

31. A method of inducing an abscopal effect in a subject having a cancer, comprising administering to the subject a therapeutically effective amount of a pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region; and
a pharmaceutically acceptable carrier, thereby inducing an abscopal effect in a subject having a cancer.

32. A method of treating a subject having a cancer, comprising administering to the subject a therapeutically effective amount of a pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region;
tromethamine at a concentration of about 10 mmol/L to about 50 mmol/L; and
sucrose at a concentration of about 5% w/v to about 15% w/v,
wherein the pH of the composition is about 5.0 to about 8.5, thereby treating the subject.

33. A method of treating a subject having a cancer, comprising administering to the subject a therapeutically effective amount of a pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region;
tromethamine at a concentration of about 10 mmol/L to about 50 mmol/L; and
sucrose at a concentration of about 5% w/v to about 15% w/v,
wherein the pH of the composition is about 5.0 to about 8.5, and
wherein administration of the pharmaceutical composition to the subject induces an abscopal effect, thereby treating the subject.

34. A method of inducing an abscopal effect in a subject having a cancer, comprising administering to the subject a therapeutically effective amount of a pharmaceutical composition comprising,
about 1×10⁶ to about 1×10¹⁰ particle forming units (pfu)/ml of an oncolytic vaccinia virus, wherein the oncolytic vaccinia virus comprises in its genome a polynucleotide encoding human interleukin-7 and a polynucleotide encoding human interleukin-12, lacks a functional virus growth factor (VGF) protein and a functional O1L protein, and has a deletion in the SCR domains in the B5R membrane protein extracellular region;
tromethamine at a concentration of about 10 mmol/L to about 50 mmol/L; and
sucrose at a concentration of about 5% w/v to about 15% w/v,
wherein the pH of the composition is about 5.0 to about 8.5, and
wherein administration of the pharmaceutical composition to the subject induces an abscopal effect, thereby inducing an abscopal effect in the subject.

35. The method of claim 30, wherein the abscopal effect occurs in a metastatic tumor that is proximate to a primary solid tumor: or wherein the abscopal effect occurs in a metastatic tumor that is remote to a primary solid tumor.

36. (canceled)

37. The method of claim 29, wherein the oncolytic vaccinia virus is LC16mO ΔSCR VGF-SP-IL12/O1L-SP-IL7.

38. The method of claim 29, wherein the subject is administered a dose of about 1×10⁷ to about 1×10⁹ particle forming units (pfu); optionally the subject is administered a dose of about 1×10⁷, about 5×10⁷, about 1×10⁸, about 5×10⁸, about 1×10⁹, or about 5×10⁹ particle forming units (pfu).

39-43. (canceled)

44. The method of claim 29, wherein the administration is intratumoral administration.

45. The method of claim 29, wherein the dose of the pharmaceutical composition is administered to the subject intratumorally in a volume that achieves an injection ratio of about 0.2 to about 0.8 (volume of pharmaceutical composition/tumor volume).

46. The method of claim 29, wherein the pharmaceutical composition is administered to the subject once about once every week, once every two weeks, once every three weeks, or once every four weeks; or (b) is administered to the subject in a dosing regimen.

47-50. (canceled)

51. The method of claim 29, wherein the cancer is (a) a primary tumor;
(b) a metastatic tumor;
(c) a cutaneous, subcutaneous, mucosal or submucosal tumor;
(d) a primary or metastatic solid tumor in a location other than a cutaneous, a subcutaneous, a mucosal or a submucosal location;
(e) a head and neck squamous cell carcinoma, a dermatological cancer, a nasopharyngeal cancer, a sarcoma, or a genitourinary/gynecological tumor;
(f) a primary or metastatic tumor of the liver;
(g) a primary or metastatic gastric tumor; and/or
(h) malignant melanoma, lung adenocarcinoma, lung cancer, small cell lung cancer, lung squamous carcinoma, kidney cancer, bladder cancer, head and neck cancer, breast cancer, esophageal cancer, glioblastoma, neuroblastoma, myeloma, ovarian cancer, colorectal cancer, pancreatic cancer, prostate cancer, hepatocellular carcinoma, mesothelioma, cervical cancer or gastric cancer.

52-60. (canceled)

61. The method of claim 29, wherein the subject is human, optionally wherein the subject is an adult subject, an adolescent subject, or a pediatric subject.

62-64. (canceled)

65. The method of claim 29, wherein administration of the pharmaceutical composition to the subject leads to at least one effect selected from the group consisting of inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, antitumor immunity, and stable disease.

66. The method of claim 29, further comprising administering to the subject an additional therapeutic agent or therapy, wherein the additional therapeutic agent or therapy, is selected from the group consisting of surgery, radiation, a chemotherapeutic agent, a cancer vaccine, a checkpoint inhibitor, a lymphocyte activation gene 3 (LAG3) inhibitor, a glucocorticoid-induced tumor necrosis factor receptor (GITR) inhibitor, a T-cell immunoglobulin and mucin-domain containing-3 (TIM3) inhibitor, a B- and T-lymphocyte attenuator (BTLA) inhibitor, a T cell immunoreceptor with Ig and ITIM domains (TIGIT) inhibitor, a CD47 inhibitor, an indoleamine-2,3-dioxygenase (IDO) inhibitor, a bispecific anti-CD3/anti-CD20 antibody, a vascular endothelial growth factor (VEGF) antagonist, an angiopoietin-2 (Ang2) inhibitor, a transforming growth factor beta (TGFβ) inhibitor, a CD38 inhibitor, an epidermal growth factor receptor (EGFR) inhibitor, granulocyte-macrophage colony stimulating factor (GM-CSF), cyclophosphamide, an antibody to a tumor-specific antigen, Bacillus Calmette-Guerin vaccine, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement.

67-75. (canceled)

76. A pharmaceutical composition comprising,
about 1×10⁶ to about 1×10¹⁰ particle forming units (pfu)/ml of LC16mO ΔSCR VGF-SP-IL12/O1L-SP-IL7;
tromethamine at a concentration of about 30 mmol/L; and
sucrose at a concentration of about 10% w/v,
wherein the pH of the composition is about 7.6.

77. A method of treating a subject having a cancer, comprising administering to the subject a therapeutically effective amount of a pharmaceutical composition comprising,
about 1×10⁶ to about 1×10¹⁰ particle forming units (pfu)/ml of LC16mO ΔSCR VGF-SP-IL12/O1L-SP-IL7;
tromethamine at a concentration of about 30 mmol/L; and
sucrose at a concentration of about 10% w/v,
wherein the pH of the composition is about 7.6, thereby treating the subject.

78. A method of treating a subject having a cancer, comprising administering to the subject a therapeutically effective amount of a pharmaceutical composition comprising,
about 1×10⁶ to about 1×10¹⁰ particle forming units (pfu)/ml of LC16mO ΔSCR VGF-SP-IL12/O1L-SP-IL7;
tromethamine at a concentration of about 30 mmol/L; and
sucrose at a concentration of about 10% w/v,
wherein the pH of the composition is about 7.6, and
wherein administration of the pharmaceutical composition to the subject induces an abscopal effect, thereby treating the subject.

79. A method of inducing an abscopal effect in a subject having a cancer, comprising administering to the subject a therapeutically effective amount of a pharmaceutical composition comprising,
about 1×10⁶ to about 1×10¹⁰ particle forming units (pfu)/ml of LC16mO ΔSCR VGF-SP-IL12/O1L-SP-IL7;
tromethamine at a concentration of about 30 mmol/L; and
sucrose at a concentration of about 10% w/v,
wherein the pH of the composition is about 7.6, and
wherein administration of the pharmaceutical composition to the subject induces an abscopal effect, thereby inducing an abscopal effect in the subject.


SEQUENCE LISTING (2 sequences)

SEQ ID NO: 1 - protein, 317 aa, Vaccinia virus
MKTISVVTLLCVLPAVVYSTCTVPTMNNAKLTSTETSFNDKQKVTFTCDQGYHSLDPNAVCETDKWKYENPCKKMCTVSDYVSELYDKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPILPTCVRSNEKFDPVDDGPDDETDLSKLSKDVVQYEQEIESLEATYHIIIVALTIMGVIFLISVIVLVCSCDKNNDQYKFHKLLP

SEQ ID NO: 2 - protein, 101 aa, Artificial Sequence
  source: /note=“Description of Artificial Sequence Synthetic polypeptide”
MKTISVVTLLCVLPAVVYSTCVRSNEKFDPVDDGPDDETDLSKLSKDVVQYEQEIESLEATYHIIIVALTIMGVIFLISVIVLVCSCDKNNDQYKFHKLLP